(19) United States
(12) Patent Application Publication
(10) Pub. No.: US 2016/0345595 A1
(43) Pub. Date: Dec. 1, 2016

(54) FOODSTUFF

(71) Applicant: DUPONT NUTRITION BIOSCIENCES APS, Copenhagen K (DK)

(72) Inventors: Pablo Kuri-Brena Romero De Terreros, Mexico City (MX); Gerardo Avilez, Mexico City (MX); Charlotte Poulsen, Braband (DK); Rie Mejldal, Ostbirk (DK); Rikke Hoeegh Lorentsen, Randers (DK)

(21) Appl. No.: 15/114,925

(22) PCT Filed: Jan. 28, 2015
(86) PCT No.: PCT/EP2015/051713

§ 371 Date: Jul. 28, 2016

(30) Foreign Application Priority Data
Jan. 31, 2014 (GB) 1401676.0

(51) Int. Cl. A21D 8/04; A21D 13/00; C12N 9/24; A23L 7/104; A23L 7/117; A21D 2/26; A23L 29/00

(52) U.S. Cl. CPC A21D 8/042; A21D 2/267; A21D 13/0074; A23L 29/06; A23L 7/107; A23L 7/117; C12N 9/2482; C12Y 302/01008; A23V 2002/00

(57) ABSTRACT

A process for the preparation of a corn-based foodstuff, the process comprising the step of contacting a corn-based flour with a xylanase enzyme, as defined herein, such that a xylan-containing material native to the corn is degraded, is disclosed. Flours, masas and masa foodstuffs, especially tortillas, produced by the process are also disclosed.

### FIELD OF THE INVENTION

The present invention relates to a corn-based foodstuff, especially a masa foodstuff. In particular, the present invention relates to a masa foodstuff produced using a xylanase enzyme.

### BACKGROUND TO THE INVENTION

Corn provides the base ingredient for many staple foodstuffs. For example, corn may be processed to produce masa. Masa is the raw material for production of products such as corn tortilla, soft tortilla, corn chips, tortilla chips, taco shells, tamales, and corn flakes and corn bugles.

Other non-masa corn-based products, such as corn flakes and corn bugles are also known in the art. They are generally produced from corn-based flour by using an extrusion process.

Masa is typically produced by an alkaline cooking process, generally referred to as a nixtamalisation process. The nixtamalisation process involves cooking corn which still carries its outer shell (the pericarp). The cooking is performed in an alkaline solution such as lime (calcium hydroxide) and generally is for 12 to 24 hours. The cooked product is then steeped and washed to produce nixtamal. The nixtamal is then stone-ground to produce a flour (described in this specification as a “nixtamalised” corn flour”, which is mixed with water to produce a soft moist dough called masa.

After production masa may then be treated in a number of ways. The masa may be introduced into, for example, a tortilla mould or a tortilla sheeter. This is the traditional end use for the masa. In an alternative, the masa can be dried and milled into a “shelf-stable” flour product. The masa may be reconstituted from the flour product at a later stage and then formed into a food product, such as tortilla.

With regard to industrial implementation, typically masa is sold in the form of the dried masa or is formed into a final food product, such as a tortilla, which is then packed. In both of these aspects, one of the advantages of providing the product in this form is that the end user is free of the need to prepare the nixtamal and masa from the corn constituent. The requirements for labour, energy and processing time for the end use are reduced. Moreover, the product is simple to use.

For many years, endo-β-1,4-xylanases (EC 3.2.1.8) (referred to herein as xylanases) have been used for the modification of complex carbohydrates derived from plant cell wall material. It is well known in the art that the functionality of different xylanases (derived from different microorganisms or plants) differs enormously. Xylanase is the name given to a class of enzymes which degrade the linear polysaccharide beta-1,4-xylan into xylooligosaccharides or xylose, thus breaking down hemicellulose, one of the major components of plant cell walls.

The use of enzymes, including xylanases, as additives to corn is discussed generally in the prior art. For example, L. C. Platt-Lucero et al, Journal of Food Process Engineering 36 (2013) 179-186, describes the effect of a Bacillus subtilis xylanase on extruded, nixtamalised corn flour, the preparation of corn tortillas and the effect of the xylanase on viscosity and the sensory evaluation of the product.

### SUMMARY OF THE INVENTION

It has been surprisingly found by the present inventors that the incorporation of specific xylanase enzymes (as defined below) into corn flour allows a masa foodstuff to be produced from the corn-based flour, the masa foodstuff exhibiting improved characteristics such as texture, resistance, foldability and viscosity, compared with masa products prepared without such enzymes.

According to one aspect of the invention, there is provided a process for the preparation of a corn-based foodstuff, the process comprising the step of contacting a corn-based flour with a xylanase enzyme, such that a xylan-containing material native to the corn is degraded;

wherein the xylanase enzyme is selected from the group consisting of
(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided a process for the preparation of a corn-based foodstuff, the process comprising the step of contacting a corn-based flour with a xylanase enzyme, wherein the xylanase enzyme is selected from the group consisting of

(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided a process for preparing a masa comprising forming a mixture of a corn-based flour and xylanase enzyme as described above, and adding water to the mixture of corn-based flour and xylanase enzyme to form a masa.

According to one aspect of the invention, there is provided a process for preparing a masa comprising forming a masa as described above and processing the masa into a masa foodstuff. In particular aspects, the masa foodstuff is a tortilla.

According to one aspect of the invention, there is provided a corn-based flour comprising a xylanase enzyme, wherein the xylanase enzyme is selected from the group consisting of

(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

In particular aspects, the flour at least partially comprises a nixtamalised corn flour.

In particular aspects, the flour further includes a hydrocolloid.

According to one aspect of the invention, there is provided a corn-based foodstuff (such as a masa foodstuff) obtainable or obtained by a process as defined above.

According to one aspect of the invention, there is provided a masa obtainable or obtained by a process comprising:

(a) preparing a corn-based flour as defined above; and
(b) adding water to the flour to form a masa.

According to one aspect of the invention, there is provided a masa foodstuff obtainable or obtained by a process of:

(a) preparing a masa as defined above; and
(b) processing the masa to form the masa foodstuff.

In particular aspects, the masa foodstuff is a tortilla.

According to one aspect of the invention, there is provided a process for the preparation of a masa foodstuff, the process comprising the steps of

(i) cooking corn in an alkaline solution;
(ii) contacting a xylanase enzyme with the corn during or after cooking, wherein the xylanase enzyme is selected from the group consisting of
(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided a process for the preparation of a masa foodstuff, the process comprising the steps of

(i) cooking corn in an alkaline solution;
(ii) contacting a xylanase enzyme with the corn during or after cooking, such that a xylan-containing material native to the corn is degraded;
wherein the xylanase enzyme is selected from the group consisting of
(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided a process for the preparation of a masa foodstuff, the process comprising the steps of

(i) cooking corn in an alkaline solution;
(ii) contacting a xylanase enzyme with the corn during or after cooking, such that a xylan-containing material native to the corn is degraded;
wherein the xylanase enzyme is selected from the group consisting of
(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided use of a xylanase enzyme to improve the texture of a masa foodstuff, wherein the xylanase enzyme is selected from the group consisting of

(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided use of a xylanase enzyme to improve the resistance of a masa foodstuff, wherein the xylanase enzyme is selected from the group consisting of

(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided use of a xylanase enzyme to improve the foldability of a masa foodstuff, wherein the xylanase enzyme is selected from the group consisting of

(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

According to one aspect of the invention, there is provided use of a xylanase enzyme to modify the viscosity of a masa foodstuff, wherein the xylanase enzyme is selected from the group consisting of

(B) a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3) or
(A2) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

### BRIEF DESCRIPTION OF THE FIGURES

FIG. 1 shows a polypeptide sequence (SEQ ID No. 1) of xylanase A1 used in the present invention (FveXyn4). This is the pre-pro-protein. Underlined (lower case) portion of the sequence reflects an N terminal signal peptide can be cleaved before the enzyme is matured. The amino acids shown in bold and italicized may also be cleaved by post-translational modification before the enzyme is fully matured.

FIG. 2 shows a polypeptide sequence (SEQ ID No. 2) of xylanase A1 used in the present invention (FveXyn4). This is the pro-protein. The amino acids shown in bold and italicized may also be cleaved by post-translational modification before the enzyme is fully matured.

FIG. 3 shows a polypeptide sequence (SEQ ID No. 3) of xylanase A1 used in the present invention (FveXyn4). This is the active form of the enzyme. This may be referred to herein as the mature form of the enzyme.

FIG. 4 shows a nucleotide sequence (SEQ ID No. 4) encoding xylanase A1 used in the present invention (FveXyn4). The lower case nucleotides which are in bold show the intron sequence. The signal sequence is shown bold (upper case).

FIG. 5 shows a nucleotide sequence (SEQ ID No. 5) encoding xylanase A1 used in the present invention (FveXyn4). The signal sequence is shown bold (upper case).

FIG. 6 shows a nucleotide sequence (SEQ ID No. 6) encoding xylanase A1 used in the present invention (FveXyn4).

FIG. 7 shows a plasmid map of pZZH254.

FIG. 8 shows a polypeptide sequence (SEQ ID No. 7) of xylanase A2 used in the present invention (FoxXyn2). This is the pre-pro-protein. Underlined (lower case) portion of the sequence may reflect an N terminal signal peptide which can be cleaved before the enzyme is matured. The amino acids shown in bold and italicized may also be cleaved by post-translational modification before the enzyme is fully matured.

FIG. 9 shows a polypeptide sequence (SEQ ID No. 8) of xylanase A2 used in present invention (FoxXyn2). This is the pro-protein. The amino acids shown in bold and italicized may also be cleaved by post-translational modification before the enzyme is fully matured. This sequence may be an active form of the protein and may be one active form of the protein. This may be referred to herein as the mature form of the enzyme.

FIG. 10 shows a polypeptide sequence (SEQ ID No. 9) of xylanase A2 used in present invention (FoxXyn2). This is another active form of the enzyme. In some embodiments, this may be referred to herein as the mature form of the enzyme.

FIG. 11 shows a nucleotide sequence (SEQ ID No. 11) encoding xylanase A2 used in the present invention (FoxXyn2). The lower case nucleotides which are in bold show the intron sequence. The signal sequence is shown bold (upper case).

FIG. 12 shows a nucleotide sequence (SEQ ID No. 12) encoding xylanase A2 used in the present invention (FoxXyn2). The signal sequence is shown bold (upper case).

FIG. 13 shows a nucleotide sequence (SEQ ID No. 13) encoding xylanase A2 used in the present invention (FoxXyn2).

FIG. 14 shows a plasmid map of pZZH135.

FIG. 15 shows a nucleotide sequence (SEQ ID No. 14) encoding a xylanase for use in the present invention from Fusarium—obtained from Fusarium Comparative Sequencing Project, Broad Institute of Harvard and MIT (http://www.broadinstitute.org/)). The lower case nucleotides which are in bold show the intron sequence. The signal sequence is shown bold (upper case). Changes compared with SEQ ID No. 4 are underlined.

FIG. 16 shows a nucleotide sequence (SEQ ID No. 15) encoding a xylanase for use in the present invention from Fusarium—obtained from Fusarium Comparative Sequencing Project, Broad Institute of Harvard and MIT (http://www.broadinstitute.org/)). The signal sequence is shown bold (upper case). Changes compared with SEQ ID No. 5 are underlined.

FIG. 17 shows a nucleotide sequence (SEQ ID No. 16) encoding a xylanase for use in the present invention from Fusarium—obtained from Fusarium Comparative Sequencing Project, Broad Institute of Harvard and MIT (http://www.broadinstitute.org/). Changes compared with SEQ ID No. 6 are underlined.

FIG. 18 shows a nucleotide sequence (SEQ ID No. 20) encoding the xylanase B (AclXyn5) used in the present invention. The nucleotides which are in lowercase show the intron sequence. The signal sequence is shown bold (upper case).

FIG. 19 shows a nucleotide sequence (SEQ ID No. 21) encoding the xylanase B (AclXyn5) used in the present invention. The signal sequence is shown bold (upper case).

FIG. 20 shows a nucleotide sequence (SEQ ID No. 22) encoding the xylanase B (AclXyn5) used in the present invention.

FIG. 21 shows a polypeptide sequence (SEQ ID No. 17) of the xylanase B (AclXyn5) used in the present invention. This is the pre-protein. The bolded portion of the sequence reflects an N terminal signal peptide which can be cleaved before the enzyme is matured.

FIG. 22 shows a polypeptide sequence (SEQ ID No. 18) of the xylanase B (AclXyn5) used in the present invention. This is an active form of the enzyme. This may be referred to herein as the mature form of the enzyme.

FIG. 23 shows a polypeptide sequence (SEQ ID No. 19) of the xylanase B (AclXyn5) used in the present invention. This is also an active form of the enzyme which may arise from posttranslational processing.

FIG. 24 is a plasmid map of pZZH 159.

FIG. 25 illustrates the effect of xylanase A1 (as defined herein) and xylanase B (as defined herein, alone and in combination with carboxymethyl cellulose) on the viscosity of an alkaline corn masa;

FIG. 26 illustrates the effect of xylanase B in combination with carboxymethyl cellulose on the viscosity of an alkaline corn masa;

FIG. 27 illustrates tortillas produced from an alkaline masa after 10 days shelf life, showing the effect of xylanase B in combination with 0.5% of GRINDSTED CMC MAS 550 in comparison with control and other commercial enzymes after alkaline cooking;

FIG. 28 illustrates tortillas produced from an acidic masa after 10 days shelf life, showing the effect of xylanase A1 in comparison with control and other commercial enzymes after alkaline cooking;

FIG. 29 shows nucleotide sequences (without introns) of the coding sequences of variant GH10 xylanases in accordance with the present invention (SEQ ID No. 29, SEQ ID No. 30, SEQ ID No. 31, SEQ ID No. 32, and SEQ ID No. 33).

FIG. 30 shows nucleotide sequences (with introns shown underlined) of the coding sequences of variant GH10 xylanases in accordance with the present invention (SEQ ID No. 34, SEQ ID No. 35, SEQ ID No. 36, SEQ ID No. 37, and SEQ ID No. 38).

FIG. 31 shows amino acid sequences of mature variant GH10 xylanases in accordance with the present invention (SEQ ID No. 39, SEQ ID No. 40, SEQ ID No. 41, SEQ ID No. 42, and SEQ ID No. 43).

FIG. 32 shows a plasmid map of pEntry-FveXyn4.

FIG. 33 shows plasmid maps of pTTT-pyr2 (SpeKpn), pTTT-pyr2-FveXyn4 and pTTT-pyr2-FveXyn4 variant.

FIG. 34 shows a polypeptide sequence (SEQ ID No. 44) of a xylanase from Fusarium—Fusarium Comparative Sequencing Project, Broad Institute of Harvard and MIT (http://www.broadinstitute.org/)). In some embodiments, this sequence is a backbone sequence.

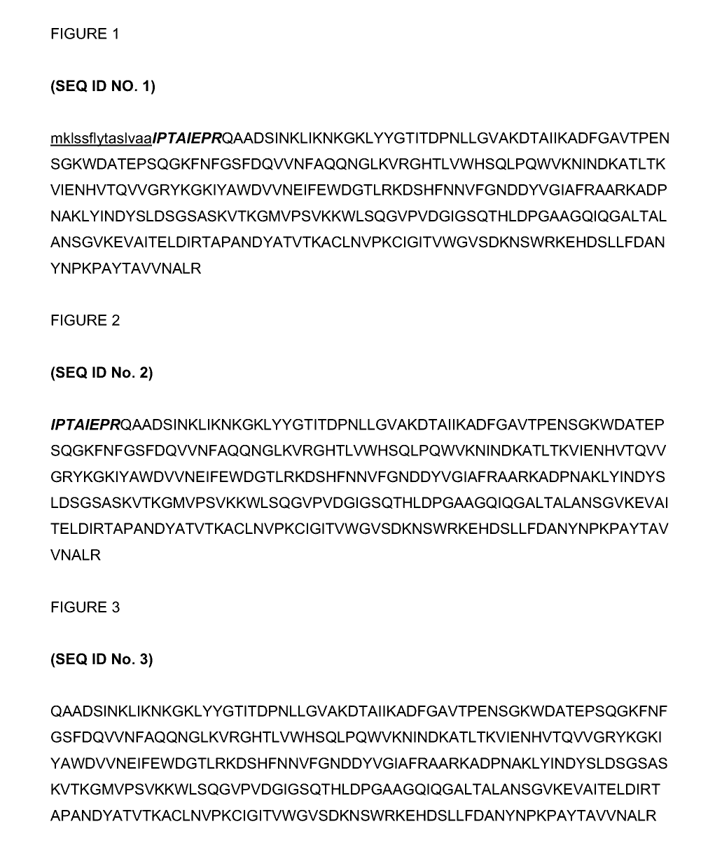

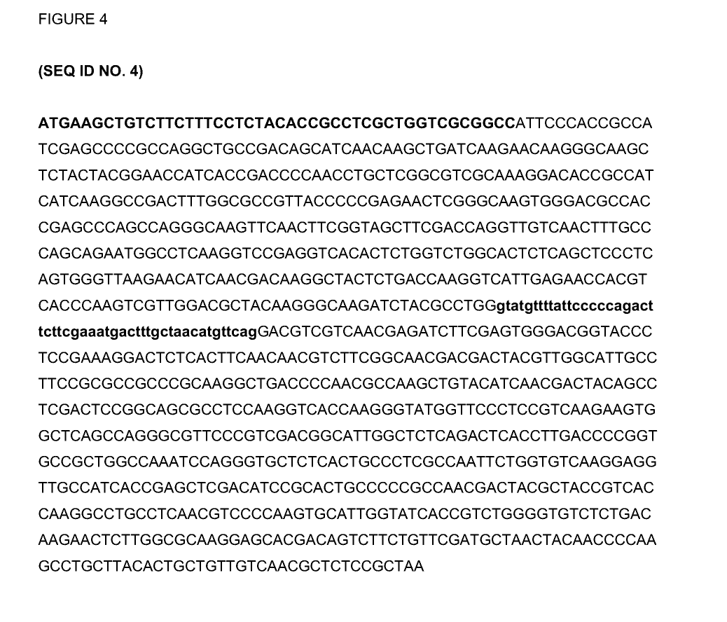

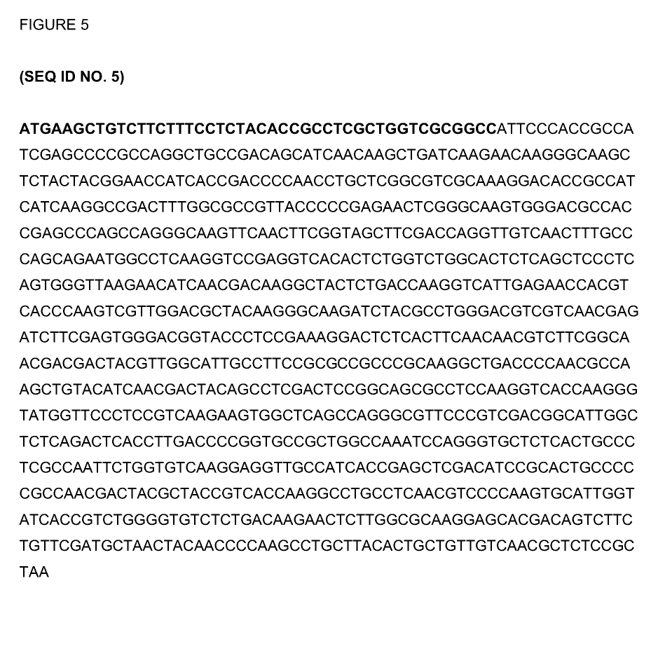

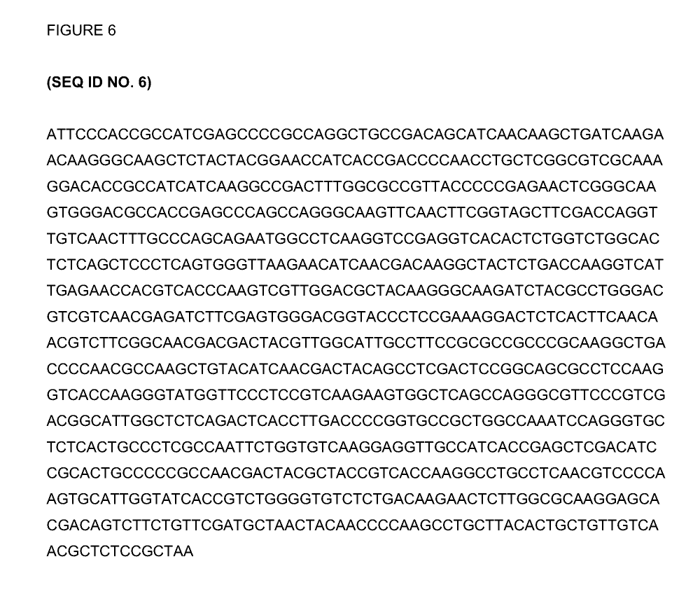

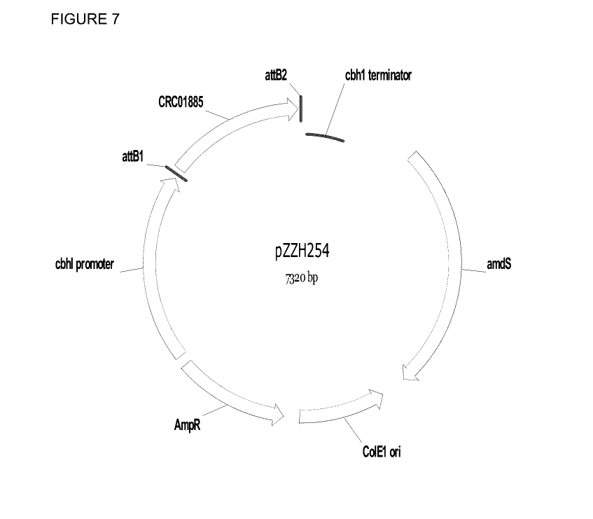

### DETAILED DESCRIPTION

### General Definitions

The headings provided herein are not limitations of the various aspects or embodiments of this disclosure which can be had by reference to the specification as a whole. Accordingly, the terms defined immediately below are more fully defined by reference to the specification as a whole.

Unless defined otherwise, all technical and scientific terms used herein have the same meaning as commonly understood by one of ordinary skill in the art to which this disclosure belongs. Singleton, et al., Dictionary of Microbiology and Molecular Biology, 20 Ed., John Wiley and Sons, New York (1994), and Hale & Marham, The Harper Collins Dictionary of Biology, Harper Perennial, NY (1991) provide one of skill with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The term “consisting essentially of” as used herein means that unspecified components may be present if the characteristics of the claimed composition are thereby not materially affected.

The term “consisting of” means that the proportions of the specific ingredients must total 100%.

The term “comprising” used herein may be amended in some embodiments to refer to consisting essentially of or consisting of (both having a more limited meaning that “comprising”).

In this specification, when the amount of an ingredient is expressed as “% by weight of the flour”, this means the weight in g per 100 g flour (i.e. relative to the flour as 100%). The expression “baker's %” also means the weight in g of a particular ingredient per 100 g of flour.

Amino acids are referred to herein using the name of the amino acid, the three letter abbreviation or the single letter abbreviation.

The term “protein”, as used herein, includes proteins, polypeptides, and peptides.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to understand that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “an enzyme” includes a plurality of such candidate agents, and so forth.

### Process

In one aspect, the process of the present invention comprises comprising the step of contacting a corn-based flour with a xylanase enzyme, such that a xylan-containing material native to the corn is degraded;

wherein the xylanase enzyme is selected from the group consisting of xylanases as defined in (A1), (A2), (B) or (C) above.

In some aspects, a corn-based flour is used as a starting material in the process of the invention. Corn-based flour may be produced by known processes (such as grinding) from corn. For the avoidance of doubt the term “corn” as used herein is synonymous with maize, e.g. Zea mays.

In one embodiment the corn is the sole cereal present in the starting material.

In another embodiment, the corn is present in the starting material as part of a mixture of cereals. In this embodiment, the corn may comprise at least 10% of the cereal mixture, such as at least 20% of the cereal mixture, such as at least 30% of the cereal mixture, such as at least 40% of the cereal mixture, such as at least 50% of the cereal mixture, such as at least 60% of the cereal mixture, such as at least 10% of the cereal mixture, such as at least 70% of the cereal mixture, such as at least 80% of the cereal mixture, such as at least 90% of the cereal mixture, such as at least 95% of the cereal mixture, such as such as at least 97% of the cereal mixture, such as at least 99% of the cereal mixture. In this embodiment, the other cereal may be any cereal typically used as a food. Examples of the other cereal include wheat, rye, barley and oats, especially wheat.

In one embodiment, the corn-based flour is a nixtamalised corn flour. Nixtamalisation is carried out by cooking corn in alkaline solution. The cooked product may then steeped and washed to produce nixtamal, which may then be ground to produce the corn-based flour. The flour may then be mixed with water to produce masa.

In one embodiment the nixtamalisation (alkaline corn cooking) is carried out at a pH of 9 to 11. In one embodiment the corn cooking process is carried out at a pH of 10 to 10.5.

The alkali used for the nixtamalisation (alkaline corn cooking) is not particularly limited providing it is at least partially soluble in water and raises the pH of the solution. Examples of suitable alkalis include alkali metal oxides and hydroxides such as sodium hydroxide and potassium hydroxide, alkaline earth metal oxides and hydroxides such as magnesium hydroxide and calcium hydroxide, alkali metal carbonates and hydrogencarbonates such as sodium carbonate and potassium carbonate, sodium hydrogencarbonate and potassium hydrogencarbonate, and alkaline earth metal carbonates and hydrogencarbonates such as magnesium carbonate, calcium carbonate, magnesium hydrogencarbonate and calcium hydrogencarbonate. A preferred alkali is calcium hydroxide.

The temperature of the nixtamalisation (alkaline corn cooking) is typically from 90 to 100° C., and preferably from 95 to 105° C.

The time of the nixtamalisation (alkaline corn cooking) process generally is for 12 to 24 hours.

In one embodiment, the corn-based flour is produced by grinding the nixtamal which results from the nixtamalisation process.

In one embodiment, the corn-based flour may be produced by grinding non-nixtamalised corn.

In one embodiment, the corn-based flour (suitably a nixtamalised corn-based flour) is mixed with water to produce a masa.

In one embodiment the masa is an alkaline masa. The masa may already be sufficiently alkaline as a result of the nixtamalisation process; alternatively the pH of the masa may be adjusted by using further alkali, such as those defined and exemplified above in relation to the nixtamalisation process. The alkali used to alkalify the masa may be the same or different from the alkali used in the nixtamalisation process.

Suitably, the pH of the alkaline masa is from 9 to 11, preferably from 10 to 10.5.

In one embodiment, the masa is an acidic masa. Such an acidic masa may be produced by contacting the alkaline masa, or the nixtamal, with an acid. The acid used to acidify the masa is not particularly limited providing it is at least partially soluble in water and lowers the pH of the solution. Examples of suitable acids include those commonly used in food production, including acetic acid, citric acid, tartaric acid, malic acid, fumaric acid, and lactic acid. A preferred acid is citric acid.

Additional ingredients may also be present in the masa.

In one embodiment, the masa also includes a preservative, particularly when the masa is an acid masa. Any preservative typically used in food production may be used. Particular classes of preservatives include antimicrobial preservatives, which inhibit the growth of bacteria or fungi, including mould, or they can be antioxidants such as oxygen absorbers, which inhibit the oxidation of food constituents. Common antimicrobial preservatives include sorbic acid and its salts (especially potassium sorbate), benzoic acid and its salts, calcium propionate, sodium nitrite, sulfites (such as sulfur dioxide, sodium hydrogen sulfite and potassium hydrogen sulfite) and disodium EDTA. Common antioxidants include butylated hydroxyanisole (BHA), butylated hydroxytoluene (BHT), tert-butyl-hydroquinone (TBHQ) and propyl gallate. Other preservatives include ethanol and methylchloroisothiazolinone. Preferred preservatives include calcium propionate and potassium sorbate.

In one embodiment, the process of the invention comprises contacting a xylanase enzyme, as defined herein, with the corn-based flour.

In another embodiment, the process of the invention comprises contacting a xylanase enzyme, as defined herein, with the masa.

In another embodiment, the process of the invention comprises contacting a xylanase enzyme, as defined herein, with the corn prior to cooking.

As used herein the term “contacted” refers to the indirect or direct application of the enzyme (or composition comprising the enzyme) of the present invention to (a) the corn-based flour; (b) the masa, and/or (c) the corn prior to cooking. Examples of the application methods which may be used, include, but are not limited to, introducing the enzyme into the corn prior to or after cooking, mixing the enzyme with the corn-based flour, or applying the enzyme to the masa after mixing with the water.

The corn may be contacted with the xylanase enzyme before, during or after cooking.

After preparation, the masa may be allowed to rest. Typically the resting time is from 30 seconds to 1 hour, preferably 5 to 30 minutes, more preferably 10 to 20 minutes. Typically the resting temperature is from 10 to 40° C., and preferably ambient temperature.

In one embodiment the corn flour may be processed to produce a masa (or masa dough), typically by adding water and other ingredients well known to those skilled in the art.

After production masa may then be treated in a number of ways.

In one embodiment the masa is then processed into a masa foodstuff. Various masa foodstuffs exist, examples of which include corn tortilla, soft tortilla, corn chips, tortilla chips, taco shells, tamales, derivatives and mixtures thereof. This process may be carried out using procedures well known to those skilled in the art. In one embodiment the masa dough is then processed into a tortilla.

The masa may be introduced into, for example, a tortilla mould or a tortilla sheeter. This is the traditional end use for the masa. In an alternative, the masa can be dried and milled into a “shelf-stable” flour product. The masa may be reconstituted from the flour product at a later stage and then formed into a food product, such as tortilla.

When the masa is processed to form a tortilla, this is typically carried out using a machine which both forms the raw tortilla and then bakes it to obtain the finished tortilla. Typically, the baking is carried out at 150° C. to 300° C., preferably 200 to 260° C. Typically, the baking time is from 10 seconds to 10 minutes, preferably 20 seconds to 2 minutes, more preferably 30 to 90 seconds.

With regard to industrial implementation, typically masa is sold in the form of the dried masa or is formed into a final food product, such as a tortilla, which is then packed. In both of these aspects, one of the advantages of providing the product in this form is that the end user is free of the need to prepare the nixtamal and masa from the corn constituent. The requirements for labour, energy and processing time for the end use are reduced. Moreover, the product is simple to use.

In another embodiment the corn-based flour is processed into a non-masa product, examples of which include corn bread, corn flakes and corn bugles. This may be carried out by various processes well known to those skilled in the art.

When the corn-based flour undergoes the process of the invention (to produce either a masa or non-masa product), in one embodiment the corn is the sole cereal present in the flour.

In another embodiment, the corn is present in the starting material as part of a mixture of flours from different cereals. In this embodiment, the corn may comprise at least 10% of the cereal in the flour, such as at least 20% of the cereal, such as at least 30% of the cereal, such as at least 40% of the cereal, such as at least 50% of the cereal, such as at least 60% of the cereal, such as at least 10% of the cereal, such as at least 70% of the cereal, such as at least 80% of the cereal, such as at least 90% of the cereal, such as at least 95% of the cereal, such as such as at least 97% of the cereal, such as at least 99% of the cereal. In this embodiment, the other cereal may be any cereal typically used as a food. Examples of the other cereal include wheat, rye, barley and oats, especially wheat. The other cereal may be introduced as a raw cereal at the cooking stage, as outlined above, or may be introduced as a flour with the corn flour before, during or after addition of the xylanase enzyme, and/or introduced as a component of the masa during the mixing with water to form the masa.

### Xylanase Enzyme

In this specification the term “xylanase” when used in isolation is an enzyme capable of degrading the linear polysaccharide beta-1,4-xylan into xylooligosaccharides or xylose. Such enzymes are therefore capable of breaking down hemicellulose, one of the major components of plant cell walls. The term “xylanase” herein is synonymous with “endo-β-1,4-xylanase” (EC 3.2.1.8). Xylanases have been used for many years for the modification of complex carbohydrates derived from plant cell wall material.

Without wishing to be bound by theory, it is believed that the xylanase (as defined below) acts so as to degrade xylan-containing ingredients in the corn. The extent of the degradation is described below.

The xylanases used in the present invention are described below. The xylanases may be used individually (i.e. the stated xylanase is the sole xylanase in of the mixture) or may be mixed in any combination.

### Xylanase A1

In one embodiment the xylanase enzyme is selected from the enzyme designated herein as “Xylanase A1” or “FveXyn4”. This enzyme is described generally in PCT/EP2013/066255, unpublished at the filing date of the present application.

Xylanase A1 (FveXyn4) is defined as a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3, or a polypeptide sequence which comprises SEQ ID No. 1, SEQ ID No. 2, SEQ ID No. 3, with a conservative substitution of at least one of the amino acids, or is encoded by a nucleotide sequence shown herein as SEQ ID No. 4, SEQ ID No. 5 or SEQ ID No. 6, or a nucleotide sequence which can hybridize to SEQ ID No. 4, SEQ ID No. 5 or SEQ ID No. 6, under high stringency conditions, or is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code.

In one embodiment, xylanase A1 comprises (or consists of) a polypeptide sequence shown herein as SEQ ID No. 1, SEQ ID No. 2 or SEQ ID No. 3, or a variant, homologue, fragment or derivative thereof having at least 75% identity (such as at least 80%, 85%, 90%, 95%, 98% or 99% identity) with SEQ ID No. 1, SEQ ID No. 2 or SEQ ID No. 3.

In one embodiment, xylanase A1 comprises (or consists of) a polypeptide sequence which comprises SEQ ID No. 1, SEQ ID No. 2, SEQ ID No. 3, with a conservative substitution of at least one of the amino acids.

In one embodiment, xylanase A1 is encoded by a nucleotide sequence shown herein as SEQ ID No. 4, SEQ ID No. 5 or SEQ ID No. 6, or a nucleotide sequence which can hybridize to SEQ ID No. 4, SEQ ID No. 5 or SEQ ID No. 6, under high stringency conditions.

In one embodiment, xylanase A1 is encoded by a nucleotide sequence which has at least at least 75% identity (such as at least 80%, 85%, 90%, 95% 97.7% at least 98%, 98.5% or 99%) identity with SEQ ID No. 4, SEQ ID No. 5 or SEQ ID No. 6.

In one embodiment, xylanase A1 is encoded by a nucleotide sequence which differs from SEQ ID No. 4 or SEQ ID No. 5 or SEQ ID No. 6 due to the degeneracy of the genetic code.

In one embodiment, xylanase A1 may be obtainable from (or obtained from) a fungus, such as a fungus of the genus Fusarium, particularly the species Fusarium verticilloides.

The xylanase A1 may be part of a preparation having, in addition to its xylanase activity, other side activities. Such side activities may include, for example, amylase, lactase, maltase, protease, lipase and phospholipase activity. Preferably, the xylanase activity of the xylanase A1 preparation comprises at least 50%, such as at least 60%, at least 70%, at least 80%, at least 90%, at least 950%, at least 97%, at least 99%, of the total activity of the enzyme preparation.

### Xylanase A2

In one embodiment the xylanase enzyme is selected from the enzyme designated herein as “Xylanase A2” or “FoxXyn2”. This enzyme is also described generally in PCT/EP2013/066255, unpublished at the filing date of the present application.

Xylanase A2 (FoxXyn2) is defined as a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9, or a polypeptide sequence which comprises SEQ ID No. 7, SEQ ID No. 8, or SEQ ID No. 9 with a conservative substitution of at least one of the amino acids; or is encoded by a nucleotide sequence shown herein as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

In one embodiment, xylanase A2 comprises (or consists of) a polypeptide sequence shown herein as SEQ ID No. 7 SEQ ID No. 8, or SEQ ID No. 9, or a variant, homologue, fragment or derivative thereof having at least 75% identity (such as at least 80%, 85%, 90%, 95%, 98% or 99% identity) with SEQ ID No. 7, SEQ ID No. 8, or SEQ ID No. 9.

In one embodiment, xylanase A2 comprises (or consists of) a polypeptide sequence which comprises SEQ ID No. 7, SEQ ID No. 8, or SEQ ID No. 9 with a conservative substitution of at least one of the amino acids;

In one embodiment, xylanase A2 is encoded by a nucleotide sequence shown herein as SEQ ID No. 11, SEQ ID No. 12, SEQ ID No. 13, SEQ ID No. 14, SEQ ID No. 15 or SEQ ID No. 16.

In one embodiment, xylanase A2 is encoded by a nucleotide sequence which can hybridize to, SEQ ID No. 11, SEQ ID No. 12, SEQ ID No. 13, SEQ ID No. 14, SEQ ID No. 15 or SEQ ID No. 16 under high stringency conditions.

In one embodiment, xylanase A2 is encoded by a nucleotide sequence which has at least 75% identity (such as at least 80%, 85%, 90%, 95% or 98% identity) with SEQ ID No. 11, SEQ ID No. 12, SEQ ID No. 13, SEQ ID No. 14, SEQ ID No. 15 or SEQ ID No. 16.

In one embodiment, xylanase A2 is encoded by a nucleotide sequence which differs from SEQ ID No. 11 or SEQ ID No. 12 or SEQ ID No. 13 or SEQ ID No. 14 or SEQ ID No. 15 or SEQ ID No. 16 due to the degeneracy of the genetic code.

In one embodiment, xylanase A2 may be obtainable from (or obtained from) a fungus, such as a fungus of the genus Fusarium, particularly the species Fusarium oxysporum.

For xylanase A1 and xylanase A2, preferably, the % sequence identity with regard to a polypeptide sequence is determined using SEQ ID No. 3 as the subject sequence in a sequence alignment. In one embodiment, the polypeptide subject sequence is selected from the group consisting of SEQ ID No. 3, SEQ ID No. 1, SEQ ID No. 2, SEQ ID No. 7, SEQ ID No. 8, or SEQ ID No. 9. In a preferred embodiment the polypeptide subject sequence is selected from the mature sequences SEQ ID No. 3, or SEQ ID No. 9.

For xylanase A1 and xylanase A2, preferably, the % sequence identity with regard to a nucleotide sequence is determined using SEQ ID No. 6 as the subject sequence in the sequence alignment. In one embodiment, the subject sequence for nucleotide sequences may be selected from the group consisting of SEQ ID No. 4, SEQ ID No. 5. SEQ ID No. 6, SEQ ID No. 11, SEQ ID No. 12, SEQ ID No. 13, SEQ ID No. 14, SEQ ID No. 15 and SEQ ID No. 16. In a preferred embodiment the subject sequence is sequence SEQ ID No. 6.

The xylanase A2 may be part of a preparation having, in addition to its xylanase activity, other side activities. Such side activities may include, for example, amylase, lactase, maltase, protease, lipase and phospholipase activity. Preferably, the xylanase activity of the xylanase A2 preparation comprises at least 50%, such as at least 60%, at least 70%, at least 80%, at least 90%, at least 950%, at least 97%, at least 99%, of the total activity of the enzyme preparation.

### Xylanase B

In one embodiment the xylanase enzyme is selected from the enzyme designated herein as “Xylanase B” or “AclXyn5”. This enzyme is described generally in PCT/EP2013/066256, unpublished at the filing date of the present application.

Xylanase B (AclXyn5) is defined as a polypeptide as set forth in SEQ ID No. 17 or SEQ ID No. 18 or SEQ ID No. 19; or a variant, fragment, homologue or derivative thereof having at least 85% (suitably at least 90% or at least 95%) identity with SEQ ID No. 17 or SEQ ID No. 18 or SEQ ID No. 19; or encoded by a nucleotide sequence shown herein as SEQ ID No. 20, SEQ ID No. 21 or SEQ ID No. 22, or a nucleotide sequence which can hybridize to the complement of SEQ ID No. 20, SEQ ID No. 21 or SEQ ID No. 22 under high stringency conditions, or is encoded by a nucleotide sequence which has at least 80% (suitably at least 85% or at least 90% or at least 95%) identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code.

In one embodiment, xylanase B comprises a polypeptide as set forth in SEQ ID No. 19 or SEQ ID No. 18 or SEQ ID No. 17; or a variant, homologue or derivative thereof having at least 85% identity (such as at least 90% identity, such as at least 95% identity, such as at least 97% identity, such as at least 98% identity such as at least 99% identity) with SEQ ID No. 19 or SEQ ID No. 18 or SEQ ID No. 17.

In one embodiment, xylanase B comprises a polypeptide encoded by a nucleotide sequence shown herein as SEQ ID No. 22, SEQ ID No. 21 or SEQ ID No. 20.

In one embodiment, xylanase B comprises a polypeptide encoded by a nucleotide sequence which can hybridize to the complement of SEQ ID No. 22, SEQ ID No. 21 or SEQ ID No. 20 under high stringency conditions,

In one embodiment, xylanase B comprises a polypeptide encoded by a nucleotide sequence which has at least 80% identity (such as at least 85% identity, such as at least 90% identity, such as at least 95% identity, such as at least 97% identity, such as at least 98% identity such as at least 99% identity) with SEQ ID No. 22, SEQ ID No. 21 or SEQ ID No. 20.

In one embodiment, xylanase B comprises a polypeptide encoded by a nucleotide sequence which differs from with SEQ ID No. 22, SEQ ID No. 21 or SEQ ID No. 20 due to the degeneracy of the genetic code.

In one embodiment, xylanase B may be obtainable from (or obtained from) a fungus, such as a fungus of the genus Aspergillus, particularly the species Aspergillus clavatus.

The xylanase B may be part of a preparation having, in addition to its xylanase activity, other side activities. Such side activities may include, for example, amylase, lactase, maltase, protease, lipase and phospholipase activity. Preferably, the xylanase activity of the xylanase B preparation comprises at least 50%, such as at least 60%, at least 70%, at least 80%, at least 90%, at least 950%, at least 97%, at least 99%, of the total activity of the enzyme preparation.

### Xylanase C

In one embodiment the xylanase enzyme is selected from the enzyme designated herein as “xylanase C”. Xylanase C is a thermostable xylanase.

In one embodiment, xylanase C comprises a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of (preferably at three or more of, more preferably at least all five of) the following positions 7, 33, 79, 217 and 298, wherein the numbering is based on the amino acid numbering of FveXyn4 (SEQ ID No. 3).

In one embodiment, xylanase C is encoded by a nucleic acid molecule (e.g. an isolated or recombinant nucleic acid molecule) encoding a thermostable xylanase and comprising (or consisting of) a backbone polynucleotide sequence comprising (or consisting of) a nucleotide sequence selected from the group consisting of:
a. a nucleotide sequence shown herein as SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or
b. a nucleotide sequence having at least 70% identity (suitably at least 80%, suitably at least 90%, suitably at least 95%, suitably at least 98%, suitably at least 99% identity) with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or
c. a nucleotide sequence which can hybridize to SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15 under high stringency conditions;
which backbone polynucleotide sequence is modified at two or more of (preferably at three or more, more preferably at least all five of) the codons encoding amino acids 7, 33, 79, 217 and 298 in the encoded polypeptide, wherein the numbering is based on the amino acid numbering of FveXyn4 (SEQ ID No. 3).

In one embodiment, xylanase C is a vector (e.g. a plasmid) or construct comprising (or consisting of) a backbone polynucleotide sequence comprising a nucleotide sequence selected from the group consisting of:
a. a nucleotide sequence shown herein as SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or
b. a nucleotide sequence having at least 70% identity (suitably at least 80%, suitably at least 90%, suitably at least 95%, suitably at least 98%, suitably at least 99% identity) with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or
c. a nucleotide sequence which can hybridize to SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15 under high stringency conditions;
which backbone polynucleotide sequence is modified at two or more of (preferably at three or more, more preferably at least all five of) the codons encoding amino acids 7, 33, 79, 217 and 298 in the encoded polypeptide, wherein the numbering is based on the amino acid numbering of FveXyn4 (SEQ ID No. 3.

In one embodiment, xylanase C is a host cell comprising the nucleic acid according to the present invention or a vector or construct according to the present invention.

In one embodiment, xylanase C is an enzyme having xylanase activity, said enzyme being a GH10 xylanase or a fragment thereof, said enzyme having modifications at two or more (suitably three or more, suitably at least all) of the following positions 7, 33, 79, 217 and 298 wherein the numbering is based on the amino acid numbering of FveXyn4 (SEQ ID No. 3) and said enzyme having increased thermostability compared to a GH10 xylanase which comprises an amino acid sequence which is identical to said enzyme except for said modifications.

In one embodiment, xylanase C is a GH10 xylanase enzyme or a fragment thereof having xylanase activity wherein said GH10 xylanase enzyme comprises a polypeptide having at least 70% (suitably at least 80%, suitably at least 90%, suitably at least 95%, suitably at least 98%, suitably at least 99%) identity to a GH10 xylanase (e.g. a parent GH10 xylanase); and comprises the following amino acids at two or more of (suitably at three or more of, suitably at all of) the positions indicated: 7D; 33V; 79Y, V, F, I, L or M (preferably 79Y, F or V, more preferably Y); 217Q, E, P, D or M (preferably 217Q, E or P, more preferably Q); and 298Y, F or W (preferably Y or F, more preferably Y) wherein the numbering is based on the amino acid numbering of FveXyn4 (SEQ ID No. 3).

In one embodiment, xylanase C is a GH10 xylanase enzyme or a fragment thereof having xylanase activity wherein said GH10 xylanase enzyme comprises a polypeptide having at least 90% (suitably at least 95%, suitably at least 98%, suitably at least 99%) identity to a GH10 xylanase (e.g. a parent or backbone GH10 xylanase); and comprises at the following amino acids at two or more of (suitably at three or more of, suitably at all of) the positions indicated: 7D; 33V; 79Y; 217Q); and 298Y wherein the numbering is based on the amino acid numbering of FveXyn4 (SEQ ID No. 3).

In one embodiment, xylanase C is a GH10 xylanase or a fragment thereof having xylanase activity, wherein said enzyme or fragment thereof has increased thermostability compared with a parent GH10 xylanase enzyme, the parent GH10 xylanase having been modified at, at least, two of the following positions 7, 33, 79, 217 and 298, wherein the numbering is based on the amino acid numbering of FveXyn4 (SEQ ID No. 3).

In one embodiment, xylanase C is an enzyme wherein said enzyme is a GH10 xylanase or a fragment thereof having xylanase activity, wherein said enzyme or fragment thereof has increased thermostability compared with a parent GH10 xylanase enzyme, the parent GH10 xylanase having been modified at, at least, three of the following positions 7, 33, 79, 217 and 298, wherein the numbering is based on the amino acid numbering of FveXyn4 (SEQ ID No. 3).

In one embodiment, xylanase C is a GH10 xylanase or a fragment thereof having xylanase activity, wherein said enzyme or fragment thereof has increased thermostability compared with a parent GH10 xylanase enzyme, the parent GH10 xylanase having been modified at, at least, the following positions 7, 33, 79, 217 and 298, wherein the numbering is based on the amino acid numbering of FveXyn4 (SEQ ID No. 3).

In one embodiment xylanase C comprises at least two of (preferably at least three of) the following modifications:
N7D;
T33V;
K79Y, V, F, I, L or M;
A217Q, E, P, D or M; and
T298Y, For W.

In one embodiment, xylanase C comprises the following amino acids at least two of (preferably at least three of) the positions indicated:
7D;
33V;
79Y, V, F, I, L or M;
217Q, E, P, D or M; and
298Y, F or W.

In one embodiment, xylanase C comprises at least two of (preferably at least three of) the following modifications:
N7D;
T33V;
K79Y, F or V;
A217Q, E or P; and
T298Y or F.

In one embodiment, xylanase C comprises the following amino acids at least two of (preferably at least three of) the positions indicated:
7D;
33V;
79Y, F or V;
217Q, E or P; and
298Y or F.

In one embodiment, xylanase C comprises at least two of (preferably at least three of) the following modifications:
N7D;
T33V;
K79Y;
A217Q; and
T298Y.

In one embodiment, xylanase C comprises the following amino acids at least two of (preferably at least three of) the positions indicated:
7D;
33V;
79Y;
217Q; and
298Y.

In one embodiment, xylanase C comprises at least the following modifications:
N7D;
T33V;
K79Y, V, F, I, L or M;
A217Q, E, P, D or M; and
T298Y, For W.

In one embodiment, xylanase C comprises the following amino acids at the positions indicated:
7D;
33V;
79Y, V, F, I, L or M;
217Q, E, P, D or M; and
298Y, F or W.

In one embodiment In one embodiment, xylanase C comprises at least the following modifications:
N7D;
T33V;
K79Y, F or V;
A217Q, E or P; and
T298Y or F.

In one embodiment xylanase C comprises the following amino acids at the positions indicated:
7D;
33V;
79Y, F or V;
217Q, E or P; and
298Y or F.

In one embodiment, xylanase C comprises at least the following modifications:
N7D;
T33V;
K79Y;
A217Q; and
T298Y.

In one embodiment, xylanase C comprises the following amino acids at the positions indicated:
7D;
33V;
79Y;
217Q; and
298Y.

In one embodiment in addition to being modified at two or more (preferably at three or more, more preferably at all) of positions 7, 33, 79, 217 and 298 xylanase C may be further modified at one or more of the following positions: 25, 57, 62, 64, 89, 103, 115, 147, 181, 193, 219.

In one embodiment in addition to being modified at two or more (preferably at three or more, more preferably at all) of positions 7, 33, 79, 217 and 298 xylanase C may be further modified at two or more of the following positions: 25, 57, 62, 64, 89, 103, 115, 147, 181, 193, 219.

In one embodiment in addition to being modified at two or more (preferably at three or more, more preferably at all) of positions 7, 33, 79, 217 and 298 xylanase C may be further modified at three or more of the following positions: 25, 57, 62, 64, 89, 103, 115, 147, 181, 193, 219.

In one embodiment in addition to being modified at two or more (preferably at three or more, more preferably at all) of positions 7, 33, 79, 217 and 298 xylanase C may be further modified at four or more of the following positions: 25, 57, 62, 64, 89, 103, 115, 147, 181, 193, 219.

In one embodiment in addition to being modified at two or more (preferably at three or more, more preferably at all) of positions 7, 33, 79, 217 and 298 xylanase C may be further modified at five or more of the following positions: 25, 57, 62, 64, 89, 103, 115, 147, 181, 193, 219.

In one embodiment in addition to being modified at two or more (preferably at three or more, more preferably at all) of positions 7, 33, 79, 217 and 298 xylanase C may be further modified at seven or more of the following positions: 25, 57, 62, 64, 89, 103, 115, 147, 181, 193, 219.

In one embodiment in addition to being modified at two or more (preferably at three or more, more preferably at all) of positions 7, 33, 79, 217 and 298 xylanase C may be further modified at nine or more of the following positions: 25, 57, 62, 64, 89, 103, 115, 147, 181, 193, 219.

When xylanase C is further modified at position 25, the modification may be N25P. In other words the amino acid at residue 25 of the GH10 xylanase of the present invention is preferably P.

When xylanase C is further modified at position 57, the modification may be selected from S57Q, T or V (preferably Q). In other words the amino acid at residue 57 of the GH10 xylanase of the present invention is preferably Q, T or V (preferably Q).

When xylanase C is further modified at position 62, the modification may be selected from N62T or S (preferably T). In other words the amino acid at residue 62 of the GH10 xylanase of the present invention is preferably T or S (preferably T).

When xylanase C is further modified at position 64, the modification may be selected from G64T or S (preferably T). In other words the amino acid at residue 64 of the GH10 xylanase of the present invention is preferably T or S (preferably T).

When xylanase C is further modified at position 89, the modification may be selected from S89G, N, Q, L or M (preferably G or Q, more preferably G). In other words the amino acid at residue 89 of the GH10 xylanase of the present invention is preferably G, N, Q, L or M (preferably G or Q, more preferably G).

When xylanase C is further modified at position 103, the modification may be selected from T103M or K (preferably M). In other words the amino acid at residue 103 of the GH10 xylanase of the present invention is preferably M or K (preferably M).

When xylanase C is further modified at position 115, the modification may be selected from V115E or L (preferably L). In other words the amino acid at residue 115 of the GH10 xylanase of the present invention is preferably E or L (preferably L).

When xylanase C is further modified at position 147, the modification may be N147Q. In other words the amino acid at residue 147 of the GH10 xylanase of the present invention is preferably Q.

When xylanase C is further modified at position 181, the modification may be selected from G181Q, A, D or P (preferably Q). In other words the amino acid at residue 181 of the GH10 xylanase of the present invention is preferably Q, A, D or P (preferably Q).

When xylanase C is further modified at position 193, the modification may be selected from S193Y or N (preferably Y). In other words the amino acid at residue 193 of the GH10 xylanase of the present invention is preferably 193Y or N (preferably Y).

When xylanase C is further modified at position 219, the modification may be selected from G219D or P (preferably P). In other words the amino acid at residue 219 of the GH10 xylanase of the present invention is preferably D or P (preferably P).

In one embodiment, xylanase C in addition to comprising modifications at two or more (preferably at three or more, more preferably at all) of positions 7, 33, 79, 217 and 298 further comprises modifications in the following residues: 25 and 89 (preferably N25P and S89G).

In one embodiment, xylanase C in addition to comprising modifications at two or more (preferably at three or more, more preferably at all) of positions 7, 33, 79, 217 and 298 further comprises modifications in the following residues: 57, 62, 64 and 89 (preferably S57Q, N62T, G64T and S89G).

In one embodiment, xylanase C in addition to comprising modifications at two or more (preferably at three or more, more preferably at all) of positions 7, 33, 79, 217 and 298 further comprises modifications in the following residues: 25, 57, 62, 64, 103, 115, 147, 181, 193 and 219 (preferably N25P, S57Q, N62T, G64T T103M, V115L, N147Q, G181Q, S193Y and G219P).

In one embodiment, xylanase C in addition to comprising modifications at two or more (preferably at three or more, more preferably at all) of positions 7, 33, 79, 217 and 298 further comprises modifications in the following residues: 25, 57, 62, 89, 103, 115, 147, 181, 193 and 219 (preferably N25P, S57Q, N62T, S89G, T103M, V115L, N147Q, G181Q, S193Y, G219P and T298Y.

In one embodiment xylanase C in addition to comprising modifications at two or more (preferably at three or more, more preferably at all) of positions 7, 33, 79, 217 and 298 further comprises modifications in the following residues: 25, 89 and 64 (preferably N25P, S89G, G64T)

In one embodiment, xylanase C may comprise the following amino acids at the positions indicated:
a. 7D, 25P, 33V, 64T, 79Y, 89G, 217Q and 298Y;
b. 7D, 25P, 33V, 79Y, 89G, 217Q and 298Y;
c. 7D, 25P, 33V, 57Q, 62T, 64T, 79Y, 103M, 115L, 147Q, 181Q, 193Y, 217Q, 219P and 298Y;
d. 7D, 25P, 33V, 57Q, 62T, 79Y, 89G, 103M, 115L, 147Q, 181Q, 193Y, 217Q, 219P and 298Y;
e. 7D, 33V, 57Q, 62T, 64T, 79Y, 89G, 217Q and 298Y;
f. 79F_217Q_and 298F;
g. 7D,_33V,_217Q_and 298F;
h. 7D,_79F and 298F;
i. 33V, 79F and_217Q;
j. 7D, 33V and_298 Y;
k. 33V,_217Q_and 298Y;
l. 7D,_217Q and_298F;
m. 7D,_33V and 217Q;
n. 79F and 298F;
o. 7D and 79F;
p. 33V_and 79F;
q. 33V and_298 Y;
r. 7D_and 33V; or
s. 33V and_A217Q.

In one embodiment, xylanase C may comprise the following amino acids at the positions indicated:
a. 7D, 25P, 33V, 64T, 79Y, 89G, 217Q and 298Y;
b. 7D, 25P, 33V, 79Y, 89G, 217Q and 298Y;
c. 7D, 25P, 33V, 57Q, 62T, 64T, 79Y, 103M, 115L, 147Q, 181Q, 193Y, 217Q, 219P and 298Y;
d. 7D, 25P, 33V, 57Q, 62T, 79Y, 89G, 103M, 115L, 147Q, 181Q, 193Y, 217Q, 219P and 298Y;
e. 7D, 33V, 57Q, 62T, 64T, 79Y, 89G, 217Q and 298Y;

In one embodiment, xylanase C may comprise the following modifications:
a. N7D, N25P, T33V, G64T, K79Y, S89G, A217Q and T298Y;
b. N7D, N25P, T33V, K79Y, S89G, A217Q and T298Y;
c. N7D, N25P, T33V, S57Q, N62T, G64T, K79Y, T103M, V115L, N147Q, G181Q, S193Y, A217Q, G219P and T298Y;
d. N7D, N25P, T33V, S57Q, N62T, K79Y, S89G, T103M, V115L, N147Q, G181Q, S193Y, A217Q, G219P and T298Y;
e. N7D, T33V, S57Q, N62T, G64T, K79Y, S89G, A217Q and T298Y;
f. K79F_A217Q_T298F;
g. N7D_T33V_A217Q_T298F;
h. N7D_K79F_T298F;
i. T33V_K79F_A217Q;
j. N7D_T33V_T298Y;
k. T33V_A217Q_T298Y;
l. N7D_A217Q_T298F;
m. N7D_T33V_A217Q;
n. K79F_T298F;
o. N7D_K79F;
p. T33V_K79F;
q. T33V_T298Y;
r. N7D_T33V; or
s. T33V_A217Q.

In one embodiment, xylanase C may comprise the following modifications:
a. N7D, N25P, T33V, G64T, K79Y, S89G, A217Q and T298Y;
b. N7D, N25P, T33V, K79Y, S89G, A217Q and T298Y;
c. N7D, N25P, T33V, S57Q, N62T, G64T, K79Y, T103M, V115L, N147Q, G181Q, S193Y, A217Q, G219P and T298Y;
d. N7D, N25P, T33V, S57Q, N62T, K79Y, S89G, T103M, V115L, N147Q, G181Q, S193Y, A217Q, G219P and T298Y;
e. N7D, T33V, S57Q, N62T, G64T, K79Y, S89G, A217Q and T298Y;

In one embodiment xylanase C has a backbone amino acid sequence (before modification) which comprises (or consists of) an amino acid sequence selected from the group consisting of SEQ ID No. 3, SEQ ID No. 1, SEQ ID No. 2, SEQ ID No. 9, SEQ ID No 7, SEQ ID No. 8, or SEQ ID No. 44; or an amino acid sequence having at least 70% identity (suitably at least 80%, suitably at least 90%, suitably at least 95%, suitably at least 98%, suitably at least 99% identity) with SEQ ID No. 3, SEQ ID No. 1, SEQ ID No. 2, SEQ ID No. 9, SEQ ID No 7, SEQ ID No. 8, or SEQ ID No. 44; or an amino acid sequence encoded by a nucleotide sequence comprising the nucleotide sequence shown herein as SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or an amino acid sequence encoded by a nucleotide sequence comprising a nucleotide sequence having at least 70% identity (suitably at least 80%, suitably at least 90%, suitably at least 95%, suitably at least 98%, suitably at least 99% identity) with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or an amino acid sequence encoded by a nucleotide sequence which can hybridize to SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15 under high stringency conditions.

The term “parent” means a xylanase, preferably a GH10 xylanase, to which an alteration is made to produce a modified enzyme of the present invention. In one embodiment the parent enzyme is a GH10 xylanase. Suitably the parent enzyme may be a naturally occurring (wild-type) polypeptide or a variant or fragment thereof. In a preferred embodiment the parent enzyme is a naturally occurring (wild-type polypeptide).

Suitably xylanase C comprises (or consists essentially of, or consists of) an amino acid sequence which is identical or substantially identical to said parent enzyme except for a modification at two or more (preferably at three or more, more preferably at least all five of) the following positions 7, 33, 79, 217 and 298, wherein the numbering is based on the amino acid numbering of FveXyn4 (SEQ ID No. 3).

In some embodiments xylanase C comprises (or consists essentially of, or consists of) an amino acid sequence which is identical or substantially identical to said parent enzyme except for a modification at two or more (preferably at three or more, more preferably at least all five of) the following positions 7, 33, 79, 217 and 298, as well as at one or more of the following positions 25, 57, 62, 64, 89, 103, 115, 147, 181, 193, 219, wherein the numbering is based on the amino acid numbering of FveXyn4 (SEQ ID No. 1).

Xylanase C suitably has about at least 90% sequence identity (preferably at least 93%, suitably at least 97%, suitably at least 99% sequence identity to the parent enzyme.

The term “backbone” as used herein means a polypeptide sequence that is a GH10 xylanase polypeptide, which is modified to comprise the following amino acids at two or more (preferably at three or more, more preferably at all) of the positions indicated: 7D; 33V; 79Y, V, F, I, L or M (preferably 79Y, F or V, more preferably Y); 217Q, E, P, D or M (preferably 217Q, E or P, more preferably Q); and 298Y, F or W (preferably Y or F, more preferably Y) wherein the numbering is based on the amino acid numbering of FveXyn4 (SEQ ID No. 3).

Xylanase C preferably comprises a polypeptide having at least 70% (suitably at least 80%, suitably at least 90%, suitably at least 95%, suitably at least 98%, suitably at least 99%) identity to a GH10 xylanase (e.g. a parent or backbone GH10 xylanase); and comprises the following amino acids at two or more (preferably at three or more, more preferably at all) of the positions indicated: 7D; 33V; 79Y, V, F, I, L or M (preferably 79Y, F or V, more preferably Y); 217Q, E, P, D or M (preferably 217Q, E or P, more preferably Q); and 298Y, F or W (preferably Y or F, more preferably Y) wherein the numbering is based on the amino acid numbering of FveXyn4 (SEQ ID No. 3).

Xylanase C preferably comprises a polypeptide having at least at least 95% (suitably at least 98%, suitably at least 99%) identity to a GH10 xylanase (e.g. a parent or backbone GH10 xylanase); and comprises the following amino acids at two or more (preferably at three or more, more preferably at all) of the positions indicated: 7D; 33V; 79Y; 217Q); and 298Y wherein the numbering is based on the amino acid numbering of FveXyn4 (SEQ ID No. 1).

In one embodiment the parent or backbone GH10 xylanase (before modification) is:
a. a xylanase comprising an amino acid sequence selected from the group consisting of SEQ ID No. 3, SEQ ID No. 1, SEQ ID No. 2, SEQ ID No. 9, SEQ ID No 7, SEQ ID No. 8, or SEQ ID No. 44; or
b. a xylanase enzyme comprising an amino acid sequence having at least 70% identity (suitably at least 80%, suitably at least 90%, suitably at least 95%, suitably at least 98%, suitably at least 99% identity) with SEQ ID No. 3, SEQ ID No. 1, SEQ ID No. 2, SEQ ID No. 9, SEQ ID No 7, SEQ ID No. 8, or SEQ ID No. 44; or
c. a xylanase enzyme encoded by a nucleotide sequence comprising the nucleotide sequence shown herein as SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or
d. a xylanase enzyme encoded by a nucleotide sequence comprising a nucleotide sequence having at least 70% identity (suitably at least 80%, suitably at least 90%, suitably at least 95%, suitably at least 98%, suitably at least 99% identity) with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15; or
e. a xylanase enzyme encoded by a nucleotide sequence which can hybridize to SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15 under high stringency conditions.

In one embodiment the parent or backbone amino acid sequence has at least 80% identity with SEQ ID No. 3, SEQ ID No. 1, SEQ ID No. 2, SEQ ID No. 9, SEQ ID No 7, SEQ ID No. 8, or SEQ ID No. 44.

In one embodiment the parent or backbone amino acid sequence has at least 90% identity with SEQ ID No. 3, SEQ ID No. 1, SEQ ID No. 2, SEQ ID No. 9, SEQ ID No 7, SEQ ID No. 8, or SEQ ID No. 44.

In one embodiment the parent or backbone amino acid sequence has at least 95% identity with SEQ ID No. 3, SEQ ID No. 1, SEQ ID No. 2, SEQ ID No. 9, SEQ ID No 7, SEQ ID No. 8, or SEQ ID No. 44.

In one embodiment the parent or backbone amino acid sequence has at least 98% identity with SEQ ID No. 3, SEQ ID No. 1, SEQ ID No. 2, SEQ ID No. 9, SEQ ID No 7, SEQ ID No. 8, or SEQ ID No. 44.

In one embodiment the parent or backbone xylanase enzyme may be encoded by a nucleotide sequence comprising a nucleotide sequence having at least 80% identity with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15.

In one embodiment the parent or backbone xylanase enzyme may be encoded by a nucleotide sequence comprising a nucleotide sequence having at least 90% identity with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15.

In one embodiment the parent or backbone xylanase enzyme may be encoded by a nucleotide sequence comprising a nucleotide sequence having at least 95% identity with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15.

In one embodiment the parent or backbone xylanase enzyme may be encoded by a nucleotide sequence comprising a nucleotide sequence having at least 98% identity with SEQ ID No. 6, SEQ ID No. 4, SEQ ID No. 5, SEQ ID No. 13, SEQ ID No. 11, SEQ ID No. 12, SEQ ID No. 16, SEQ ID No 14 or SEQ ID No. 15.

Suitably, the parent or backbone GH10 xylanase may be obtainable (suitably obtained) from a Fusarium organism.

Suitably the parent or backbone xylanase is an endo-1,4-β-d-xylanase.

The modified xylanase or GH10 xylanase according to the present invention is preferably an endo-1,4-β-d-xylanase.

In a preferred embodiment, the enzyme having xylanase activity, e.g. the GH10 xylanase enzyme (such as the modified GH10 xylanase enzyme) or a fragment thereof according to the present invention has a Tm value of more than 70° C. (preferably more than 75° C.), wherein the Tm value is measured as the temperature at which 50% residual activity is obtained after 10 min incubation.

The thermostability of a xylanase (e.g. xylanase C) in accordance with the present invention may be determined using the “Assay for measurement of thermostability” (see below).

### Assay for Measurement of Thermostability

The thermal denaturation profiles of the FveXyn4 variants was measured by diluting and pre-incubating the enzyme samples in 25 mM MES buffer, pH 6.0 for 10 min at varying temperatures (63, 65.5, 66.7, 68.2, 70.6, 73.5, 76, 76.5, 76.8, 79.7, 81.9, 83.5, 84.6, and 85° C., respectively) and subsequently measuring the residual activity by the xylanase activity method described in Example 1. Activity measured without pre-incubation was set to 100% and the residual activity of each variant at each temperature was calculated as relative to this. Tm value is calculated from the thermal denaturation profiles as the temperature at which 50% residual activity is obtained.

In one embodiment, an enzyme is considered to be thermostable in accordance with the present invention if it has a Tm value of more than 70° C., wherein the Tm value is the temperature at which 50% residual activity is obtained after 10 min incubation. This Tm value may be measured in accordance with the assay for measurement of thermostability as taught herein.

In one embodiment, an enzyme is considered to be thermostable in accordance with the present invention if it has a Tm value of more than 76° C., wherein the Tm value is the temperature at which 50% residual activity is obtained after 10 min incubation.

This Tm value may be measured in accordance with the assay for measurement of thermostability as taught herein.

In one embodiment, an enzyme is considered to be thermostable in accordance with the present invention if it has a Tm value of more than 85° C., wherein the Tm value is the temperature at which 50% residual activity is obtained after 10 min incubation. This Tm value may be measured in accordance with the assay for measurement of thermostability as taught herein.

Preferably, the enzyme having xylanase activity, e.g. the GH10 xylanase enzyme (such as the modified GH10 xylanase enzyme) or a fragment thereof according to the present invention (or composition comprising same) can withstand a heat treatment (e.g. during the pelleting process for example) of up to about 70° C.; e.g. up to 75° C., e.g. up to 76° C., e.g. up to about 85° C.; e.g. or up to about 95° C. The heat treatment may be performed for up to about 1 minute; up to about 5 minutes; up to about 10 minutes; up to about 30 minutes; up to about 60 minutes. To withstand such heat treatment means that at least about 50% of the enzyme that was present/active in the additive before heating to the specified temperature is still present/active after it cools to room temperature. Preferably, at least about 80% of the enzyme that is present and active in the additive before heating to the specified temperature is still present and active after it cools to room temperature.

The term “thermostability” is the ability of an enzyme to resist irreversible inactivation (usually by denaturation) at a relatively high temperature. This means that the enzyme retains a specified amount of enzymatic activity after exposure to an identified temperature over a given period of time.

There are many ways of measuring thermostabiliy. By way of example, enzyme samples maybe incubated without substrate for a defined period of time (e.g. 10 min or 1 to 30 min) at an elevated temperature compared to the temperature at which the enzyme is stable for a longer time (days). Following the incubation at elevated temperature the enzyme sample is assayed for residual activity at the permissive temperature of e.g. 30° C. (alternatively 25-50° C. or even up to 70° C.). Residual activity is calculated as relative to a sample of the enzyme that has not been incubated at the elevated temperature.

Thermostability can also be measured as enzyme inactivation as function of temperature. Here enzyme samples are incubated without substrate for a defined period of time (e.g. 10 min or 1 to 30 min) at various temperatures and following incubation assayed for residual activity at the permissive temperature of e.g. 30° C. (alternatively 25-70° C. or even higher). Residual activity at each temperature is calculated as relative to a sample of the enzyme that has not been incubated at the elevated temperature. The resulting thermal denaturation profile (temperature versus residual activity) can be used to calculate the temperature at which 50% residual activity is obtained. This value is defined as the Tm value.

Even further, thermostability can be measured as enzyme inactivation as function of time. Here enzyme samples are incubated without substrate at a defined elevated temperature (e.g. 76° C.) for various time periods (e.g. between 10 sec and 30 min) and following incubation assayed for residual activity at the permissive temperature of e.g. 30° C. (alternatively 25-70° C. or even higher). Residual activity at each temperature is calculated as relative to an enzyme sample that has not been incubated at the elevated temperature. The resulting inactivation profile (time versus residual activity) can be used to calculate the time at which 50% residual activity is obtained. This is usually given as T1/2.

These are examples of how to measure thermostability. Thermostability can also be measured by other methods. Preferably thermostability is assessed by use of the “Assay for measurement of thermostability” as taught herein.

In contradistinction to thermostability, thermoactivity is enzyme activity as a function of temperature. To determine thermoactivity enzyme samples may be incubated (assayed) for the period of time defined by the assay at various temperatures in the presence of substrate. Enzyme activity is obtained during or immediately after incubation as defined by the assay (e.g. reading an OD-value which reflects the amount of formed reaction product). The temperature at which the highest activity is obtained is the temperature optimum of the enzyme at the given assay conditions. The activity obtained at each temperature can be calculated relative to the activity obtained at optimum temperature. This will provide a temperature profile for the enzyme at the given assay conditions.

In the present application thermostability is not the same as thermoactivity.

In a preferred embodiment, xylanase C comprises one of the amino acid sequences shown herein as SEQ ID No. 39, SEQ ID No. 40, SEQ ID No. 41, SEQ ID No. 42, or SEQ ID No. 43, or a fragment thereof having xylanase activity.

In one embodiment the modifications in the backbone polynucleotide sequence are such to render the above detailed modifications in the encoded amino acid sequence:

The methods of the present invention are suitable to render the modifications as taught above in the polynucleotide or amino acid sequence.

The xylanase C may have in addition to its xylanase activity other side activities. Such side activities may include, for example, amylase, lactase, maltase, protease, lipase and phospholipase activity. Preferably, the xylanase activity of the xylanase A1 comprises at least 50%, such as at least 60%, at least 70%, at least 80%, at least 90%, at least 95%, at least 97%, at least 99%, of the total activity of the enzyme.

In one aspect of the invention, the xylanase used in the invention is a xylanase of Glycoside Hydrolyase (GH) Family 10. The term “of Glycoside Hydrolyase (GH) Family 10” means that the xylanase in question is or can be classified in the GH family 10.

Protein similarity searches (e.g. protein blast at http://blast.ncbi.nlm.nih.gov/Blast.cgi?CMD=Web&PAGE_TYPE=BlastHome) may determine whether an unknown sequence falls under the term of a GH10 xylanase family member, particularly the GH families may be categorised based on sequence homology in key regions. In addition or alternatively, to determine whether an unknown protein sequence is a xylanase protein within the GH10 family, the evaluation can be done, not only on sequence similarity/homology/identity, but also on 3D structure similarity. The classification of GH-families is often based on the 3D fold. Software that will predict the 3D fold of an unknown protein sequence is HHpred (http://toolkit.tuebingen.mpg.de/hhpred). The power of this software for protein structure prediction relies on identifying homologous sequences with known structure to be used as template. This works so well because structures diverge much more slowly than primary sequences. Proteins of the same family may have very similar structures even when their sequences have diverged beyond recognition.

In practice, an unknown sequence can be pasted into the software (http://toolkit.tuebingen.mpg.de/hhpred) in FASTA format. Having done this, the search can be submitted. The output of the search will show a list of sequences with known 3D structures. To confirm that the unknown sequence indeed is a GH10 xylanase, GH10 xylanases may be found within the list of homologues having a probability of >90. Not all proteins identified as homologues will be characterised as GH10 xylanases, but some will. The latter proteins are proteins with a known structure and biochemically characterisation identifying them as xylanases. The former have not been biochemically characterised as GH10 xylanases. Several references describes this protocol such as Söding J. (2005) Protein homology detection by HMM-HMM comparison—Bioinformatics 21, 951-960 (doi:10.1093/bioinformatics/bti125) and Soding J, Biegert A, and Lupas A N. (2005) The HHpred interactive server for protein homology detection and structure prediction—Nucleic Acids Research 33, W244-W248 (Web Server issue) (doi:10.1093/nar/gki40).

According to the Cazy site (http://www.cazy.org/), Family 10 glycoside hydrolases can be characterised as follows:

Known Activities: endo-1,4-β-xylanase (EC 3.2.1.8); endo-1,3-β-xylanase (EC 3.2.1.32); tomatinase (EC 3.2.1.-)

### Mechanism: Retaining

### Clan: GH-A

Catalytic Nucleophile/Base: Glu (experimental)
Catalytic Proton Donor: Glu (experimental)

### 3D Structure Status: (β/α)₈

The GH10 xylanase used in the present invention may have a catalytic domain with molecular weights in the range of 32-39 kDa. The structure of the catalytic domain of the GH10 xylanase of the present invention consists of an eightfold β/α barrel (Harris et al 1996—Acta. Crystallog. Sec. D 52, 393-401).

Three-dimensional structures are available for a large number of Family GH10 enzymes, the first solved being those of the Streptomyces lividans xylanase A (Derewenda et al, J Biol Chem 1994 Aug. 19; 269(33) 20811-4), the C. fimi endo-glycanase Cex (White et al Biochemistry 1994 Oct. 25; 33(42) 12546-52), and the Cellvibrio japonicus Xyn10A (previously Pseudomonas fluorescens subsp. xylanase A) (Harris et al, Structure 1994 Nov. 15; 2(11) 1107-16.). As members of Clan GHA they have a classical (α/β)₈ TIM barrel fold with the two key active site glutamic acids located at the C-terminal ends of beta-strands 4 (acid/base) and 7 (nucleophile) (Henrissat et al, Proc Natl Acad Sci USA 1995 Jul. 18; 92(15) 7090-4).

The term “GH10 xylanase” as used herein means a polypeptide having xylanase activity and having a (α/β)₈ TIM barrel fold with the two key active site glutamic acids located at the C-terminal ends of beta-strands 4 (acid/base) and 7 (nucleophile).

The backbone (or parent) xylanase enzyme used herein may be referred to as FveXyn4 or FoxXyn 2 (these terms refer to the active proteins, e.g. the mature proteins).

In one embodiment preferably the xylanase is a fungal xylanase.

The enzyme having xylanase activity, e.g. the GH10 xylanase enzyme (such as the modified GH10 xylanase enzyme) or a fragment thereof according to the present invention and/or parent enzyme is a GH10 xylanase.

In one embodiment preferably the enzyme having xylanase activity, e.g. the GH10 xylanase enzyme (such as the modified GH10 xylanase enzyme) or a fragment thereof according to the present invention (and/or parent xylanase) is a fungal GH10 xylanase.

In one embodiment preferably the enzyme having xylanase activity, e.g. the GH10 xylanase enzyme (such as the modified GH10 xylanase enzyme) or a fragment thereof according to the present invention (and/or parent xylanase) is an endoxylanase, e.g. an endo-1,4-β-d-xylanase. The classification for an endo-1,4-β-d-xylanase is E.C. 3.2.1.8.

The term “fragment thereof” as used herein means an active fragment. In other words the fragment is one which has xylanase activity. Suitably the fragment may have the same xylanase activity as the full length modified GH10 xylanase enzyme from which the fragment is derived. Alternatively, the fragment may have a modified activity (e.g. enhanced specificity, specific activity, pH or temperature profile) compared with the full length modified GH10 xylanase enzyme from which the fragment is derived. In addition the fragment must retain the thermostable properties of the modified GH10 xylanase enzyme of which it is a fragment.

In one embodiment the fragment is at least 60% of the full length of the modified GH10 xylanase enzyme from which the fragment is derived.

In one embodiment the fragment is at least 75% of the full length of the modified GH10 xylanase enzyme from which the fragment is derived.

In one embodiment the fragment is at least 85% of the full length of the modified GH10 xylanase enzyme from which the fragment is derived.

In one embodiment the fragment is at least 95% of the full length of the modified GH10 xylanase enzyme from which the fragment is derived.

In one embodiment the fragment is at least 98% of the full length of the modified GH10 xylanase enzyme from which the fragment is derived.

In one embodiment the fragment is a fragment of one or more of the sequences selected from the group consisting of SEQ ID No. 39, SEQ ID No. 40, SEQ ID No. 41, SEQ ID No.42, or SEQ ID No. 43.

In one embodiment the enzyme having xylanase activity, e.g. the GH10 xylanase enzyme (such as the modified GH10 xylanase enzyme) or a fragment, thereof according to the present invention a) comprises one of the amino acid sequences shown herein as SEQ ID No. 39, SEQ ID No. 40, SEQ ID No. 41, SEQ ID No.42, or SEQ ID No. 43, or b) comprises an amino acid sequence which is at least 96%, preferably at least 98.5%, identical with the amino acid sequences shown herein as SEQ ID No. 39, SEQ ID No. 40, SEQ ID No. 41, SEQ ID No.42, or SEQ ID No. 43 so long as the amino acids at positions 7, 33, 79, 217 and 298 are identical with those shown in SEQ ID No. 39, SEQ ID No. 40, SEQ ID No. 41, SEQ ID No.42, or SEQ ID No. 43.

In one embodiment the present invention provides a nucleic acid molecule according to the present invention or a vector or construct comprising same, wherein the nucleotide sequence is selected from the group consisting of: SEQ ID No. 29, SEQ ID No. 30, SEQ ID No. 31, SEQ ID No. 32, SEQ ID No. 33, SEQ ID No. 34. SEQ ID No. 35, SEQ ID No.36, SEQ ID No. 37 and SEQ ID No. 38; or a nucleotide sequence which is at least 96%, preferably 98.5%, identical with the nucleotide sequences shown herein as SEQ ID No. 29, SEQ ID No. 30, SEQ ID No. 31, SEQ ID No. 32, SEQ ID No. 33, SEQ ID No. 34. SEQ ID No. 35, SEQ ID No.36, SEQ ID No. 37 and SEQ ID No. 38 so long as the codons encoding amino acid positions 7, 33, 79, 217 and 298 in the mature protein the same as those of SEQ ID No. 29, SEQ ID No. 30, SEQ ID No. 31, SEQ ID No. 32, SEQ ID No. 33, SEQ ID No. 34. SEQ ID No. 35, SEQ ID No.36, SEQ ID No. 37 and SEQ ID No. 38.

The term “modifying” as used herein means changing or altering. In particular, the term “modifying” as used herein means altering from the naturally occurring. In other words, when modifying the enzyme, one changes the enzyme in such a way that renders the enzyme altered from the parent backbone enzyme. Preferably the modified enzyme does not exist itself in nature. Thus the modified enzyme is a non-naturally-occurring enzyme.

The term “modified” as used herein means altered, e.g. from its naturally occurring form. The modified enzymes according to the present invention are preferably not naturally occurring enzymes or naturally occurring variants. In other words, the modified enzymes according to the present invention are preferably modified enzymes that have not been found in nature. The modified enzymes of the present invention have preferably not occurred spontaneously.

In some embodiments the enzyme having xylanase activity, e.g. the GH10 xylanase enzyme (such as the modified GH10 xylanase enzyme) or a fragment thereof of the present invention is prepared by modifying a parent enzyme or a backbone enzyme. However in other embodiments the enzyme having xylanase activity, e.g. the GH10 xylanase enzyme (such as the modified GH10 xylanase enzyme) or a fragment thereof of the present invention is prepared without modifying a parent enzyme or a backbone enzyme, e.g. it may be prepared synthetically. The term “modified xylanase” or “modified GH10 xylanase” as used herein does not dictate that the xylanase has been prepared by mutating a parent enzyme. The modified xylanase may suitably have been prepared by other means, e.g. synthetically.

### Xylan-Containing Material

The xylanases used in the present invention may be used to degrade any xylan-containing material. The term “breakdown” or “degrade” is synonymous with hydrolysis. In one embodiment the xylan-containing material is any plant material comprising arabinoxylan.

In one embodiment, the xylanase completely degrades the xylan-containing material into its constituent xylose units.

In one embodiment, the xylanase partially degrades the xylan-containing material into a mixture of xylose monosaccharide units and oligosaccharides and/or polysaccharides containing xylose units. In this specification the term “oligosaccharide” means a carbohydrate containing 2 to 10 monosaccharide units. In this specification the term “polysaccharide” means a carbohydrate containing more than 10, such as 10 to 100,000, such as 50 to 50,000, such as 100 to 10,000, such as 950 to 2000, monosaccharide units.

In a yet further embodiment the xylan-containing material may be a cereal flour (e.g. corn flour, wheat, oat, rye or barley flour), especially corn flour.

### Dosage

The dosage of the xylanase enzyme varies depending on the type of enzyme used, the conditions under which the corn product is cooked, any additional ingredients present, and the intended product. The dosages expressed below are in terms of the enzyme product.

Typically, the xylanase dosage may be from 0.001 to 5 mg/kg corn flour, preferably 0.01 to 2 mg/kg corn-based flour.

In one embodiment, when the xylanase is xylanase A1, the xylanase dosage is typically 0.04 to 0.64 mg/kg of corn-based flour, preferably 0.08 to 0.32 mg/kg of corn-based flour. Such a dosage is particularly preferred when the masa, and/or the masa foodstuff (preferably a tortilla), is produced under acidic conditions.

In one embodiment, when the xylanase is xylanase A1, the xylanase dosage is typically 0.08 to 1.28 mg/kg of corn-based flour preferably 0.16 to 0.64 mg/kg corn-based flour. Such a dosage is particularly preferred when the masa, and/or the masa foodstuff (preferably tortilla), is produced under alkaline conditions.

When the xylanase is xylanase A2 (as defined herein) or xylanase C, the typical and preferred doses may be as detailed above for xylanase A1.

In one embodiment, when the xylanase is xylanase B, the xylanase dosage is typically 0.04 to 0.60 mg/kg of corn-based flour, preferably 0.08 to 0.3 mg/kg of corn-based flour. Such a dosage is particularly preferred when the masa, and/or the masa foodstuff (preferably tortilla), is produced under acid conditions.

In one embodiment, when the xylanase is xylanase B, the xylanase dosage is typically 0.08 to 1.2 mg/kg corn-based flour, preferably 0.15 to 0.6 mg/kg corn-based flour. Such a dosage is particularly preferred when the masa, and/or the tortilla, is produced under alkaline conditions.

Typically, the xylanase dosage may be from 10 to 20000 Units of xylanase activity (XU), preferably 50 to 10000 Units, per kg of corn-based flour.

In one embodiment, when the xylanase is xylanase A1, the xylanase dosage is typically 100 to 2000 Units of xylanase activity (XU), preferably 200 to 1000 Units per kg of corn-based flour. Such a dosage is particularly preferred when the masa, and/or the masa foodstuff (preferably tortilla), is produced under acidic conditions.

In one embodiment, when the xylanase is xylanase A1, the xylanase dosage is typically 200 to 4000 Units of xylanase activity (XU), preferably 400 to 2000 Units, per kg of corn-based flour. Such a dosage is particularly preferred when the masa, and/or the masa foodstuff (preferably tortilla), is produced under alkaline conditions.

In one embodiment, when the xylanase is xylanase B, the xylanase dosage is typically 200 to 3200 Units of xylanase activity (XU), preferably 400 to 1600 Units, per kg of corn-based flour. Such a dosage is particularly preferred when the masa, and/or the masa foodstuff (preferably tortilla), is produced under acidic conditions.

In one embodiment, when the xylanase is xylanase B, the xylanase dosage is typically 400 to 6400 Units of xylanase activity (XU), preferably 800 to 3200 Units, per kg of corn-based flour. Such a dosage is particularly preferred when the masa, and/or the masas foodstuff (preferably tortilla), is produced under alkaline conditions.

The xylanase activity is expressed in xylanase units (XU) measured at pH 5.0 with AZCL-arabinoxylan (azurine-crosslinked wheat arabinoxylan, Xylazyme tablets, Megazyme) as substrate. Hydrolysis by endo-(1-4)-β-D-xylanase (xylanase) produces water soluble dyed fragments, and the rate of release of these (increase in absorbance at 590 nm) can be related directly to enzyme activity. The xylanase units (XU) are determined relatively to an enzyme standard (Danisco xylanase, available from DuPont Industrial Biosciences) at standard reaction conditions, which are 40 C, 5 min reaction time in McIlvaine buffer, pH 5.0.

The xylanase activity of the standard enzyme is determined as amount of released reducing sugar end groups from an oat-spelt-xylan substrate per min at pH 5.3 and 50° C. The reducing sugar end groups react with 3, 5-dinitrosalicylic acid and formation of the reaction product can be measured as increase in absorbance at 540 nm. The enzyme activity is quantified relative to a xylose standard curve (reducing sugar equivalents). One xylanase unit (XU) is the amount of standard enzyme that releases 0.5 μmol of reducing sugar equivalents per min at pH 5.3 and 50° C.

### Hydrocolloid

In a preferred aspect of the process of the present invention, a hydrocolloid is present in addition to the mixture of corn-based flour and xylanase enzyme. Typically, the hydrocolloid is added to the dried corn flour.

In a preferred aspect the flour of the present invention further comprises a hydrocolloid in addition to the xylanase enzyme.

In a preferred aspect the masa of the present invention further comprises a hydrocolloid.

In a preferred aspect the masa foodstuff further of the present invention comprises a hydrocolloid.

The hydrocolloid is preferably present in an amount of from 0.01% to 4% by weight, preferably 0.2 to 2%, such as 0.4 to 1.2%, especially 0.5 to 1%, by weight of the flour. As indicated in the general definitions above, the term “% by weight of the flour” when defining the amount of the hydrocolloid means the weight of the hydrocolloid in g per 100 g flour (i.e. relative to the flour as 100%).

In one embodiment the hydrocolloid is present as an initial component of the flour. In this embodiment, the hydrocolloid is preferably present in an amount of from 0.01% to 4% by weight, preferably 0.2 to 2%, such as 0.4 to 1.2%, especially 0.5 to 1%, by weight of the flour.

In one embodiment the hydrocolloid is added to the flour. In this embodiment, the hydrocolloid is preferably present in an amount of from 0.01% to 4% by weight, preferably 0.2 to 2%, such as 0.4 to 1.2%, especially 0.5 to 1%, by weight of the flour.

Preferably, the hydrocolloid is selected from carboxymethylcellulose (CMC), carrageenan, guar gum, pectin and mixtures thereof.

More preferably the hydrocolloid is carboxymethylcellulose (CMC). Carboxymethyl cellulose (CMC) or cellulose gum is a cellulose derivative with carboxymethyl groups (—CH₂—COOH) bound to some of the hydroxyl groups of the glucopyranose monomers that make up the cellulose backbone.

Preferably, the viscosity of the CMC is 2000 to 10 000 mPa·s, more preferably 5000-9000 mPa·s.

Preferably, the degree of substitution of the CMC is 0.5 to 1, more preferably 0.7 to 0.85.

In a particularly preferred embodiment the hydrocolloid is GRINDSTED™ CMC MASS 550. This is commercially available from DuPont Nutrition BioSciences ApS.

In one embodiment the carboxymethylcellulose is present as an initial component of the flour. In this embodiment, the carboxymethylcellulose is preferably present in an amount of from 0.01% to 4% by weight, preferably 0.2 to 2%, such as 0.4 to 1.2%, especially 0.5 to 1%, by weight of the flour.

In one embodiment the carboxymethylcellulose is added to the flour. In this embodiment, the carboxymethylcellulose is preferably present in an amount of from 0.01% to 4% by weight, preferably 0.2 to 2%, such as 0.4 to 1.2%, especially 0.5 to 1%, by weight of the flour.

In one embodiment, the xylanase is xylanase A1, the xylanase dosage is typically 0.04 to 0.64 mg/kg of corn-based flour, preferably 0.08 to 0.32 mg/kg of corn-based flour. and the hydrocolloid (preferably carboxymethylcellulose) is present in an amount of from 0.2 to 2%, preferably 0.4 to 1% by weight of the flour. Such a dosage is particularly preferred when the masa and/or the tortilla is produced under acidic conditions.

In one embodiment, the xylanase is xylanase A1, the xylanase dosage is typically 0.08 to 1.28 mg/kg of corn-based flour, preferably 0.16 to 0.64 mg/kg corn-based flour and the hydrocolloid (preferably carboxymethylcellulose) is present in an amount of from 0.2 to 2%, preferably 0.4 to 1% by weight of the flour Such a dosage is particularly preferred when the masa, and/or the masa foodstuff (preferably tortilla) is produced under alkaline conditions.

In one embodiment, the xylanase is xylanase B, the xylanase dosage is typically 0.08 to 1.2 mg/kg corn-based flour, preferably 0.15 to 0.6 mg/kg corn-based flour and the hydrocolloid (preferably carboxymethylcellulose) is present in an amount of from 0.2 to 2%, preferably 0.4 to 1% by weight of the flour. Such a dosage is particularly preferred when the masa and/or the masa foodstuff (preferably tortilla) is produced under acid conditions.

In one embodiment, the xylanase is xylanase B, the xylanase dosage is 0.08 to 1.2 mg/kg corn-based flour, preferably 0.15 to 0.6 mg/kg corn-based flour and the hydrocolloid (preferably carboxymethylcellulose) is present in an amount of from 0.2 to 2%, preferably 0.4 to 1% by weight of the flour. Such a dosage is particularly preferred when the masa and/or the masa foodstuff (preferably tortilla) is produced under alkaline conditions.

In one embodiment, the xylanase is xylanase A1, the xylanase dosage is typically 100 to 2000 Units of xylanase activity (XU), preferably 200 to 1000 Units per kg of corn-based flour and the hydrocolloid (preferably carboxymethylcellulose) is present in an amount of from 0.2 to 2%, preferably 0.4 to 1% by weight of the flour. Such a dosage is particularly preferred when the masa and/or the masa foodstuff (preferably tortilla) is produced under acidic conditions.

In one embodiment, when the xylanase is xylanase A1, the xylanase dosage is typically 200 to 4000 Units of xylanase activity (XU), preferably 400 to 2000 Units, per kg of corn-based flour and the hydrocolloid (preferably carboxymethylcellulose) is present in an amount of from 0.2 to 2%, preferably 0.4 to 1% by weight of the flour.

Such a dosage is particularly preferred when the masa and/or the masa foodstuff (preferably tortilla) is produced under alkaline conditions.

In one embodiment, when the xylanase is xylanase B, the xylanase dosage is the xylanase dosage is typically 200 to 3200 Units of xylanase activity (XU), preferably 400 to 1600 Units, per kg of corn-based flour and the hydrocolloid (preferably carboxymethylcellulose) is present in an amount of from 0.2 to 2%, preferably 0.4 to 1% by weight of the flour. Such a dosage is particularly preferred when the masa and/or the masa foodstuff (preferably tortilla) is produced under acidic conditions.

In one embodiment, when the xylanase is xylanase B, the xylanase dosage is typically 400 to 6400 Units of xylanase activity (XU), preferably 800 to 3200 Units, per kg of corn-based flour, and the hydrocolloid (preferably carboxymethylcellulose) is present in an amount of from 0.2 to 2%, preferably 0.4 to 1% by weight of the flour. Such a dosage is particularly preferred when the masa and/or the masa foodstuff (preferably tortilla) is produced under alkaline conditions.

### Masa Product

In one embodiment, the corn-based flour produced according to the process of the invention, and/or the corn flour, is mixed with water to produce a masa (also known as a masa dough).

The masa can be then be processed into various masa foodstuffs, examples of which include corn tortilla, soft tortilla, corn chips, tortilla chips, taco shells, corn flakes, tamales, derivatives and mixtures thereof. In one embodiment the masa foodstuff is a tortilla.

In one embodiment the corn is the sole cereal present in the masa product.

In another embodiment, the corn is present in the masa product as part of a mixture of cereals. In this embodiment, the corn may comprise at least 10% of the cereal mixture, such as at least 20% of the cereal mixture, such as at least 30% of the cereal mixture, such as at least 40% of the cereal mixture, such as at least 50% of the cereal mixture, such as at least 60% of the cereal mixture, such as at least 10% of the cereal mixture, such as at least 70% of the cereal mixture, such as at least 80% of the cereal mixture, such as at least 90% of the cereal mixture, such as at least 95% of the cereal mixture, such as such as at least 97% of the cereal mixture, such as at least 99% of the cereal mixture. In this embodiment, the other cereal may be any cereal typically used as a food. Examples of the other cereal include wheat, rye, barley and oats, especially wheat.

### Corn-Based Product

In some embodiments, the process of the present invention uses a xylanase enzyme (as defined herein) and a hydrocolloid. While this process may be used to form masa products as set out herein, such a process may also be used to produce corn-based products other than masa products.

The term “corn based product” as used herein means a plant composition which comprises (or consists essentially of or consists of) corn (maize) seed or grain or a by-product of corn grain.

In one embodiment the corn is the sole cereal present in the corn-based product.

In another embodiment, the corn is present in the masa product as part of a mixture of cereals. In this embodiment, the corn may comprise at least 10% of the cereal mixture, such as at least 20% of the cereal mixture, such as at least 30% of the cereal mixture, such as at least 40% of the cereal mixture, such as at least 50% of the cereal mixture, such as at least 60% of the cereal mixture, such as at least 10% of the cereal mixture, such as at least 70% of the cereal mixture, such as at least 80% of the cereal mixture, such as at least 90% of the cereal mixture, such as at least 95% of the cereal mixture, such as such as at least 97% of the cereal mixture, such as at least 99% of the cereal mixture. In this embodiment, the other cereal may be any cereal typically used as a food. Examples of the other cereal include wheat, rye, barley and oats, especially wheat.

### Other Ingredients

The xylanases A1, A2 and B and C (as defined herein) used in the present invention, such as may be used in combination with other components.

The combination of the present invention comprises the xylanases A1, A2 and B (as defined herein) used in the present invention and another component which is suitable for human or animal consumption and is capable of providing a medical or physiological benefit to the consumer.

Suitable additional enzymes for use in the present invention may be one or more of the enzymes selected from the group consisting of: endoglucanases (E.C. 3.2.1.4); celliobiohydrolases (E.C. 3.2.1.91), β-glucosidases (E.C. 3.2.1.21), cellulases (E.C. 3.2.1.74), lichenases (E.C. 3.1.1.73), lipases (E.C. 3.1.1.3), lipid acyltransferases (generally classified as E.C. 2.3.1.x), phospholipases (E.C. 3.1.1.4, E.C. 3.1.1.32 or E.C. 3.1.1.5), phytases (e.g. 6-phytase (E.C. 3.1.3.26) or a 3-phytase (E.C. 3.1.3.8), alpha-amylases (E.C. 3.2.1.1), other xylanases (E.C. 3.2.1.8, E.C. 3.2.1.32, E.C. 3.2.1.37, E.C. 3.1.1.72, E.C. 3.1.1.73), glucoamylases (E.C. 3.2.1.3), proteases (e.g. subtilisin (E.C. 3.4.21.62) or a bacillolysin (E.C. 3.4.24.28) or an alkaline serine protease (E.C. 3.4.21.x) or a keratinase (E.C. 3.4.x.x)) and/or mannanases (e.g. a β-mannanase (E.C. 3.2.1.78)).

In one embodiment the additional component may be a stabiliser or an emulsifier or a binder or carrier or an excipient or a diluent or a disintegrant.

The term “stabiliser” as used here is defined as an ingredient or combination of ingredients that keeps a product from changing over time.

The term “emulsifier” as used herein refers to an ingredient that prevents the separation of emulsions.

### Technical Effects and Surprising Findings

It has been surprisingly found by the present inventors that the incorporation of specific xylanase enzymes (as defined herein) into corn-based flour, such flour can be processed to produce a masa product (such as a tortilla) exhibiting improved characteristics such as texture, resistance, foldability and viscosity, compared with masa products prepared without such enzymes.

In particular, it has been found that when xylanase A1 (as defined above) is incorporated into a masa prepared under alkaline conditions, especially at a concentration of 0.08 to 1.28 mg/kg of corn-based flour preferably 0.16 to 0.64 mg/kg corn-based flour, this results in a masa with surprisingly improved viscosity compared with masa foodstuffs lacking this enzyme.

It has also been found that when xylanase A1 (as defined above) is incorporated into a masa prepared under acid conditions, especially at a concentration of 0.02 to 1.28 mg/kg of corn-based flour preferably 0.04 to 0.64 mg/kg corn-based flour, this results in a masa with surprisingly improved water holding capacity compared with masa foodstuffs lacking this enzyme.

It has also been found that when xylanase A1 (as defined above) is incorporated into a masa prepared under alkaline conditions, especially at a concentration of 0.08 to 1.28 mg/kg corn-based flour, preferably 0.16 to 0.64 mg/kg corn-based flour, together with carboxymethylcellulose in an amount of from 0.2 to 2%, preferably 0.4 to 1% by weight of the flour, this results in a masa with surprisingly improved viscosity compared with masa foodstuffs having the same concentration of CMC but lacking this enzyme.

It has also been found that when xylanase A1 (as defined above) is incorporated into a masa prepared under acid conditions, especially at a concentration of 0.02 to 1.28 mg/kg corn-based flour, preferably 0.04 to 0.64 mg/kg corn-based flour, together with carboxymethylcellulose in an amount of from 0.01 to 4%, preferably 0.4 to 1% by weight of the flour, this results in a masa with surprisingly improved water holding capacity compared with masa foodstuffs having the same concentration of CMC but lacking this enzyme.

It has also been found that when xylanase A1 (as defined above) is incorporated into a tortilla produced under alkaline conditions, especially at a concentration of 0.08 to 1.28 mg/kg of corn-based flour, preferably 0.16 to 0.64 mg/kg corn-based flour, together with carboxymethylcellulose in an amount of from 0.2 to 2%, preferably 0.4 to 1% by weight of the flour, this results in a tortilla with surprisingly improved flexibility and resistance compared with tortillas lacking this enzyme.

It has also been found that when xylanase B (as defined above) is incorporated into a masa prepared under alkaline conditions, especially at a concentration 0.08 to 1.2 mg/kg corn-based flour, preferably 0.15 to 0.6 mg/kg corn-based flour this results in a masa with surprisingly improved viscosity compared with masa lacking this enzyme.

It has also been found that when xylanase B (as defined above) is incorporated into a masa prepared under alkaline conditions, especially at a concentration of 0.08 to 1.2 mg/kg corn-based flour, preferably 0.15 to 0.6 mg/kg corn-based flour, together with carboxymethylcellulose in an amount of from 0.2 to 2%, preferably 0.4 to 1% by weight of the flour, this results in a masa with surprisingly improved viscosity compared with masa foodstuffs having the same concentration of CMC but lacking this enzyme.

It has also been found that when xylanase B (as defined above) is incorporated into a masa prepared under acid conditions, especially at a concentration of 0.04 to 1.2 mg/kg corn-based flour, preferably 0.15 to 0.6 mg/kg corn-based flour, together with carboxymethylcellulose in an amount of from 0.2 to 2%, preferably 0.4 to 1% by weight of the flour, this results in a masa product with surprisingly improved viscosity compared with masa foodstuffs having the same concentration of CMC but lacking this enzyme.

It has also been found that when xylanase B (as defined above) is incorporated into an alkaline tortilla, especially at a concentration 0.08 to 1.2 mg/kg corn-based flour, preferably 0.15 to 0.6 mg/kg corn-based flour, together with carboxymethylcellulose in an amount of from 0.2 to 2%, preferably 0.4 to 1% by weight of the flour, this results in a tortilla with surprisingly improved flexibility and resistance compared with masa foodstuffs (a) having the same concentration of CMC but lacking an enzyme or (b) having the same concentration of CMC but with an alpha-amylase enzyme instead of the xylanase B.

It has also been found that when xylanase A1 (as defined above) is incorporated into an alkaline tortilla, especially at a concentration of 0.04 to 0.64 mg/kg of corn-based flour, preferably 0.08 to 0.32 mg/kg of corn-based flour together with carboxymethylcellulose in an amount of from 0.2 to 2%, preferably 0.4 to 1% by weight of the flour, this results in a tortilla with surprisingly improved texture and resistance compared with tortillas having the same concentration of CMC but lacking this enzyme.

It has also been found that when xylanase B (as defined above) is incorporated into an alkaline tortilla, especially at a concentration of 0.08 to 1.2 mg/kg corn-based flour, preferably 0.15 to 0.6 mg/kg corn-based flour together with carboxymethylcellulose in an amount of from 0.2 to 2%, preferably 0.4 to 1% by weight of the flour, this results in a tortilla with surprisingly improved texture and resistance compared with tortillas having the same concentration of CMC but lacking this enzyme.

### Isolated

In one aspect, preferably the amino acid sequence, or nucleic acid, or enzyme according to the present invention is in an isolated form. The term “isolated” means that the sequence or enzyme or nucleic acid is at least substantially free from at least one other component with which the sequence, enzyme or nucleic acid is naturally associated in nature and as found in nature. The sequence, enzyme or nucleic acid of the present invention may be provided in a form that is substantially free of one or more contaminants with which the substance might otherwise be associated. Thus, for example it may be substantially free of one or more potentially contaminating polypeptides and/or nucleic acid molecules.

### Purified

In one aspect, preferably the sequence, enzyme or nucleic acid according to the present invention is in a purified form. The term “purified” means that the given component is present at a high level. The component is desirably the predominant component present in a composition. Preferably, it is present at a level of at least about 90%, or at least about 95% or at least about 98%, said level being determined on a dry weight/dry weight basis with respect to the total composition under consideration.

### Nucleotide Sequence

The scope of the present invention encompasses nucleotide sequences encoding proteins having the specific properties as defined herein.

The term “nucleotide sequence” as used herein refers to an oligonucleotide sequence or polynucleotide sequence, and variant, homologues, fragments and derivatives thereof (such as portions thereof). The nucleotide sequence may be of genomic or synthetic or recombinant origin, which may be double-stranded or single-stranded whether representing the sense or anti-sense strand.

The term “nucleotide sequence” in relation to the present invention includes genomic DNA, cDNA, synthetic DNA, and RNA. Preferably it means DNA, more preferably cDNA sequence coding for the present invention.

In a preferred embodiment, the nucleotide sequence when relating to and when encompassed by the per se scope of the present invention does not include the native nucleotide sequence according to the present invention when in its natural environment and when it is linked to its naturally associated sequence(s) that is/are also in its/their natural environment. For ease of reference, we shall call this preferred embodiment the “non-native nucleotide sequence”. In this regard, the term “native nucleotide sequence” means an entire nucleotide sequence that is in its native environment and when operatively linked to an entire promoter with which it is naturally associated, which promoter is also in its native environment. However, the amino acid sequence encompassed by scope the present invention can be isolated and/or purified post expression of a nucleotide sequence in its native organism. Preferably, however, the amino acid sequence encompassed by scope of the present invention may be expressed by a nucleotide sequence in its native organism but wherein the nucleotide sequence is not under the control of the promoter with which it is naturally associated within that organism.

Typically, the nucleotide sequence encompassed by the scope of the present invention is prepared using recombinant DNA techniques (i.e. recombinant DNA). However, in an alternative embodiment of the invention, the nucleotide sequence could be synthesised, in whole or in part, using chemical methods well known in the art (see Caruthers M H et al., (1980) Nuc Acids Res Symp Ser 215-23 and Horn T et al., (1980) Nuc Acids Res Symp Ser 225-232).

### Preparation of the Nucleotide Sequence

A nucleotide sequence encoding either a protein which has the specific properties as defined herein or a protein which is suitable for modification may be identified and/or isolated and/or purified from any cell or organism producing said protein. Various methods are well known within the art for the identification and/or isolation and/or purification of nucleotide sequences. By way of example, PCR amplification techniques to prepare more of a sequence may be used once a suitable sequence has been identified and/or isolated and/or purified.

By way of further example, a genomic DNA and/or cDNA library may be constructed using chromosomal DNA or messenger RNA from the organism producing the enzyme. If the amino acid sequence of the enzyme is known, labelled oligonucleotide probes may be synthesised and used to identify enzyme-encoding clones from the genomic library prepared from the organism. Alternatively, a labelled oligonucleotide probe containing sequences homologous to another known enzyme gene could be used to identify enzyme-encoding clones. In the latter case, hybridisation and washing conditions of lower stringency are used.

Alternatively, enzyme-encoding clones could be identified by inserting fragments of genomic DNA into an expression vector, such as a plasmid, transforming enzyme-negative bacteria with the resulting genomic DNA library, and then plating the transformed bacteria onto agar plates containing a substrate for enzyme (i.e. maltose), thereby allowing clones expressing the enzyme to be identified.

In a yet further alternative, the nucleotide sequence encoding the enzyme may be prepared synthetically by established standard methods, e.g. the phosphoroamidite method described by Beucage S. L. et al., (1981) Tetrahedron Letters 22, p 1859-1869, or the method described by Matthes et al., (1984) EMBO J. 3, p 801-805. In the phosphoroamidite method, oligonucleotides are synthesised, e.g. in an automatic DNA synthesiser, purified, annealed, ligated and cloned in appropriate vectors.

The nucleotide sequence may be of mixed genomic and synthetic origin, mixed synthetic and cDNA origin, or mixed genomic and cDNA origin, prepared by ligating fragments of synthetic, genomic or cDNA origin (as appropriate) in accordance with standard techniques. Each ligated fragment corresponds to various parts of the entire nucleotide sequence. The DNA sequence may also be prepared by polymerase chain reaction (PCR) using specific primers, for instance as described in U.S. Pat. No. 4,683,202 or in Saiki R K et al., (Science (1988) 239, pp 487-491).

### Hybridisation

The present invention also encompasses sequences that are complementary to the nucleic acid sequences of the present invention or sequences that are capable of hybridising either to the sequences of the present invention or to sequences that are complementary thereto.

The term “hybridisation” as used herein shall include “the process by which a strand of nucleic acid joins with a complementary strand through base pairing” as well as the process of amplification as carried out in polymerase chain reaction (PCR) technologies.

The present invention also encompasses the use of nucleotide sequences that are capable of hybridising to the sequences that are complementary to the sequences presented herein, or any fragment or derivative thereof.

The term “variant” also encompasses sequences that are complementary to sequences that are capable of hybridising to the nucleotide sequences presented herein.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to the nucleotide sequences presented herein.

More preferably, the term “variant” encompasses sequences that are complementary to sequences that are capable of hybridising under high stringency conditions (e.g. 65° C. and 0.1×SSC {1×SSC=0.15 M NaCl, 0.015 M Na₃citrate pH 7.0}) to the nucleotide sequences presented herein.

The present invention also relates to nucleotide sequences that can hybridise to the nucleotide sequences of the present invention (including complementary sequences of those presented herein).

The present invention also relates to nucleotide sequences that are complementary to sequences that can hybridise to the nucleotide sequences of the present invention (including complementary sequences of those presented herein).

Preferably hybridisation is analysed over the whole of the sequences taught herein.

### Amino Acid Sequences

The scope of the present invention also encompasses amino acid sequences of enzymes having the specific properties as defined herein.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”.

The amino acid sequence may be prepared/isolated from a suitable source, or it may be made synthetically or it may be prepared by use of recombinant DNA techniques.

Preferably the amino acid sequence when relating to and when encompassed by the per se scope of the present invention is not a native enzyme. In this regard, the term “native enzyme” means an entire enzyme that is in its native environment and when it has been expressed by its native nucleotide sequence.

### Sequence Identity or Sequence Homology

The present invention also encompasses the use of sequences having a degree of sequence identity or sequence homology with amino acid sequence(s) of a polypeptide having the specific properties defined herein or of any nucleotide sequence encoding such a polypeptide (hereinafter referred to as a “homologous sequence(s)”). Here, the term “homologue” means an entity having a certain homology with the subject amino acid sequences and the subject nucleotide sequences. Here, the term “homology” can be equated with “identity”.

The homologous amino acid sequence and/or nucleotide sequence should provide and/or encode a polypeptide which retains the functional activity and/or enhances the activity of the enzyme.

In the present context, in some embodiments a homologous sequence is taken to include an amino acid or a nucleotide sequence which may be at least 97.7% identical, preferably at least 98 or 99% identical to the subject sequence.

In some embodiments a homologous sequence is taken to include an amino acid or a nucleotide sequence which may be at least 85% identical, preferably at least 90 or 95% identical to the subject sequence.

Typically, the homologues will comprise the same active sites etc. as the subject amino acid sequence for instance. Although homology can also be considered in terms of similarity (i.e. amino acid residues having similar chemical properties/functions), in the context of the present invention it is preferred to express homology in terms of sequence identity.

In one embodiment, a homologous sequence is taken to include an amino acid sequence or nucleotide sequence which has one or several additions, deletions and/or substitutions compared with the subject sequence.

In the present context, “the subject sequence” relates to the nucleotide sequence or polypeptide/amino acid sequence according to the invention.

A “parent nucleic acid” or “parent amino acid” means a nucleic acid sequence or amino acid sequence, encoding or coding for the parent polypeptide, respectively.

In one embodiment the present invention relates to a protein whose amino acid sequence is represented herein or a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

Suitably, the degree of identity with regard to an amino acid sequence is determined over at least 20 contiguous amino acids, preferably over at least 30 contiguous amino acids, preferably over at least 40 contiguous amino acids, preferably over at least 50 contiguous amino acids, preferably over at least 60 contiguous amino acids, preferably over at least 100 contiguous amino acids, preferably over at least 200 contiguous amino acids.

In one embodiment the present invention relates to a nucleic acid sequence (or gene) encoding a protein whose amino acid sequence is represented herein or encoding a protein derived from this (parent) protein by substitution, deletion or addition of one or several amino acids, such as 2, 3, 4, 5, 6, 7, 8, 9 amino acids, or more amino acids, such as 10 or more than 10 amino acids in the amino acid sequence of the parent protein and having the activity of the parent protein.

In the present context, in one embodiment a homologous sequence or foreign sequence is taken to include a nucleotide sequence which may be at least 97.7% identical, preferably at least 98 or 99% identical to a nucleotide sequence encoding a polypeptide of the present invention (the subject sequence).

In another embodiment, a homologous sequence is taken to include a nucleotide sequence which may be at least 85% identical, preferably at least 90 or 95% identical to a nucleotide sequence encoding a polypeptide of the present invention (the subject sequence).

Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology or % identity between two or more sequences.

% homology or % identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology or % identity when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons.

Calculation of maximum % homology or % identity therefore firstly requires the production of an optimal alignment, taking into consideration gap penalties. A suitable computer program for carrying out such an alignment is the Vector NTI (Invitrogen Corp.). Examples of software that can perform sequence comparisons include, but are not limited to, the BLAST package (see Ausubel et al 1999 Short Protocols in Molecular Biology, 4th Ed—Chapter 18), BLAST 2 (see FEMS Microbiol Lett 1999 174(2): 247-50; FEMS Microbiol Lett 1999 177(1): 187-8 and tatiana@ncbi.nlm.nih.gov), FASTA (Altschul et al 1990 J. Mol. Biol. 403-410) and AlignX for example. At least BLAST, BLAST 2 and FASTA are available for offline and online searching (see Ausubel et al 1999, pages 7-58 to 7-60), such as for example in the GenomeQuest search tool (www.genomequest.com).

Although the final % homology or % identity can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. Vector NTI programs generally use either the public default values or a custom symbol comparison table if supplied (see user manual for further details). For some applications, it is preferred to use the default values for the Vector NTI package.

Alternatively, percentage homologies may be calculated using the multiple alignment feature in Vector NTI (Invitrogen Corp.), based on an algorithm, analogous to CLUSTAL (Higgins D G & Sharp P M (1988), Gene 73(1), 237-244).

Once the software has produced an optimal alignment, it is possible to calculate homology, preferably % sequence identity. The software typically does this as part of the sequence comparison and generates a numerical result.

Should Gap Penalties be used when determining sequence identity, then preferably the following parameters are used for pairwise alignment:

| FOR BLAST | |
|---|---|
| GAP OPEN | 9 |
| GAP EXTENSION | 2 |

| FOR CLUSTAL | DNA | PROTEIN |
|---|---|---|
| Weight Matrix | IUB | Gonnet 250 |
| GAP OPENING | 15 | 10 |
| GAP EXTEND | 6.66 | 0.1 |

In one embodiment, CLUSTAL may be used with the gap penalty and gap extension set as defined above.

Suitably, the degree of identity with regard to a nucleotide sequence or protein sequence is determined over at least 20 contiguous nucleotides/amino acids, preferably over at least 30 contiguous nucleotides/amino acids, preferably over at least 40 contiguous nucleotides/amino acids, preferably over at least 50 contiguous nucleotides/amino acids, preferably over at least 60 contiguous nucleotides/amino acids, preferably over at least 100 contiguous nucleotides/amino acids.

Suitably, the degree of identity with regard to a nucleotide sequence sequence is determined over at least 100 contiguous nucleotides, preferably over at least 200 contiguous nucleotides, preferably over at least 300 contiguous nucleotides, preferably over at least 400 contiguous nucleotides, preferably over at least 500 contiguous nucleotides, preferably over at least 600 contiguous nucleotides, preferably over at least 700 contiguous nucleotides, preferably over at least 800 contiguous nucleotides.

Suitably, the degree of identity with regard to a nucleotide sequence may be determined over the whole sequence taught herein.

Suitably, the degree of identity with regard to a protein (amino acid) sequence is determined over at least 100 contiguous amino acids, preferably over at least 200 contiguous amino acids, preferably over at least 300 contiguous amino acids.

Suitably, the degree of identity with regard to an amino acid or protein sequence may be determined over the whole sequence taught herein.

In the present context, the term “query sequence” means a homologous sequence or a foreign sequence, which is aligned with a subject sequence in order to see if it falls within the scope of the present invention. Accordingly, such query sequence can for example be a prior art sequence or a third party sequence.

In one preferred embodiment, the sequences are aligned by a global alignment program and the sequence identity is calculated by identifying the number of exact matches identified by the program divided by the length of the subject sequence.

In one embodiment, the degree of sequence identity between a query sequence and a subject sequence is determined by 1) aligning the two sequences by any suitable alignment program using the default scoring matrix and default gap penalty, 2) identifying the number of exact matches, where an exact match is where the alignment program has identified an identical amino acid or nucleotide in the two aligned sequences on a given position in the alignment and 3) dividing the number of exact matches with the length of the subject sequence.

In yet a further preferred embodiment, the global alignment program is selected from the group consisting of CLUSTAL and BLAST (preferably BLAST) and the sequence identity is calculated by identifying the number of exact matches identified by the program divided by the length of the subject sequence.

The sequences may also have deletions, insertions or substitutions of amino acid residues result in a functionally equivalent substance. Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The present invention also encompasses homologous substitution (substitution and replacement are both used herein to mean the interchange of an existing amino acid residue, with an alternative residue) that may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as O), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-l-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

Suitably there may be at least 2 conservative substitutions, such as at least 3 or at least 4 or at least 5.

Suitably there may be less than 15 conservative substitutions, such as less than 12, less than 10, or less than 8 or less than 5.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in order to enhance the in vivo activity or life span of nucleotide sequences of the present invention.

The present invention also encompasses the use of nucleotide sequences that are complementary to the sequences presented herein, or any derivative, fragment or derivative thereof. If the sequence is complementary to a fragment thereof then that sequence can be used as a probe to identify similar coding sequences in other organisms etc.

Polynucleotides which are not 100% homologous to the sequences of the present invention but fall within the scope of the invention can be obtained in a number of ways. Other variants of the sequences described herein may be obtained for example by probing DNA libraries made from a range of individuals, for example individuals from different populations. In addition, other homologues may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of any one of the sequences in the attached sequence listings under conditions of medium to high stringency. Similar considerations apply to obtaining species homologues and allelic variants of the polypeptide or nucleotide sequences of the invention.

Variants and strain/species homologues may also be obtained using degenerate PCR which will use primers designed to target sequences within the variants and homologues encoding conserved amino acid sequences within the sequences of the present invention. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences.

Alternatively, such polynucleotides may be obtained by site directed mutagenesis of characterised sequences. This may be useful where for example silent codon sequence changes are required to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

Polynucleotides (nucleotide sequences) of the invention may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 15, preferably at least 20, for example at least 25, 30 or 40 nucleotides in length, and are also encompassed by the term polynucleotides of the invention as used herein.

Polynucleotides such as DNA polynucleotides and probes according to the invention may be produced recombinantly, synthetically, or by any means available to those of skill in the art. They may also be cloned by standard techniques.

In general, primers will be produced by synthetic means, involving a stepwise manufacture of the desired nucleic acid sequence one nucleotide at a time. Techniques for accomplishing this using automated techniques are readily available in the art.

Longer polynucleotides will generally be produced using recombinant means, for example using a PCR (polymerase chain reaction) cloning techniques. The primers may be designed to contain suitable restriction enzyme recognition sites so that the amplified DNA can be cloned into a suitable cloning vector.

### Amino Acid Numbering

In the present invention, a specific numbering of amino acid residue positions in the xylanases used in the present invention may be employed. By alignment of the amino acid sequence of a sample xylanases with the xylanase of the present invention (particularly SEQ ID No. 3) it is possible to allot a number to an amino acid residue position in said sample xylanase which corresponds with the amino acid residue position or numbering of the amino acid sequence shown in SEQ ID NO:3 of the present invention.

### Host Cells

The term “host cell”—in relation to the present invention includes any cell that comprises either the nucleotide sequence or an expression vector as described above and which is used in the recombinant production of a protein having the specific properties as defined herein.

In one embodiment the organism is an expression host.

Thus, a further embodiment of the present invention provides host cells transformed or transfected with a nucleotide sequence that expresses the protein of the present invention. The cells will be chosen to be compatible with the said vector and may for example be prokaryotic (for example bacterial), fungal or yeast cells.

Examples of suitable bacterial host organisms are gram positive or gram negative bacterial species.

In one embodiment the xylanases taught herein are expressed in the expression host Trichoderma reesei.

In some embodiments the expression host for the xylanases taught herein may be one or more of the following fungal expression hosts: Fusarium spp. (such as Fusarium oxysporum); Aspergillus spp. (such as Aspergillus niger, A. oryzae, A. nidulans, or A. awamori) or Trichoderma spp. (such as T. reesei).

In some embodiments the expression host may be one or more of the following bacterial expression hosts: Streptomyces spp. or Bacillus spp. (e.g. Bacillus subtilis or B. licheniformis).

The use of suitable host cells—such as yeast and fungal host cells—may provide for post-translational modifications (e.g. myristoylation, glycosylation, truncation, lipidation and tyrosine, serine or threonine phosphorylation) as may be needed to confer optimal biological activity on recombinant expression products of the present invention.

### Organism

The term “organism” in relation to the present invention includes any organism that could comprise the nucleotide sequence coding for the polypeptide according to the present invention and/or products obtained therefrom, and/or wherein a promoter can allow expression of the nucleotide sequence according to the present invention when present in the organism.

In one embodiment the organism is an expression host.

Suitable organisms may include a prokaryote, fungus, yeast or a plant.

The term “transgenic organism” in relation to the present invention includes any organism that comprises the nucleotide sequence coding for the polypeptide according to the present invention and/or the products obtained therefrom, and/or wherein a promoter can allow expression of the nucleotide sequence according to the present invention within the organism. Preferably the nucleotide sequence is incorporated in the genome of the organism.

The term “transgenic organism” does not cover native nucleotide coding sequences in their natural environment when they are under the control of their native promoter which is also in its natural environment.

Therefore, the transgenic organism of the present invention includes an organism comprising any one of, or combinations of, the nucleotide sequence coding for the polypeptide according to the present invention, constructs according to the present invention, vectors according to the present invention, plasmids according to the present invention, cells according to the present invention, tissues according to the present invention, or the products thereof.

For example the transgenic organism may also comprise the nucleotide sequence coding for the polypeptide of the present invention under the control of a heterologous promoter.

### Transformation of Host Cells/Organism

As indicated earlier, the host organism can be a prokaryotic or a eukaryotic organism. Examples of suitable prokaryotic hosts include E. coli, Streptomyces spp. and Bacillus spp., e.g. Bacillus subtilis.

Teachings on the transformation of prokaryotic hosts is well documented in the art, for example see Sambrook et al (Molecular Cloning: A Laboratory Manual, 2nd edition, 1989, Cold Spring Harbor Laboratory Press). If a prokaryotic host is used then the nucleotide sequence may need to be suitably modified before transformation—such as by removal of introns.

Filamentous fungi cells may be transformed using various methods known in the art—such as a process involving protoplast formation and transformation of the protoplasts followed by regeneration of the cell wall in a manner known. The use of Aspergillus as a host microorganism is described in EP 0 238 023.

Transformation of prokaryotes, fungi and yeasts are generally well known to one skilled in the art.

A host organism may be a fungus—such as a mould. Examples of suitable such hosts include any member belonging to the genera Trichoderma (e.g. T. reesei), Thermomyces, Acremonium, Fusarium, Aspergillus, Penicillium, Mucor, Neurospora and the like.

In one embodiment, the host organism may be a fungus. In one preferred embodiment the host organism belongs to the genus Trichoderma, e.g. T. reesei).

### Culturing and Production

Host cells transformed with the nucleotide sequence for use in the present invention may be cultured under conditions conducive to the production of the encoded polypeptide and which facilitate recovery of the polypeptide from the cells and/or culture medium.

The medium used to cultivate the cells may be any conventional medium suitable for growing the host cell in questions and obtaining expression of the polypeptide.

The protein produced by a recombinant cell may be displayed on the surface of the cell.

The protein may be secreted from the host cells and may conveniently be recovered from the culture medium using well-known procedures.

### Secretion

Often, it is desirable for the protein to be secreted from the expression host into the culture medium from where the protein may be more easily recovered. According to the present invention, the secretion leader sequence may be selected on the basis of the desired expression host. Hybrid signal sequences may also be used with the context of the present invention.

### General Recombinant DNA Methodology Techniques

The present invention employs, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

### EXAMPLES

### Example 1

### Cloning of Fusarium verticillioides Xylanase (FveXyn4) (Xylanase A1)

Genomic DNA isolated from a strain of Fusarium verticillioides was used for amplifying a xylanase gene. The sequence of the cloned gene, called the FveXyn4 gene, is depicted in SEQ ID No. 4. The protein encoded by the FveXyn4 gene is depicted in SEQ ID No. 1. The protein product of gene FveXyn4 belongs to glycosyl hydrolase family 10 (GH10) based on the PFAM search (http://pfam.sanger.ac.uk/). At the N-terminus, FveXyn4 protein has a 15 amino acid signal peptide predicted by SignalP-NN (Emanuelsson et al., Nature Protocols, 2:953-971, 2007). This indicates that FveXyn4 is a secreted glycosyl hydrolase.

### Example 2

### Expression of FveXyn4 Protein (Xylanase A1)

The FveXyn4 gene was amplified from genomic DNA of Fusarium verticillioides using the following primers: Primer 1 5′-caccATGAAGCTGTCTTCTTTCCTCTA-3′ (SEQ ID No. 23), and Primer 2 5′-TTTTTAGCGGAGAGCGTTGACAACAGC-3′ (SEQ ID No. 24). The PCR product was cloned into pENTR/D-TOPO vector (Invitrogen K2400) to generate the FveXyn4 pEntry plasmid. The expression plasmid pZZH254 was obtained by Gateway cloning reaction between the FveXyn4 pEntry plasmid and pTrex3gM expression vector (described in US 2011/0136197 A1) using Gateway® LR Clonase® II enzyme kit (Invitrogen 11791). A map of plasmid pZZH254 is provided as FIG. 10A. The sequence of the FveXyn4 gene was confirmed by DNA sequencing (SEQ ID No. 4). The plasmid pZZH254 was transformed into a quad deleted Trichoderma reesei strain (described in WO 05/001036) using biolistic method (Te'o VS et al., J Microbiol Methods, 51:393-9, 2002).

Following sequence confirmation, protoplasts of a quad deleted T. reesei strain (described in WO 05/001036) were transformed with the expression plasmid pZZH254 using the PEG protoplast method (Penttila et al, Gene, 61:155-164, 1987). For protoplast preparation, spores were grown for about 10 hours at 24° C. in Trichoderma Minimal Medium MM (20 g/L glucose, 15 g/L KH₂PO₄, pH 4.5, 5 g/L (NH₄)2SO₄, 0.6 g/L MgSO₄×7H₂O, 0.6 g/L CaCl₂×2H₂O, 1 ml of 1000× T. reesei Trace elements solution (175 g/L Citric Acid anhydrous, 200 g/L FeSO₄×7H₂O, 16 g/L ZnSO₄×7H₂O, 3.2 g/L CuSO₄, 1.4 g/L MnSO₄×H₂O, and 0.8 g/L Boric Acid). Germinating spores were harvested by centrifugation and treated with 30 mg/mL Vinoflow FCE (Novozymes, AG Switzerland) solution for from 7 hours to overnight at 30° C. at 100 rpm to lyse the fungal cell walls. Protoplasts were washed in 0.1 M Tris HCl buffer (pH 7) containing 0.6 M sorbitol and resuspended in 10 mM Tris HCl buffer (pH 7.5) containing 1.2 M sorbitol and 10 mM calcium chloride. For PEG transformation, approximately 1 μg of DNA and 1-5×10⁷ protoplasts in a total volume of 200 μl were treated with 2 ml of 25% PEG solution, diluted with 2 volumes of 1.2 M sorbitol/10 mM Tris, pH 7.5/10 mM CaCl₂ solution. Transformants were selected on a medium containing acetamide as a sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies (about 50-100) appeared in about 1 week. After growth on acetamide plates, the spores were collected and reselected on acetamide plates. After 5 days, the spores were collected using 10% glycerol, and 1×10⁸ spores were inoculated in a 250 ml shake flask with 30 ml Glucose/Sophorose defined medium for protein expression. Protein expression was confirmed by SDS-PAGE. The spore suspension was subsequently grown in a 7 L fermentor in a defined medium containing 60% glucose-sophorose feed. Glucose/Sophorose defined medium (per liter) consists of (NH₄)₂SO₄ 5 g, PIPPS buffer 33 g, Casamino Acids 9 g, KH₂PO₄ 4.5 g, CaCl₂ (anhydrous) 1 g, MgSO₄.7H₂O 1 g, pH to 5.5 adjusted with 50% NaOH with Milli-Q H₂O to bring to 966.5 mL. After sterilization, the following were added: 26 mL 60% Glucose/Sophrose, and 400× T. reesei Trace Metals 2.5 mL.

FveXyn4 was purified from concentrated fermentation broth of a 7 L fermentor culture using two chromatography columns. Concentrated fermentation broth buffered in 20 mM sodium phosphate buffer pH 6.0 containing 1 M ammonium sulfate was loaded on a hydrophobic interaction chromatography column (Sepharose Phenyl FF, 26/10). The protein was eluted from the column using a linear gradient of equilibration/wash buffer to 20 mM sodium phosphate buffer pH 6.0. The fraction containing FveXyn4 protein was loaded onto a gel filtration column (HiLoad Superdex 75 μg 26/60), and the mobile phase used was 20 mM sodium phosphate, pH 7.0, containing 0.15 M NaCl. The purified protein was concentrated using a 3K Amicon Ultra-15 device and the concentrated protein fraction was processed into a powder (as described used in further studies.

The nucleotide sequence of FveXyn4 gene from expression plasmid pZZH254 is set forth as SEQ ID No. 4. The signal sequence is shown in bold (upper case), and the predicted intron is shown in bold and lowercase.

The amino acid sequence of FveXyn4 protein expressed from plasmid pZZH254 is set forth as SEQ ID No. 1. The signal sequence predicted by SignalP-NN software is shown underlined. This is the pre-pro-protein.

The amino acid sequence of the predicted mature form of FveXyn4 protein is set forth as SEQ ID No. 3. This is the active form of the enzyme. SEQ ID No. 2 shows the pro-protein, i.e. before post-translational modification. Depending on the host the post-translation modification may vary and therefore the present invention also encompasses mature, active forms of SEQ ID No. 2.

### Example 3

### Xylanase Activity of FveXyn4 (Xylanase A1)

FveXyn4 belongs to the glycosyl hydrolase 10 family (GH10, CAZy number). The beta 1-4 xylanase activity of FveXyn4 was measured using 1% xylan from birch wood (Sigma 95588) or 1% arabinoxylan from wheat flour (Megazyme P-WAXYM) as substrates. The assay was performed in 50 mM sodium citrate pH 5.3, 0.005% Tween-80 buffer at 50° C. for 10 minutes.

The released reducing sugar was quantified by reaction with 3, 5-Dinitrosalicylic acid and measurement of absorbance at 540 nm. The enzyme activity is quantified relative to a xylose standard curve. In this assay, one xylanase unit (U) is defined as the amount of enzyme required to generate 1 micromole of xylose reducing sugar equivalents per minute under the conditions of the assay.

### Example 4

### Cloning of Fusarium oxysporum Xylanase FoxXyn2 (Xylanase A2)

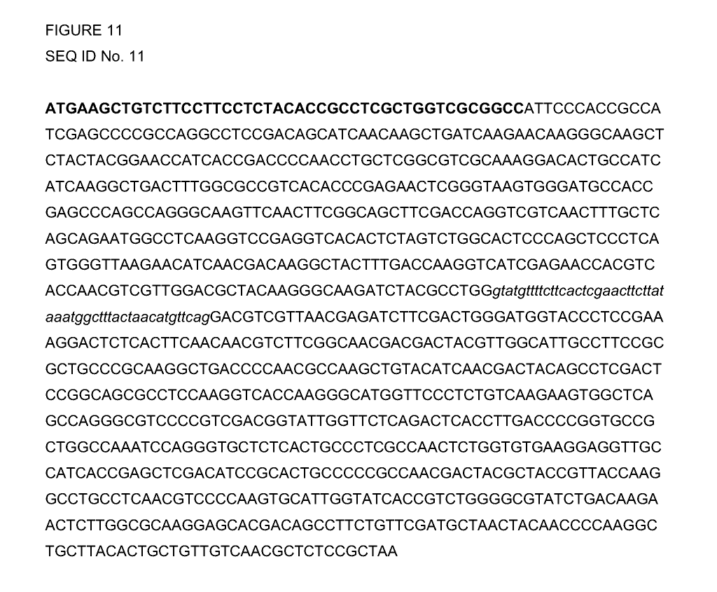

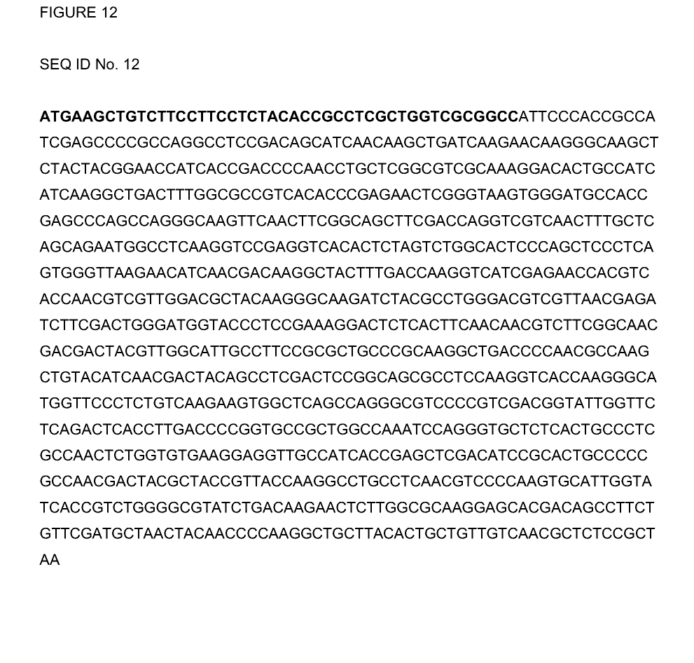

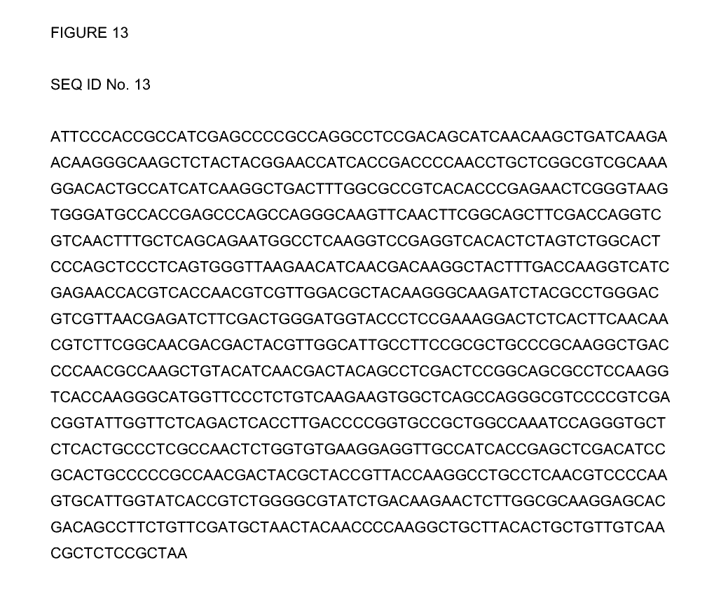

The nucleotide sequence of the FoxXyn2 gene isolated from Fusarium oxysporum is set forth as SEQ ID Nos 11, 12 and 13. The predicted intron is shown in SEQ ID No. 11 (FIG. 11) in italics and lowercase.

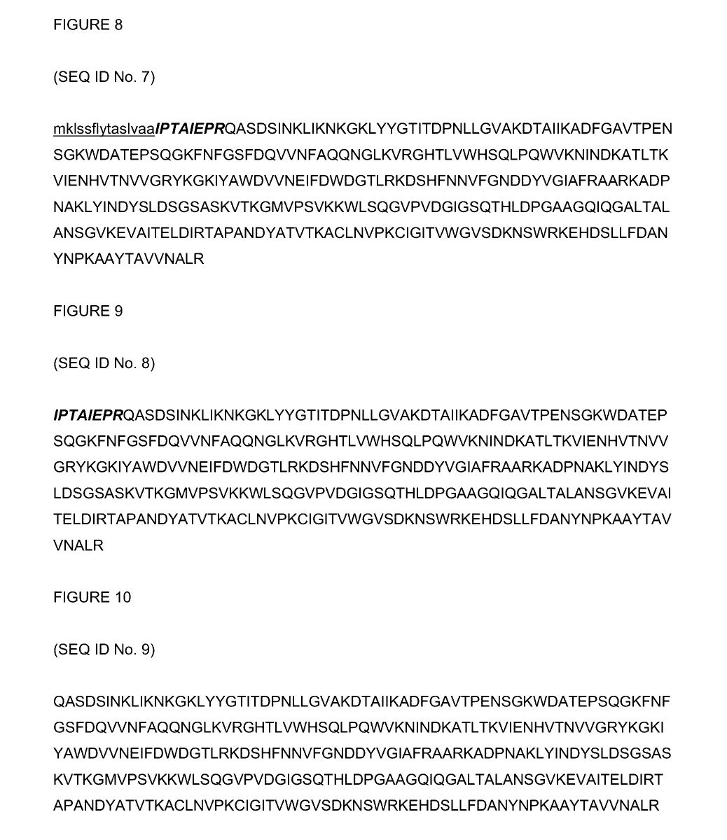

The amino acid sequence of the FoxXyn2 precursor protein is set forth as SEQ ID No. 7 (FIG. 8). The predicted signal sequence is shown in italics and lowercase.

The amino acid sequence of the predicted mature forms of FoxXyn2 is set forth as SEQ ID No. 7 and 8 (shown in FIGS. 8A and 10). SEQ ID No. 8 shows a section of the polypeptide that may be cleaved before full maturation of the protein. The active form of the protein may be with or without this section, and thus the active protein may have SEQ ID No. 8 or SEQ ID No. 9.

The protein product of gene FoxXyn2 belongs to glycosyl hydrolase family 10. This suggests that FoxXyn2 is a secreted glycosyl hydrolase.

### Example 5

### Expression of FoxXyn2 Protein (Xylanase A2)

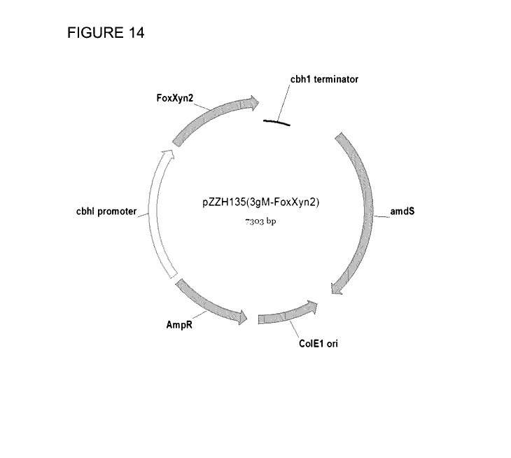

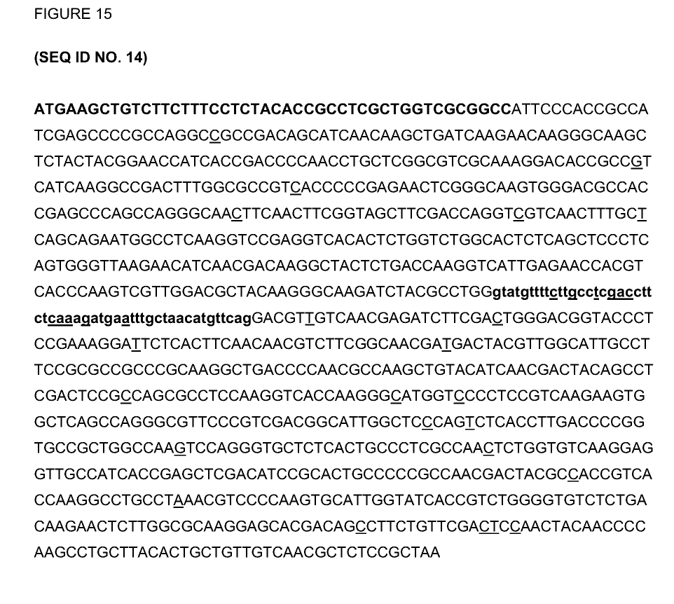

The FoxXyn2 gene was amplified from genomic DNA of Fusarium oxysporum using the following primers: Primer 1 5′-ccgcggccgcaccATGAAGCTGTCTTCCTTCCTCTACACC-3′ (SEQ ID NO: 25), and Primer 2 5′-ccggcgcgcccttaTTAGCGGAGAGCGTTGACAACAG-3′ (SEQ ID NO: 26). After digested with Not I and Asc I, the PCR product was cloned into pTrex3gM expression vector (described in US 2011/0136197 A1) digested with the same restriction enzymes, and the resulting plasmid was labeled pZZH135. A plasmid map of pZZH135 is provided in FIG. 14. The sequence of the FoxXyn2 gene was confirmed by DNA sequencing.

The plasmid pZZH135 was transformed into a quad deleted Trichoderma reesei strain (described in WO 05/001036, incorporated herein by reference) using biolistic method (taught in Te'o VS et al., J Microbiol Methods, 51:393-9, 2002). The protein isolated from the culture supernatant after filtration was used to perform SDS-PAGE analysis and xylanase activity assay to confirm enzyme expression.

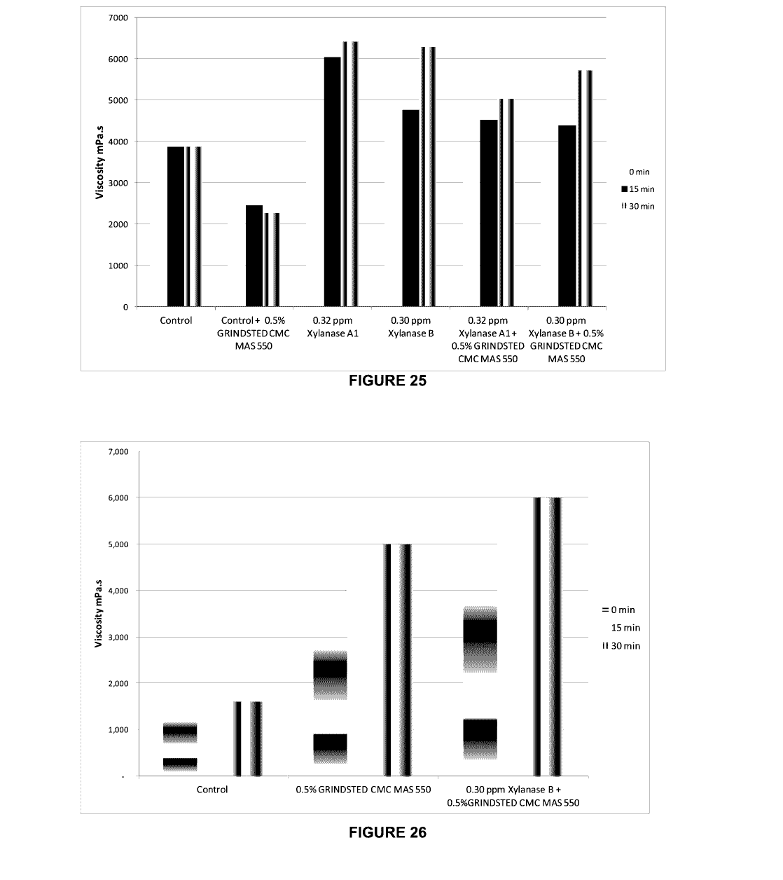

The nucleotide sequence of FoxXyn2 gene from expression plasmid pZZH135 is set forth as SEQ ID No. 11 (FIG. 25). The signal sequence is shown in bold, and the predicted intron is shown in italics and lowercase.

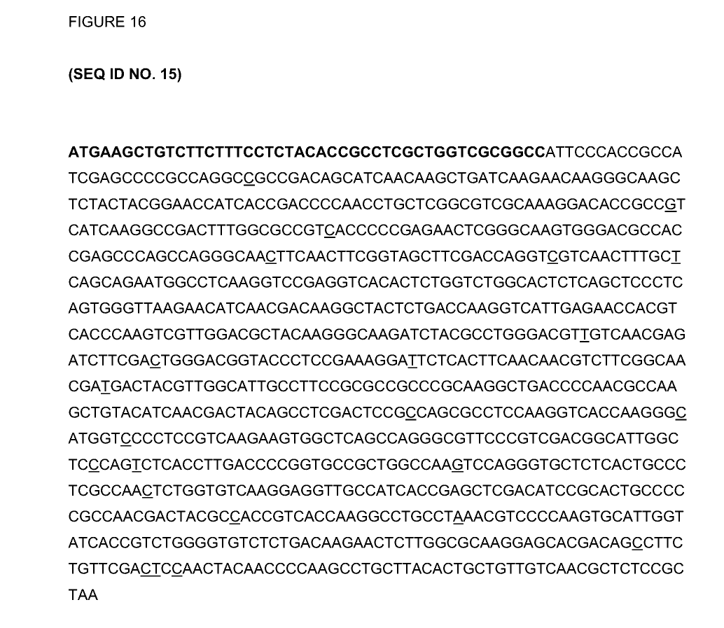

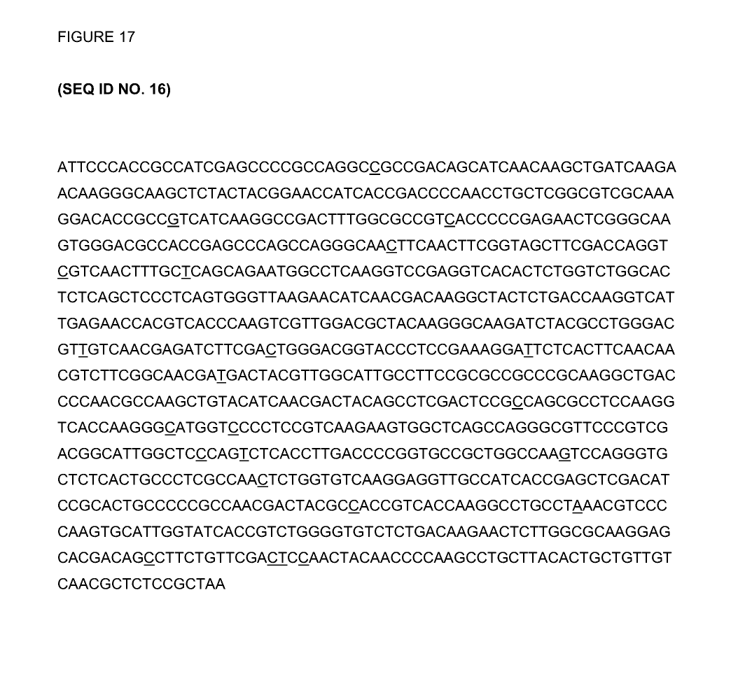

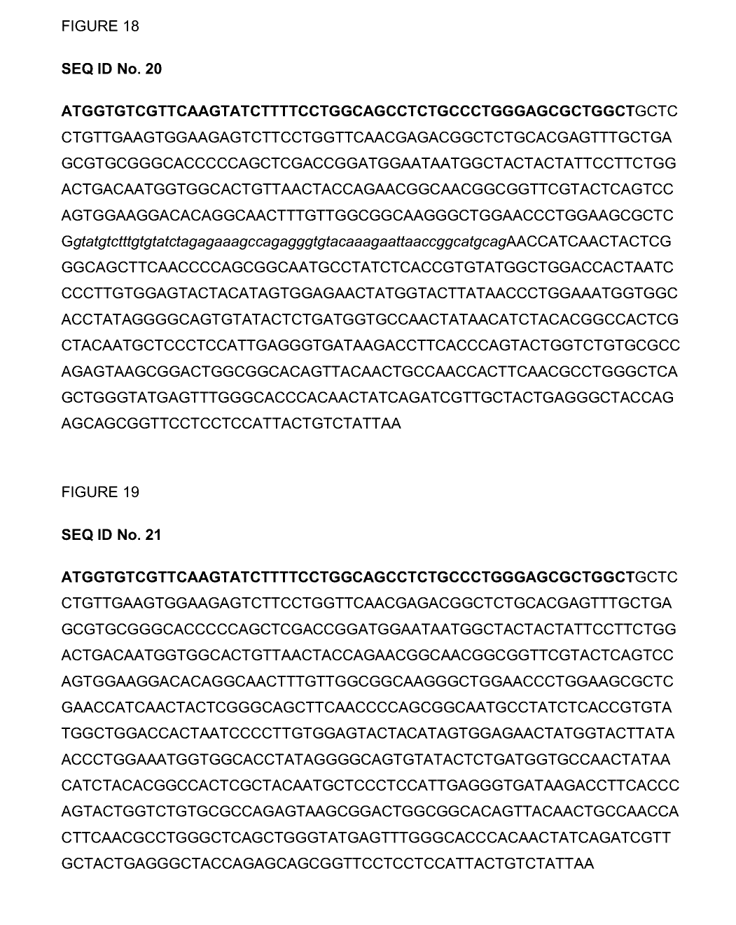

The amino acid sequence of FoxXyn2 protein expressed from plasmid pZZH135 is set forth as SEQ ID No. 7 (FIG. 10). The signal sequence is shown in italics. The amino acid sequence of the mature form of FoxXyn2 protein is set forth as SEQ ID No. 8 (FIG. 16).

FoxXyn2 protein was purified from culture supernatant using affinity chromatography resin Blue Sepharose, 6FF, and samples were used for biochemical characterization as described in subsequent examples.

### Example 6

### Xylanase Activity of FoxXyn2 (Xylanase A2)

FoxXyn2 belongs to the glycosyl hydrolase 10 family (GH10, CAZy number). The beta 1-4 xylanase activity of FoxXyn2 was measured using 1% xylan from birch wood (Sigma 95588) or 1% arabinoxylan from wheat flour (Megazyme P-WAXYM) as substrates. The assay was performed in 50 mM sodium citrate pH 5.3, 0.005% Tween-80 buffer at 50° C. for 10 minutes.

The released reducing sugar was quantified by reaction with 3, 5-Dinitrosalicylic acid and measurement of absorbance at 540 nm. The enzyme activity is quantified relative to a xylose standard curve. In this assay, one xylanase unit (U) is defined as the amount of enzyme required to generate 1 micromole of xylose reducing sugar equivalents per minute under the conditions of the assay.

### Example 7

### Cloning of the Aspergillus clavatus Xylanase AclXyn5 (Xylanase B)

The entire genomic sequence data of Aspergillus clavatus is available online (http://www.broadinstitute.orq/annotation/qenome/asperqillus qroup/GeneDetails.html?sp=S7000001156845959) One of the genes (ACLA_063140) identified in Aspergillus clavatus encodes a glycosyl hydrolase with homology to xylanases of various other fungi as determined from a BLAST search (Altschul et al., J Mol Biol, 215: 403-410, 1990). The nucleotide sequence of this gene, called AclXyn5 gene, is depicted as SEQ ID NO.20. The protein encoded by the AclXyn5 gene is depicted as SEQ ID NO. 17, and has received the accession number A1CCU0 in Uniprot database. Genomic DNA of Aspergillus clavatus was used for amplifying the AclXyn5 gene for expression. The protein product of the AclXyn5 gene belongs to Glycosyl hydrolase family 11 based on the PFAM search (http://pfam.sanger.ac.uk/). At the N-terminus, AclXyn5 protein has an 18 amino acid signal peptide predicted by SignalP-NN (Emanuelsson et al., Nature Protocols, 2:953-971, 2007). This indicates that AclXyn5 is a secreted glycosyl hydrolase.

### Example 8

### Expression of AclXyn5protein (Xylanase B)

The AclXyn5 gene was amplified from genomic DNA of Aspergillus clavatus using the following primers: Primer 1(Not I) 5′-ccgcggccgcaccATGGTGTCGTTCAAGTATCTTTTCCT-3′ (SEQ ID NO: 27), and Primer 2 (Asc I) 5′-ccggcgcgcccttaTTAATAGACAGTAATGGAGGAGGAAC-3′ (SEQ ID NO: 28). After digestion with Not I and Asc I enzymes, the PCR product was cloned into pTrex3gM expression vector (described in US 2011/0136197 A1) digested with the same restriction enzymes, and the resulting plasmid was designated pZZH159. The map of plasmid pZZH159 is provided in FIG. 24. The sequence of the AclXyn5 gene was confirmed by DNA sequencing. The plasmid pZZH159 was transformed into a quad deleted Trichoderma reesei strain (described in WO 05/001036) using biolistic method (Te'o VS et al., J Microbiol Methods, 51:393-9, 2002).

Following sequence confirmation, protoplasts of a quad deleted T. reesei strain (described in WO 05/001036) were transformed with the expression plasmid pZZH159 using the PEG protoplast method (Penttila et al, Gene, 61:155-164, 1987). For protoplast preparation, spores were grown for about 10 hours at 24° C. in Trichoderma Minimal Medium MM (20 g/L glucose, 15 g/L KH₂PO₄, pH 4.5, 5 g/L (NH₄)2SO₄, 0.6 g/L MgSO₄×7H₂O, 0.6 g/L CaCl₂×2H₂O, 1 ml of 1000× T. reesei Trace elements solution (175 g/L Citric Acid anhydrous, 200 g/L FeSO₄×7H₂O, 16 g/L ZnSO₄×7H₂O, 3.2 g/L CuSO₄, 1.4 g/L MnSO₄×H2O, and 0.8 g/L Boric Acid). Germinating spores were harvested by centrifugation and treated with 30 mg/mL Vinoflow FCE (Novozymes, AG Switzerland) solution for from 7 hours to overnight at 30° C. at 100 rpm to lyse the fungal cell walls. Protoplasts were washed in 0.1 M Tris HCl buffer (pH 7) containing 0.6 M sorbitol and resuspended in 10 mM Tris HCl buffer (pH 7.5) containing 1.2 M sorbitol and 10 mM calcium chloride. For PEG transformation, approximately 1 μg of DNA and 1-5×10⁷ protoplasts in a total volume of 200 μl were treated with 2 ml of 25% PEG solution, diluted with 2 volumes of 1.2 M sorbitol/10 mM Tris, pH 7.5/10 mM CaCl₂ solution.

Transformants were selected on a medium containing acetamide as a sole source of nitrogen (acetamide 0.6 g/L; cesium chloride 1.68 g/L; glucose 20 g/L; potassium dihydrogen phosphate 15 g/L; magnesium sulfate heptahydrate 0.6 g/L; calcium chloride dihydrate 0.6 g/L; iron (II) sulfate 5 mg/L; zinc sulfate 1.4 mg/L; cobalt (II) chloride 1 mg/L; manganese (II) sulfate 1.6 mg/L; agar 20 g/L; pH 4.25). Transformed colonies (about 50-100) appeared in about 1 week. After growth on acetamide plates, the spores were collected and reselected on acetamide plates. After 5 days, the spores were collected using 10% glycerol, and 1×10⁸ spores were inoculated in a 250 ml shake flask with 30 ml Glucose/Sophorose defined medium for protein expression. Protein expression was confirmed by SDS-PAGE. The spore suspension was subsequently grown in a 7 L fermentor in a defined medium containing 60% glucose-sophorose feed. Glucose/Sophorose defined medium (per liter) consists of (NH₄)2SO₄ 5 g, PIPPS buffer 33 g, Casamino Acids 9 g, KH₂PO₄ 4.5 g, CaCl₂ (anhydrous) 1 g, MgSO₄.7H₂O 1 g, pH to 5.5 adjusted with 50% NaOH with Milli-Q H₂O to bring to 966.5 mL. After sterilization, the following were added: 26 mL 60% Glucose/Sophrose, and 400× T. reesei Trace Metals 2.5 mL.

AclXyn5 protein was purified from concentrated 7 L fermentor culture supernatant using two chromatography columns. Concentrated culture supernatant buffered in 20 mM sodium phosphate buffer pH 6.0 containing 1 M ammonium sulfate was loaded on a hydrophobic interaction chromatography column (Sepharose Butyl FF, XK 26/10). The protein was eluted from the column using a linear gradient of equilibration/wash buffer to 20 mM sodium phosphate buffer pH 6.0. The fraction containing the AclXyn5 protein was loaded onto a gel filtration column (HiLoad Superdex 75 μg 26/60), and the mobile phase used was 20 mM sodium phosphate, pH 7.0, containing 0.15 M NaCl. The purified protein was concentrated using a 3K Amicon Ultra-15 device and the concentrated protein fraction was used in further studies.

The nucleotide sequence of AclXyn5 gene from expression plasmid pZZH159 is set forth as SEQ ID NO:20. The signal sequence is shown in bold, and the predicted intron is shown in italics and lowercase.

The amino acid sequence of AclXyn5 protein expressed from plasmid pZZH159 is set forth as SEQ ID NO:17. The signal sequence predicted by SignalP-NN software is shown in italics. The amino acid sequence for the mature form of AclXyn5 protein as predicted by SignalP-NN software is set forth as SEQ ID NO:18. The amino acid sequence of a further processed mature form of the AclXyn5 protein is set forth as SEQ ID NO: 19 which could arise from posttranslational processing, e.g. Kexll N-terminal processing.

### Example 9

### Xylanase Activity of AclXyn5 (Xylanase B)

AclXyn5 belongs to the glycosyl hydrolase family 11 (based on the CAZy numbering system). The beta 1-4 xylanase activity of AclXyn5 was measured using 1% xylan from birch wood (Sigma 95588) or 1% arabinoxylan from wheat flour (Megazyme P-WAXYM) as substrates. The assay was performed in 50 mM sodium citrate pH 5.3, 0.005% Tween-80 buffer at 50° C. for 10 minutes.

The released reducing sugar was quantified by reaction with 3, 5-Dinitrosalicylic acid and measurement of absorbance at 540 nm. The enzyme activity is quantified relative to a xylose standard curve. In this assay, one xylanase unit (U) is defined as the amount of enzyme required to generate 1 micromole of xylose reducing sugar equivalents per minute under the conditions of the assay.

### Example 10

### Processes of Preparing Corn Based Foodstuff

Corn flour tortilla formulas and processing parameters used for this study, and which represent what is most commonly used in Mexico were as follows. Alkaline pH conditions are set out in Table 1 and acid pH conditions in Table 2.

TABLE 1

| | Amount (parts by weight |
|---|---|
| | based on 100 |
| Ingredients | parts corn flour weight) |
| Corn flour (from supplier GRUMA or | 100.00 |
| MINSA) | |
| Water (at 15° C.) | 114.00 |
| Calcium Hydroxide (solution at 5% w/v) | 16.00 |

TABLE 2

| | Amount (parts by |
|---|---|
| | weight based on 100 |
| Ingredients | parts corn flour weight) |
| Corn flour (from supplier GRUMA or | 100.00 |
| MlNSA) | |
| Water (at 15° C.) | 130.00 |
| Calcium propionate | 0.715 |
| Potassium sorbate | 0.500 |
| Monocalcium phosphate | 0.186 |
| Salt | 0.558 |
| Citric acid | 0.250 |

Processing conditions (the same processing conditions are used to obtain both acid and alkaline corn flour tortillas)

1) Scale and weigh all dry ingredients
2) In an N-150 Hobart mixer using a paddle, add liquid ingredients to the dry ingredients while mixing for one minute at 1^{st} speed.
3) Scrape bowl and paddle and continue mixing for additional 2 minutes on 2^{nd} speed.
4) Let the dough rest for 12 minutes at ambient temperature (approx 27° C.)
5) Process the corn dough through a tortilla machine “Superior Food Machinery Model CFO 440”
6) Raw corn tortilla weight 26 g, 14.5 cm in diameter; baking time 50 seconds at 246° C.

The finished corn tortillas were stacked in 10 tortillas per stack and packaged in a plastic bag. The tortilla packages were stored at ambient temperature (27° C.) during the shelf life period.

The xylanases were introduced into the corn flour together with other dry ingredients as a powder prepared as described in Example 11 below, at the following dosages:

A) Xylanase A1 at 0.08, 0.16 and 0.32 mg/kg corn flour
B) Xylanase B at 0.075, 0.15 and 0.30 mg/kg corn flour

As CMC (carboxymethylcellulose) is commonly used for corn flour tortilla production in Mexico, the above dosages of each of the enzymes were tested by themselves and in combination with GRINDSTED CMC MAS 550 (high viscosity carboxymethyl-cellulose commercially available from DuPont) at 0.50% by weight of the corn flour.

The performance of the two xylanases, was compared with that of GRINDSTED FSB 700 (a blend containing alpha amylase and hydrocolloid) and with that of POWERFlex 2205 (a blend containing alpha amylase and hydrocolloid)(commercially available from DuPont) which are DuPont commercial blends used for corn flour tortilla application, both of them were tested by themselves and in combination with GRINDSTED CMC MAS 550 at 0.50% by weight of corn flour.

### Example 11

### Preparation of the Xylanase Powders

Dry products are produced by spraying high-concentrated liquid ferment onto a carrier consisting of fine ground wheat. Ground wheat material has been heat treated with dry steam to reach a temperature of 103° C. for 30 seconds in order to enhance water absorption. 30-35% (w/w) liquid is applied with a peristaltic pump during mixing of the wheat carrier. After liquid application the material is spread onto trays and dried in circulating air for 10 hours at 40° C. The dry material is ground on a mill to produce a particle size of 200-1000 μm.

### Example 12

### Viscosity Evaluation of Corn Masa

To measure viscosity of the produced corn masa, a “slurry” was produced by mixing 150 grams of corn masa from both alkaline and acidic pH conditions in a high speed mixer with 150 grams of distilled water. The viscosity of the slurry was measured using a Brookfield LV Viscosimeter with a Spindle 4 at 30 rpm for 30 seconds.

The results are shown in Table 3 and FIG. 25 for alkaline pH and Table 4 and FIG. 26 for acidic pH. In each case, the amounts are expressed in % or ppm by weight of the corn flour. In all of these Examples, the term “ppm” is synonymous with “mg/kg flour”.

TABLE 3

| | Viscosity | Viscosity | Viscosity |
|---|---|---|---|
| | mPa · s at | mPa · s at | mPa · s at |
| Sample | 0 min | 15 min | 30 min |
| Control | 3140 | 3873 | 3868 |
| Control + 0.5% GRINDSTED | 2070 | 2452 | 2262 |
| CMC MAS 550 | | | |
| 0.32 Xylanase A1 | 4605 | 6038 | 6404 |
| 0.30 ppm Xylanase B | 4457 | 4760 | 6287 |
| 0.32 ppm Xylanase A1 + 0.5% | 3002 | 4525 | 5024 |
| GRINDSTED CMC MAS 550 | | | |
| 0.30 ppm Xylanase B + 0.5% | 3780 | 4391 | 5703 |
| GRINDSTED CMC MAS 550 | | | |

TABLE 4

| | Viscosity | Viscosity | Viscosity |
|---|---|---|---|
| | mPa · s at | mPa · s at | mPa · s at |
| Sample | 0 min | 15 min | 30 min |
| Control | 1200 | 1500 | 1600 |
| CMC (0.5%) | 2800 | 4000 | 5000 |
| Xylanase B (0.3 ppm) + CMC (0.5%) | 3800 | 5100 | 6000 |

The results presented in Tables 3 and 4, in which viscosity was measured to a “slurry” produced by mixing 150 grams of corn masa (both with alkaline or acidic pH conditions) with 150 grams of distilled water, reveal that a combination of Xylanase B at 0.3 ppm+GRINDSTED CMC MAS 550 at 0.5% and a combination of Xylanase A1 at 0.32 ppm+GRINDSTED CMC MAS 550 at 0.5% in the corn masa significantly increases the viscosity of the slurry compared to control and with GRINDSTED CMC MAS 550 alone. Also the addition of Xylanase A1 at 0.32 ppm and Xylanase B at 0.3 ppm increases viscosity over control under alkaline conditions. a combination of Xylanase B at 0.3 ppm+GRINDSTED CMC MAS 550 at 0.50% in the corn masa, significantly increases the viscosity of the slurry compared to the viscosity produced by adding Xylanase A1 at 0.32 ppm by itself or in combination with GRINDSTED CMC at 0.50% or by adding GRINDSTED CMC MAS 550 at 0.50% by itself to the corn masa.

### Example 13

### Flexibility Test

Tortillas were produced as described above. Tortillas produced under alkaline pH and acidic pH conditions were produced with the mixtures as indicated in Tables 5 (alkaline pH) and 6 (acidic pH). The amounts are expressed in % by weight, relative to 100% weight of the corn flour, or ppm (=mg/kg corn flour).

TABLE 5

| | Sample No | | | | |
|---|---|---|---|---|---|
| | 1 | 2 | 3 | 4 | 5 |
| Xylanase B (ppm) | 0.3 | 0 | 0 | 0 | 0 |
| GRINDSTED CMC MAS 550 (%) | 0.50 | 0.50 | 0.50 | 0.50 | 0 |
| GRINDSTED FSB 700 (%) | 0 | 0 | 0.375 | 0 | 0 |
| POWERFlex 2205 (%) | 0 | 0 | 0 | 0.13 | 0 |

TABLE 6

| | Sample No | | | | |
|---|---|---|---|---|---|
| | 1 | 2 | 3 | 4 | 5 |
| Xylanase A1 (ppm) | 0.16 | 0.32 | 0 | 0 | 0 |
| GRINDSTED CMC MAS 550 (%) | 0.50 | 0.50 | 0.50 | 0.50 | 0.50 |
| GRINDSTED FSB 700 (%) | 0 | 0 | 0 | 0.375 | 0 |
| POWERFlex 2205 (%) | 0 | 0 | 0 | 0 | 0.13 |

Pictures showing the foldability of the corn tortillas with each of the variables tested were taken at day 10 after production according to the following procedure:

The tortillas were folded in half when cold and a weight of 100 g was placed on top of the tortilla for 30 seconds, then the picture was taken.

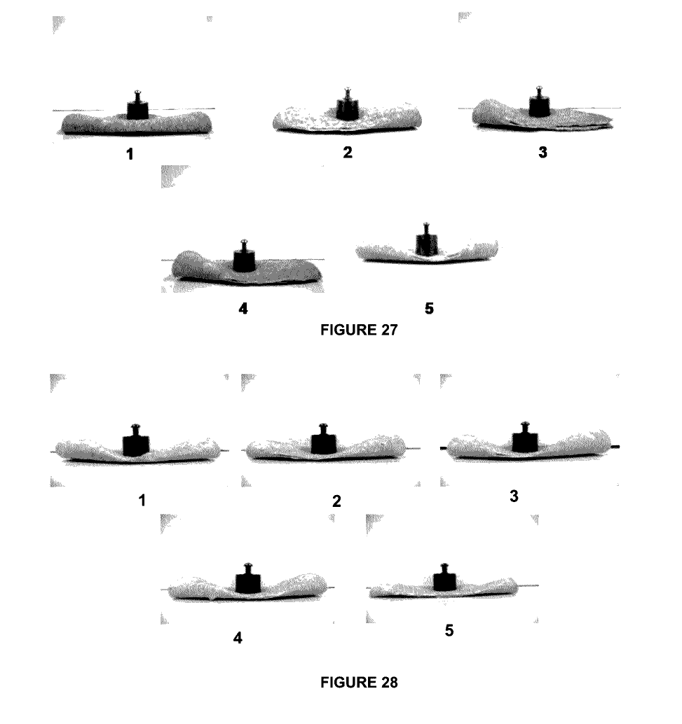

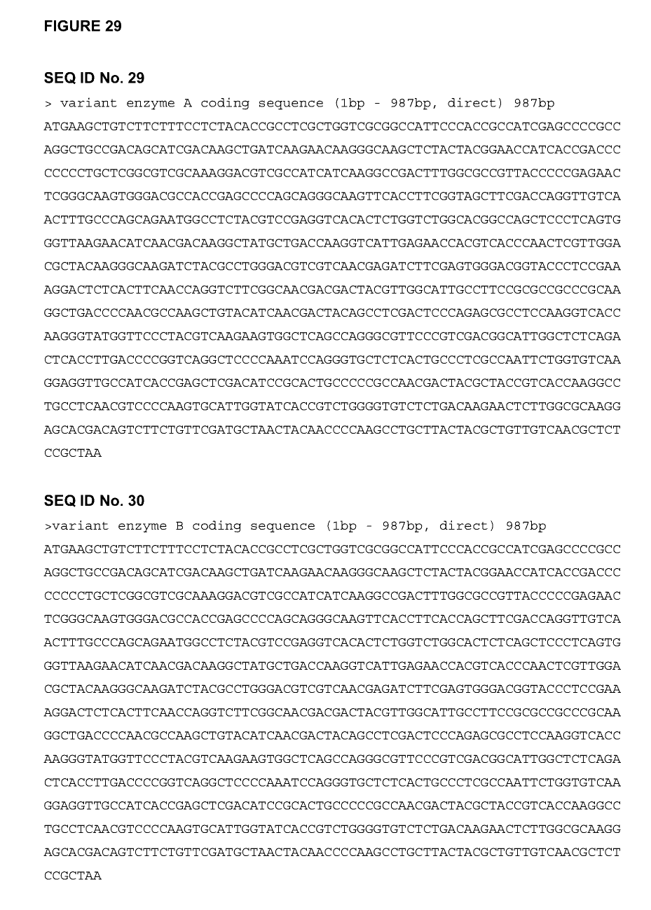

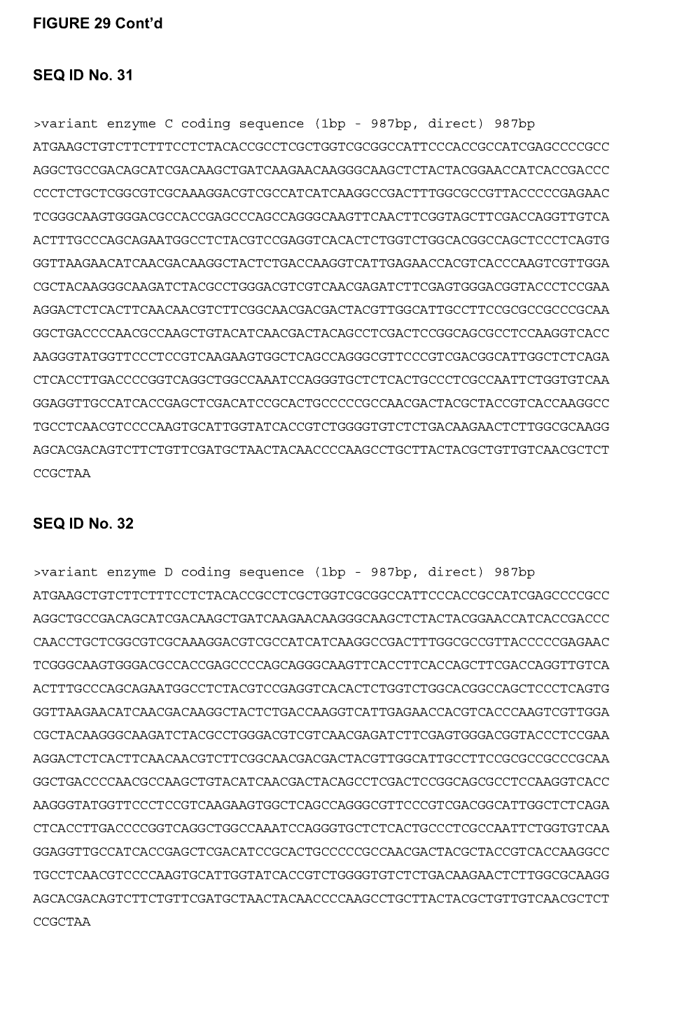

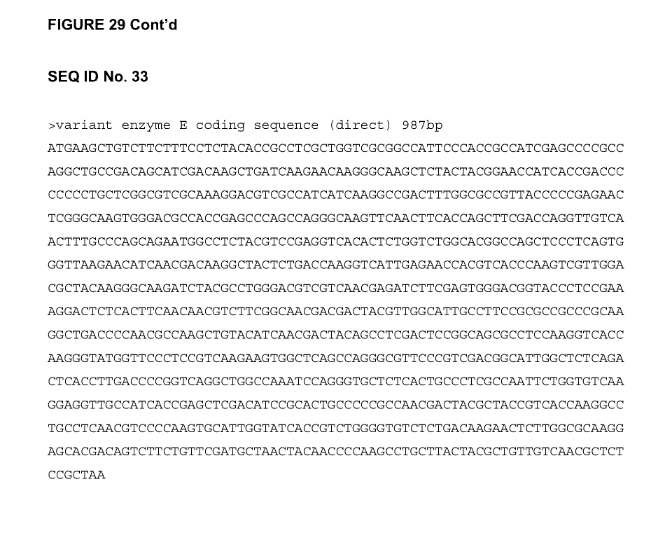

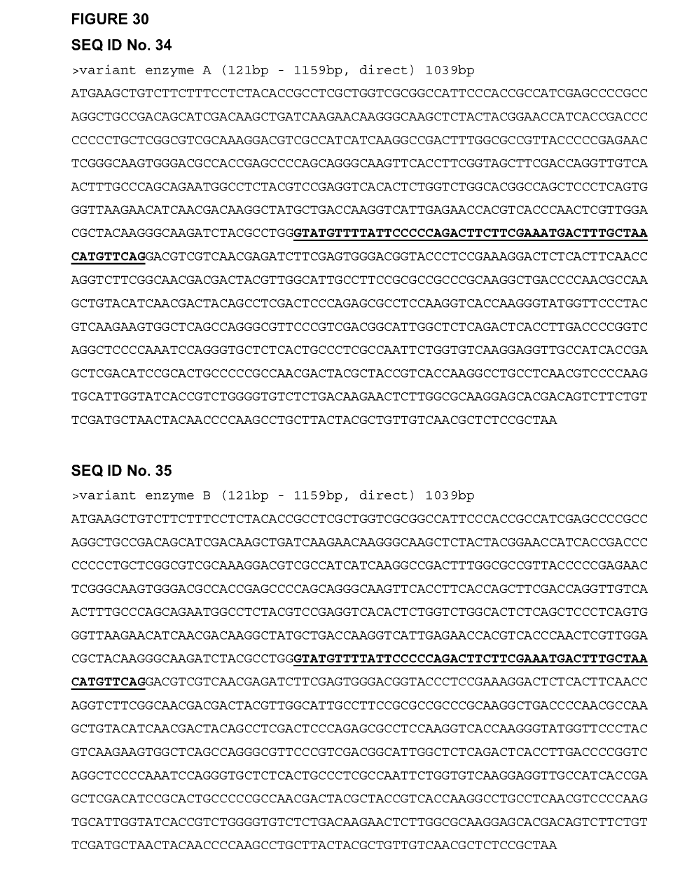

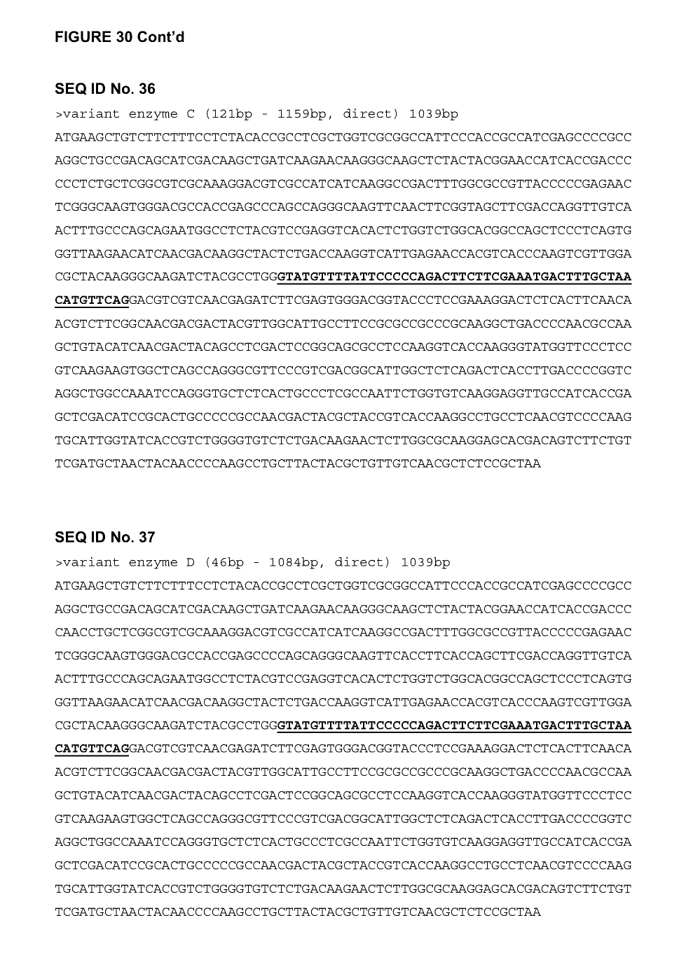

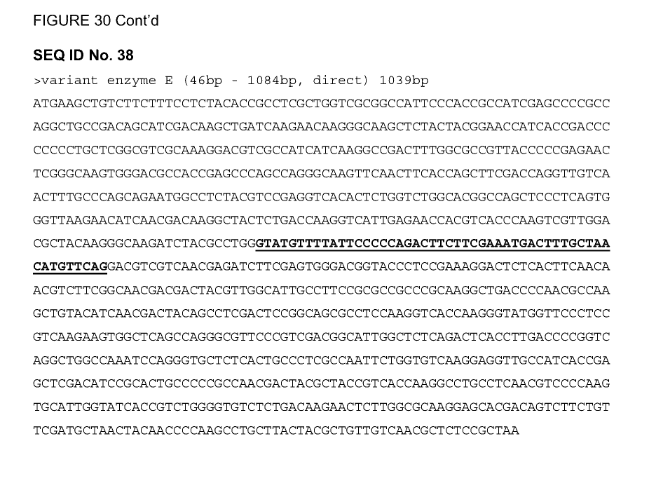

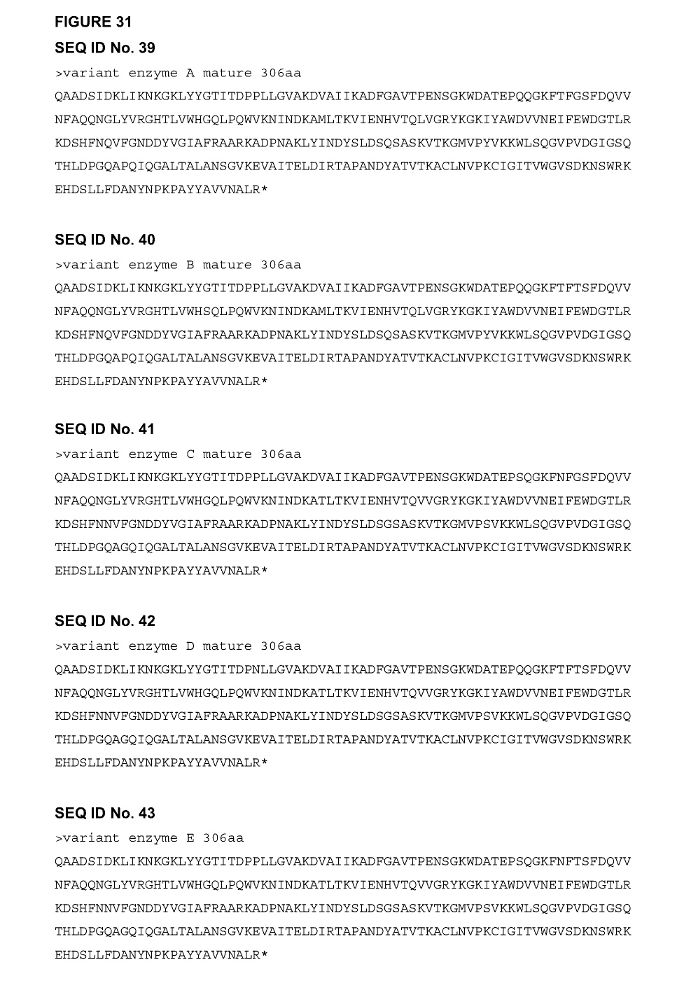

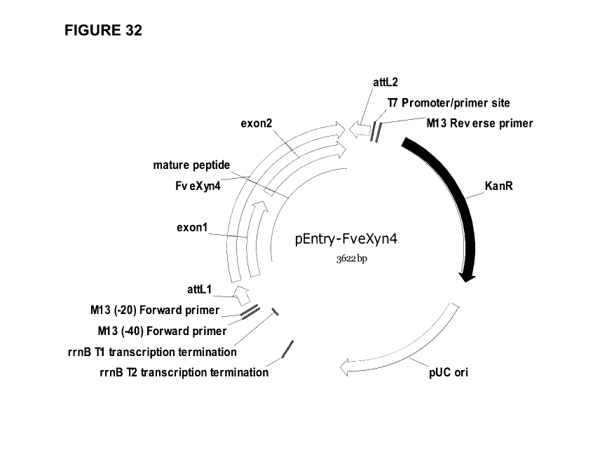

The results are shown in FIG. 27 (alkaline pH) in which the mixtures are as indicated in Table 5, and FIG. 28 (acidic pH) in which the mixtures are as indicated in Table 6:

The results presented in FIGS. 27 and 28, show that after carrying out the procedure above, indicate that after 10 days of their shelf life the combination of Xylanase B at 0.3 ppm+GRINDSTED CMC MASS 550 at 0.50% produces the more flexible and resistant tortilla in comparison to any of the other variables tested in alkaline conditions (FIG. 27) and that the combination of Xylanase A1 at 0.16 ppm+GRINDSTED CMC MAS 550 at 0.50% produces the more flexible and resistant tortilla in comparison to any of the other variables tested in acidic conditions (FIG. 28).

In addition to the above, the surface texture of the tortillas, with both of the Xylanases A1 or B, feels to the touch significantly softer in comparison to any of the other variables tested including GRINDSTED FSB 700 and POWERFlex 2205.

### Example 14

### Sensory Evaluation Data

Corn flour tortillas were prepared as set out in Table 7 below according to the following procedure:

1) Scale all dried ingredients
2) In an N-50 Hobart mixer using a paddle, add liquid ingredients to the dried ingredients while mixing for one minute at 1^{st} speed.
3) Continue mixing for additional 2 minutes on 2^{nd} speed.
4) Let the dough rest for 12 minutes at ambient temperature (approx 27° C.)
5) Process the corn dough through a tortilla machine “Superior Food Machinery Model CFO 440”
6) Raw corn tortilla weight 26 g, 14.5 cm in diameter; baking time 50 seconds at 246° C.

In Table 7, the amounts are expressed in % or ppm by weight relative to 100 weight of the corn.

TABLE 7

| Samples | 1 | 2 | 3 | 4 | 5 | 6 |
|---|---|---|---|---|---|---|
| Corn flour MASECA | 100.00 | 100.00 | 100.00 | 0 | 0 | 0 |
| (GRUMA) (%) | | | | | | |
| Corn flour MINSA | 0 | 0 | 0 | 100.00 | 100.00 | 100.00 |
| (%) | | | | | | |
| Water (at 15° C.) (%) | 105.22 | 105.22 | 105.22 | 105.22 | 105.22 | 105.22 |
| Calcium Hydroxide | 14.78 | 14.78 | 14.78 | 14.78 | 14.78 | 14.78 |
| (solution at 5% w/v) | | | | | | |
| (%) | | | | | | |
| Salt (%) | 0.5 | 0.5 | 0.5 | 0.5 | 0.5 | 0.5 |
| GRINDSTED CMC | 0.5 | 0.5 | 0.5 | 0.5 | 0.5 | 0.5 |
| MAS 550 (%) | | | | | | |
| Xylanase B (ppm) | 0 | 0.3 | 0 | 0 | 0.3 | 0 |
| Xylanase A1(ppm) | 0 | 0 | 0.32 | 0 | 0 | 0.32 |

After 1 day shelf life the Samples 1, 2 and 3 were tested for resistance and samples 4, 5 and 6 for texture according to the procedure below:

Resistance (how much the tortilla resists a tough handling)—a tortilla was taken out of the package; then placed on top of the hand and squeezed by closing the hand into a fist, after this the hand opened and observe how much the tortilla cracked. The resistance was assessed on a preference scale of 1 (Not resistant at all—the tortilla is fully cracked) to 9 (Very resistant—the tortilla remains in shape).
Texture (how soft the tortilla felt to the touch)—:a tortilla was taken out of the package; then the surface of the tortilla felt by touching it with the fingers, the texture recorded on a preference scale ranging from 1 (Very rough and stiff) to 9 (very soft and velvety).

The results are shown in Tables 8 and 9 below.

TABLE 8

| | Sample | | |
|---|---|---|---|
| | 1 | 2 | 3 |
| Xylanase | CoZntrol | Xylanase B | Xylanase A1 |
| Average | 2.3 | 4.4 | 3.9 |
| resistance score | | | |

The results in Table 8 show that both Sample 2, containing Xylanase B and Sample 3, containing Xylanase A1, are significantly more resistant than Sample 1 (control).

TABLE 9

| | Sample | | |
|---|---|---|---|
| | 4 | 5 | 6 |
| Xylanase | Control | Xylanase B | Xylanase A1 |
| Average | 4.1 | 5.5 | 6.5 |
| texture score | | | |

The results in Table 9 show that both Sample 5, containing Xylanase B, and Sample 6, containing Xylanase A1, are significantly softer than Sample 4 (control).

### Example 15

### Texture and Flexibility Test

Corn flour tortillas were prepared as follows:

The formula and processing conditions for the corn flour tortilla were as follows:

Ingredients % (based on 100% corn flour)
Commercial GRUMA corn flour with 0.50% of Cellulose Gum 100.00% Water (at 15° C.) 114.00%
Calcium Hydroxide (solution at 10% w/v, ie 10 g of calcium hydroxide in 90 ml of water) 16.00%

### Salt 0.55%

Xylanase A1 and Xylanase B were added at the below doses:

1) Xylanase A1 at 0.16 mg/kg of corn flour
2) Xylanase B at 0.15 mg/kg of corn flour

Process:

1) Scale and weigh all dried ingredients
2) In an N-150 Hobart mixer using a paddle, add liquid ingredients to the dried ingredients while mixing for one minute at 1st speed.
3) Scrape bowl and paddle and continue mixing for additional 2 minutes on 2nd speed.
4) Let the dough rest for 12 minutes at ambient temperature (approx 27° C.)
5) Process the corn dough through a tortilla machine “Superior Food Machinery Model CFO 440”
6) Raw corn tortilla weight 26 g, 5.8 inches in diameter; baking time 50 seconds at 246° C.

The performance of the xylanases A1 and B was compared with that of GRINDSTED FSB 700 dosed at 0.375% which is the actual blend of ingredients used for corn flour tortilla application; both of the xylanases A1 and B provided an improved flexibility and an improved resistance to the corn tortilla than GRINDSTED FSB 700 did over a shelf life of 20 days stored at ambient temperature; the surface texture of the tortillas with both xylanases A1 and B was softer, with a more “velvety texture” than the tortillas with GRINDSTED FSB 700, those are positive attributes in a corn flour tortilla.

A very positive attribute, as seen in the results, is that both Xylanase A1 and Xylanase B, but especially Xylanase B, demonstrate a good performance at pH in between 10 to 11, which is the pH of the tortillas produced with “nixtamalized” corn, (treatment with calcium hydroxide); the majority of the corn tortillas produce in Mexico are from nixtamalized corn.

### Example 16

### Preparation of a Thermostable Xylanase (Xylanase C)

### Materials and Methods

### Plasmid and Library Construction

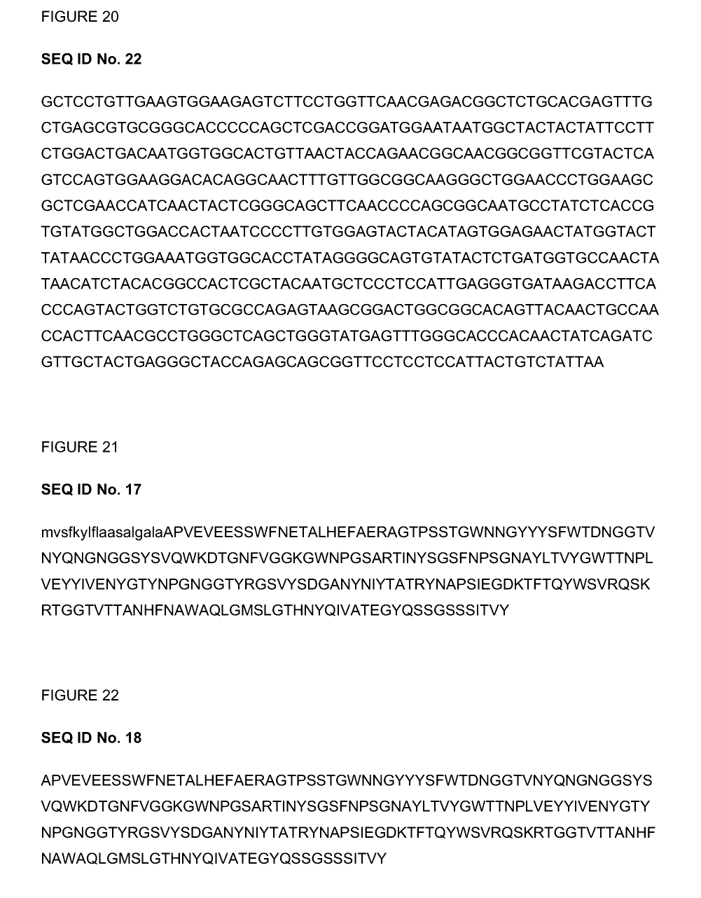

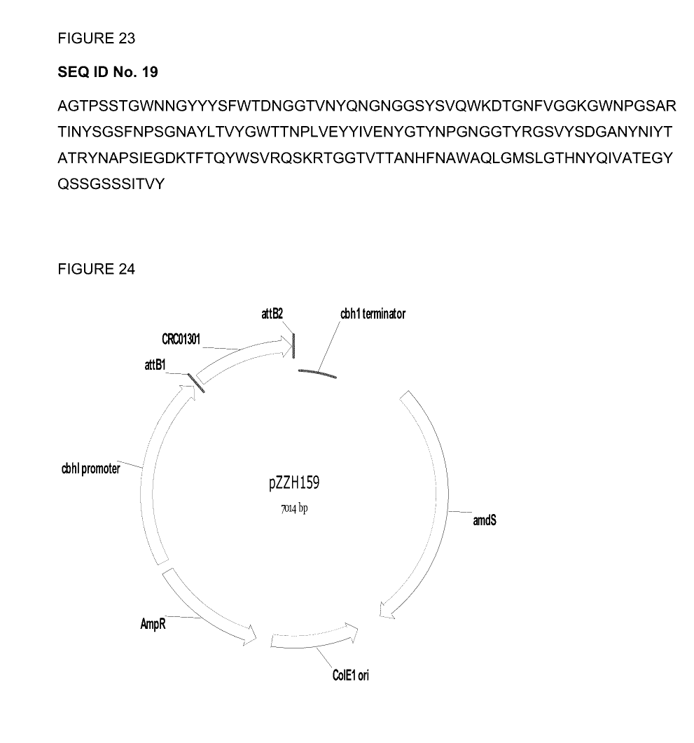

A DNA sequence containing the coding region for xylanase 4 (the family GH10) from the filamentous fungus Fusarium verticilloides, FveXyn4, was amplified from the genomic DNA with the gene specific primers extended with the attB1 and attB2 sites to allow for the Gateway® BP recombination cloning into the pDonor221 vector (Invitrogen, USA). The pEntry-FveXyn4 plasmid, as shown in FIG. 20 was used by the vendors BaseClear (Netherlands) and Geneart GmH (Germany) as template for construction of combinatorial libraries.

Variants of FveXyn4 was generated either as combinatorial libraries or by introduction of specific mutations and were designed to included different numbers and combinations of the mutations presented in Table 1. Variant A, B, C, D, and E were included in these variants.

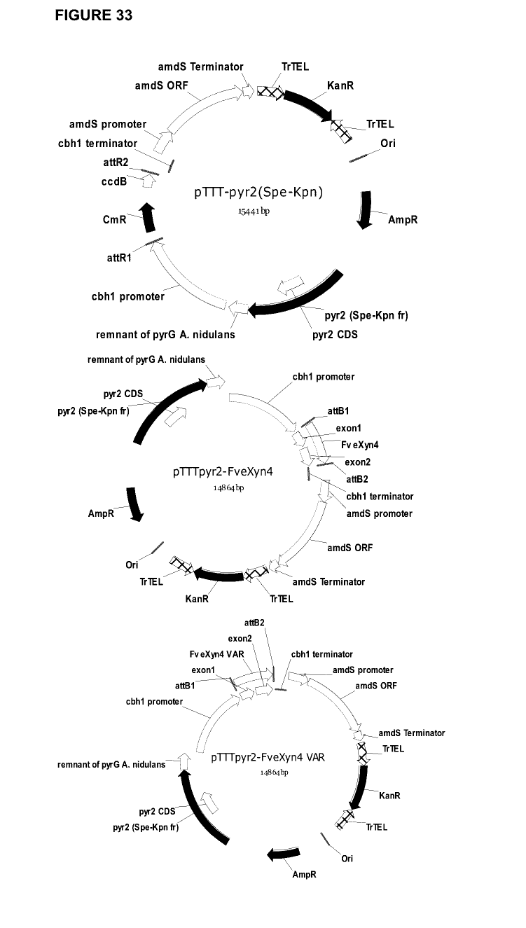

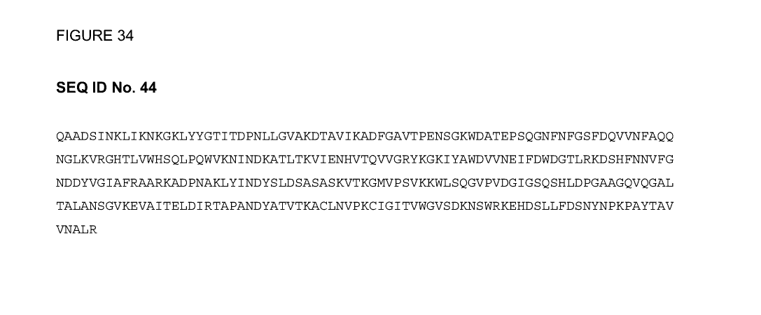

Combinatorial variants were generated via the Gateway® recombination technique (Invitrogen, USA) with the destination vector pTTTpyr2 (FIG. 33). The resulting expression plasmids pTTTpyr2-FveXyn4_VAR expressing Xyn4 with different mutations were amplified in the Escherichia coli DH5a strain, purified, sequenced, arrayed individually in 96 MTPs and used for fungal transformation as described further. The expression vector contains the T. reesei cbhl promoter and terminator regions allowing for a strong inducible expression of a gene of interest, the Aspergillus nidulans amdS and T. reesei pyr2 selective markers conferring growth of transformants on minimal medium with acetamide in the absence of uridine. The plasmids are maintained autonomously in the fungal cell due to T. reesei derived telomere regions. Usage of replicative plasmids results in increased frequencies of transformation and circumvents problems of locus-dependent expression observed with integrative fungal transformation.

Specific mutations were introduced into the genomic sequence of the Fusarium verticilloides xylanase Xyn4 via a de novo gene synthesis (GeneArt GmbH, Germany). Synthetic variants were then cloned by the vendor into the destination vector pTTT-pyr2 via a Gateway recombination technique (Invitrogen, Carlsbad, Calif., USA).

Fungal Strains, Growth Media and Transformation Expression plasmids (5-10 μl) were transformed using a PEG-protoplast method into a T. reesei strain deleted for major cellulases and xylanase 2 (Δcbh1 Δcbh2 Δegl1 Δegl2 Δegl3 Δegl4 Δegl5 Δegl6 Δbgl1 Δman1 Δxyn2 Prdiv: iRNAxyn1 xyn3: amdS pyr2-). Additional downregulation of the endogenous xylanase 1 and 3 background was further achieved via introducing into the host strain genome an iRNA interference cassette targeting to shut down the xyn1 and xyn3 expression simultaneously. All high throughput transformations were performed robotically in a 24 well MTP format using Biomek robots (Beckman Coulter, USA). Plasmids with variants were received from the vendors in 96 well MTPs arrayed according to a predetermined layout. Transformation mixtures containing approximately 1 μg of DNA and 5×10⁶ protoplasts in a total volume of 50 μl were treated with 200 μl of 25% PEG solution, diluted with 1 volumes of 1.2M sorbitol/10 mM Tris, pH7.5/10 mM CaCl₂ solution, rearranged robotically into 24 well MTPs and mixed with 1 ml of 3% agarose Minimal Medium containing 1M sorbitol and 10 mM NH4Cl. After growth of transformants, spores from each well were pooled and repatched on fresh 24 well MTPs with MM containing acetamide for additional selective pressure. Once sporulated, spores were harvested and used for inoculation of liquid cultures either in a 24-well MTP format or shake flasks in the following production medium: 37 g/L glucose, 1 g/L sophorose, 9 g/L casmino acids, 10 g/L (NH₄)₂SO₄, 5 g/L KH₂PO₄, 1 g/L CaCl₂×2H₂O, 1 g/L MgSO₄×7H₂O, 33 g/L 1,4-Piperazinebis(propanesulfonic acid), pH 5.5, 2.5 ml/L of 400× T. reesei trace elements (175 g/L citric acid, 200 g/L FeSO4×7H₂O, 16 g/L ZnSO4×7H₂O, 3.2 g/L CuSO4×5H₂O, 1.4 g/L MnSO4×H₂O, 0.8 g/L boric acid). 1 ml of production medium was added to produce variants in 24 well MTPs. For shake flasks, volumes were scaled up.

Plates were grown for 6 days at 28° C. and 80% humidity with shaking at 200 rpm. Culture supernatants were harvested by vacuum filtration and used to assay their performance as well as expression level.

For larger scale production fermentation in a 6 Liter autoclaveable Continuers Stirred Reactor was conducted. Shake flasks were inoculated with spores and incubated with shaking for 3 days at 28° C. in the following shake flask medium: 5 g/L (NH₄)₂SO₄, 4.5 g/L KH₂PO₄, 1 g/L MgSO₄×7H₂O, 14.4 g/L citric acid ×1H₂O, 1 g/L CaCl₂×2H₂O, 27.5 g/L glucose, 1 drop antifoam agent (EROL DF 6000K). The pH was adjusted with NaOH (2M) to 5.5 and media was autoclaved 20 minutes at 122° C. After cooling 2.5 ml/L of 400× T. reesei trace elements (175 g/L citric acid, 200 g/L FeSO4×7H₂O, 16 g/L ZnSO4×7H₂O, 3.2 g/L CuSO4×5H₂O, 1.4 g/L MnSO4×H₂O, 0.8 g/L boric acid) was added. Cells from the shake flask was used to inoculate the bioreactor containing the following Bioreactor medium: 4.7 g/L KH₂PO₄, 1 g/L MgSO₄×7H₂O, 4.3 g/L (NH₄)₂SO₄, 45 g/L glucose, 0.7 g/L CaCl₂×2H₂O, 0.3 g/L antifoam agent (EROL DF 6000K), 2.5 ml/L of 400× T. reesei trace elements (175 g/L citric acid, 200 g/L FeSO4×7H₂O, 16 g/L ZnSO4×7H₂O, 3.2 g/L CuSO4×5H₂O, 1.4 g/L MnSO4×H₂O, 0.8 g/L boric acid). Temperature was controlled at 34° C.; pH was continuously controlled by adding 20% ammoniumhydroxide. Dissolved oxygen was controlled to minimum 40% saturation by varying the stirring rate. Off gas carbon dioxide and oxygen content were measured. When the initial glucose was depleted a constant feeding of a glucose/sophorose was started. At the same time temperature was reduced to and controlled at 28° C., pH was increased to and controlled at 4.5. The fermentation was terminated after 140 hours. Broth was removed from the tank, and cells were removed by filtration. After cell separation the filtrate was concentrated by ultrafiltration. Finally, the concentrate was sterile filtered and used for pelleting stability studies.

### Enzyme Samples

Xylanase activity of culture supernatants from MTP were measured using the method for measurement of xylanase activity as described below. Culture supernatants were diluted 20 and 130 times in 25 mM sodium acetate, 250 mM NaCl, pH 4.0. 25 μL diluted enzyme sample was mixed with 150 μL 0.5% WE-AX substrate, pH 5.0 and incubated at 30° C. for 15 min with shaking. After incubation, 45.4 μL reaction sample was mixed with 135 μL PAHBAH working solution and incubated at 95° C. for 5 min before cooled to 20° C. for 10 sec. 100 μL sample was transferred to a microtiter plate well and the plate was read at 410 nm.

The activity was calculated as the mean of three replicates subtracted a blank including 25 mM sodium acetate, 250 mM NaCl, pH 4.0 instead of enzyme. Protein concentration of the samples were calculated based on a standard curve of purified FveXyn4. All samples were diluted to 50 ppm in 25 mM sodium acetate, 250 mM NaCl, pH 4.0. These normalised samples were used as enzyme stock solution in assays described below.

Protein concentration in the enzyme stock solution was measured by HPLC as described below.

Xylanase activity of sterile filtered concentrates from large scale production was measured by the following activity assay. 0.5 g of each concentrate was weighed in 100 ml volumetric flasks followed by filling to volume with McIlvaine buffer, pH 5.0. Samples were diluted to app. 6 XU/ml using McIlvaine buffer, pH 5.0. 100 μl of diluted sample was added to 1 ml of McIlvaine buffer, pH 5.0 in test tubes and equilibrated at 50° C. for 2 min. A Xylazyme tablet (100 mg) was added to initiate the reaction and samples were incubated at 40° C. for 10 min before the reaction was stopped by adding 10 ml of 2% Tris, pH 12.0. The solution was mixed using vortex, left to stand for 5 min and mixed again before centrifuged at 3500 rpm for 10 min. Absorbance of the supernatant was measured at 590 nm. Each sample was measured in duplicate. Xylanase activity was quantified relatively to an enzyme standard (Danisco Xylanase, available from Danisco Animal Nutrition).

The benchmark enzyme Econase® XT is a commercially available and was extracted from commercial dry formulated samples. The xylanase component from Econase® XT commercial dry formulated samples was extracted in a 33% (w/w) slurry using McIlvain buffer, pH 5.0. The extract was cleared using centrifugation (3000 RCF for 10 min) and filtered using a PALL Acrodisc PF syringe filter (0.8/0.2 μm Supor membrane) and subsequently heated 20 min at 70° C. After removable of precipitation by centrifugation (38 724 RCF for 15 min) the buffer was replaced by passage through a Sephadex G25 column (PD10 from Pharmacia) equilibrated with 20 mM Na Citrate, 20 mM NaCl, pH 3.4. Purification of the xylanase component was performed using Source 15S resin, followed by elution with a linear increasing salt gradient (NaCl in 20 mM Na Citrate buffer pH 3.4).

Econase XT® is an endo-1,4-β-xylanase (EC 3.2.1.8) produced by the strain Trichoderma reesei RF5427 (CBS 114044), available from ABVista.

Protein concentration was determined by measuring absorption at 280 nm. The extinction coefficients were estimates from the amino acid sequences. For Econase XT the absorption at 280 nm of 1 mg/ml was calculated to be 2.84 AU.

TABLE 10
Overview of mutations in the five variants of FveXyn4

| Variants | Mutations |
|---|---|
| A | N7D_N25P_T33V_S57Q_N62T_K79Y_S89G_T103M_V115L_N147Q_G181Q_S193Y_A217Q_G219P_T298Y |
| B | N7D_N25P_T33V_S57Q_N62T_G64T_K79Y_T103M_V115L_N147Q_G181Q_S193Y_A217Q_G219P_T298Y |
| C | N7D_N25P_T33V_K79Y_S89G_A217Q_T298Y |
| D | N7D_T33V_S57Q_N62T_G64T_K79Y_S89G_A217Q_T298Y |
| E | N7D_N25P_T33V_G64T_K79Y_S89G_A217Q_T298Y |

Numbering is based on the mature sequence of FveXyn4. SEQ ID No. 3.

### Example 18

### Evaluation of Dough with Xylanase

A buffered dough was prepared using the ingredients listed in Table 11. Nixtamalized corn flour of the brand name MINSA was purchased from Mexican Supermarket. CMC MAS 550 was obtained from DuPont.

TABLE 11

| Ingredient | g per 100 g flour* | grams |
|---|---|---|
| Nixtamalized corn flour | 100 | 75 |
| 50 mM Phosphate buffer pH 6.0 | 130 | 97.5 |
| CMC MAS 550 | 0.5 | 0.375 |
| Xylanase A1 | 0.04 to 0.64 mg per | |
| | 1000 g flour | |

*all ingredient concentrations except Xylanase A1 are listed as g per 100 g flour

All Ingredients were equilibrated at 40° C. prior to use. Mixing was performed using a Kenwood kitchen Chef mixer fitted with a K beater. Dry ingredients were added to the mixing bowl and mixed briefly in speed 1. Liquid ingredients were added and mixing was done for 30 seconds in speed 1 and 30 seconds in speed 3. After end mix, the dough was moulded by hand for 10 seconds to form a round dough piece. Dough was rested for 30 minutes at 40° C. After 30 minutes of resting, a 5 g piece of dough was taken and frozen in liquid N₂. After storage (optional, at −18° C.), dough sample was added 10 g of ice cold deionised water and the mixture was homogenised using Ultra Turrax at 5-10000 rpm for 20 seconds followed by at 25000 rpm for 40 seconds. The weight of the tube (tarred) plus slurry was recorded to estimate the actual amount of homogeneous slurry used for centrifugation. The slurry was centrifuged at 4700 rpm at 10° C. for 15 minutes. The supernatant was decanted away from pellet and filtered through a glass filter. Soluble pentose content was determined as described in Example 19 below. Total weight of pellet and tube (tarred) was recorded.

Water holding capacity (WHC) was calculated as amount of pellet based on dough weight, and was calculated according to WAI according to the formula below (L.C. Platt-Lucero et al.: Journal of Food Process Engineering 2013, 36, 179-186), with the modification of using dough instead of flour dry matter as base.

$$\frac{Weight of pellet}{weight of dough} = \frac{g pellet \times ( g dough taken for slurry + g water for slurry )}{g slurry used for centrifugation \times g dough taken for slurry}$$

A WHC value of 1 would imply no extra absorption of water. Numbers below 1 would imply that some of the water added during dough preparation is eliminated from the dough during homogenization and centrifugation. Values above 1 imply that dough absorbs extra water compared to the recipe when homogenized in excess water.

The results are shown in Table 12, in which “ppm” means mg of the xylanase per kg of flour. As can be seen, the results in Table 12 show a 6% increase in water holding capacity over no enzyme added control.

TABLE 12

| | Water holding capacity |
|---|---|
| | (g pellet/g dough) |
| Dough formulation | T = 30 min |
| Control, with CMC | 1.25 |
| CMC + Xylanase A1 at 0.04 ppm | 1.28 |
| CMC + Xylanase A1 at 0.08 ppm | 1.28 |
| CMC + Xylanase A1 at 0.16 ppm | 1.33 |
| CMC + Xylanase A1 at 0.32 ppm | 1.33 |
| CMC + Xylanase A1 at 0.64 ppm | 1.33 |

### Example 19

### Quantification of C5 Sugars (Pentosans)

The total amount of pentoses brought into solution in dough liquor was measured using the method of Rouau and Surget, Carbohydrate Polymers, 1994, 24, 123-32 with a continuous flow injection apparatus. The supernatants were treated with acid to hydrolyse polysaccharides to monosugars. Phloroglucinol (1, 3, 5-trihydroxybenzene) was added for reaction with monopentoses and monohexoses, which forms a coloured complex.

By measuring the difference in absorbance at 550 nm compared to 510 nm, the amount of pentoses in the solution was calculated using a standard curve. Unlike the pentose-phloroglucinol complex, the absorbance of the hexose-phloroglucinol complex is constant at these wavelengths. Glucose was added to the phloroglucinol solution to create a constant glucose signal and further ensure no interference from hexose sugars. Amounts were expressed in ppm pentose. The results are shown in Table 13.

TABLE 13

| | Soluble pentose |
|---|---|
| Dough formulation | (ppm) T = 30 min |
| Control, with CMC | 528 |
| CMC + Xylanase A1 at 0.04 ppm | 555 |
| CMC + Xylanase A1 at 0.08 ppm | 615 |
| CMC + Xylanase A1 at 0.16 ppm | 647 |
| CMC + Xylanase A1 at 0.32 ppm | 1100 |
| CMC + Xylanase A1 at 0.64 ppm | 1253 |

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A process for the preparation of a corn-based foodstuff, the process comprising the step of contacting a corn-based flour with a xylanase enzyme, such that a xylan-containing material native to the corn is degraded;
wherein the xylanase enzyme is selected from the group consisting of:
(B) a polypeptide as set forth in SEQ ID No. 17 or SEQ ID No. 18 or SEQ ID No. 19; or a variant, fragment, homologue or derivative thereof having at 90% identity with SEQ ID No. 17 or SEQ ID No. 18 or SEQ ID No. 19; or encoded by a nucleotide sequence set forth as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 90% identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence set forth as SEQ ID No. 1, SEQ ID No. 2 or SEQ ID No. 3, or a variant, homologue, fragment or derivative thereof having at least 90% identity with SEQ ID No. 1, SEQ ID No. 2 or SEQ ID No. 3 or a polypeptide sequence which comprises SEQ ID No. 1, SEQ ID No. 2, or SEQ ID No. 3, with a conservative substitution of at least one of the amino acids, or is encoded by a nucleotide sequence set forth as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 90% identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of the following positions 7, 33, 79, 217 and 298, wherein the numbering is based on the amino acid numbering of FveXyn4 (SEQ ID No. 3) or
(A2) a polypeptide sequence set forth as SEQ ID No. 7, SEQ ID No. 8 or SEQ ID No. 9, or a variant, homologue, fragment or derivative thereof having at least 90% identity with SEQ ID No. 7, SEQ ID No. 8 or SEQ ID No. 9, or a polypeptide sequence which comprises SEQ ID No. 7, SEQ ID No. 8 or SEQ ID No. 9 with a conservative substitution of at least one of the amino acids; or is encoded by a nucleotide sequence set forth as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 90% identity with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

2. The process according to claim 1, wherein the corn-based flour at least partially comprises a nixtamalised corn flour.

3-6. (canceled)

7. The process according to claim 1 or claim 2 wherein a further enzyme is present in addition to the mixture of corn-based flour and xylanase enzyme.

8. The process according to claim 1 or claim 2 wherein a hydrocolloid is present in addition to the mixture of corn-based flour and xylanase enzyme.

9-11. (canceled)

12. The process according to claim 8 in which the hydrocolloid is carboxymethylcellulose.

13. The process according to claim 1 or claim 2, further comprising adding water to the mixture of corn-based flour and xylanase enzyme to form a masa.

14. The process according to claim 13, further comprising processing the masa into a masa foodstuff selected from corn tortilla, soft tortilla, corn chips, tortilla chips, taco shells, tamales, derivatives and mixtures thereof.

15-22. (canceled)

23. A corn-based flour comprising a xylanase enzyme, wherein the xylanase enzyme is selected from the group consisting of
(B) a polypeptide as set forth in SEQ ID No. 17 or SEQ ID No. 18 or SEQ ID No. 19; or a variant, fragment, homologue or derivative thereof having at least 90% identity with SEQ ID No. 17 or SEQ ID No. 18 or SEQ ID No. 19; or encoded by a nucleotide sequence set forth as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 90% identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence set forth as SEQ ID No. 1, SEQ ID No. 2 or SEQ ID No. 3, or a variant, homologue, fragment or derivative thereof having at least 90% identity with SEQ ID No. 1, SEQ ID No. 2 or SEQ ID No. 3, or a polypeptide sequence which comprises SEQ ID No. 1, SEQ ID No. 2, or SEQ ID No. 3, with a conservative substitution of at least one of the amino acids, or is encoded by a nucleotide sequence shown herein as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 90% identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of the following positions 7, 33, 79, 217 and 298, wherein the numbering is based on the amino acid numbering of FveXyn4 (SEQ ID No. 3) or
(A2) a polypeptide sequence set forth as SEQ ID No. 7, SEQ ID No. 8, or SEQ ID No. 9, or a variant, homologue, fragment or derivative thereof having at least 90% identity with SEQ ID No. 7, SEQ ID No. 8 or SEQ ID No. 9, or a polypeptide sequence which comprises SEQ ID No. 7, SEQ ID No. 8 or SEQ ID No. 9 with a conservative substitution of at least one of the amino acids; or is encoded by a nucleotide sequence set forth as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 90% identity with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

24. The corn-based flour according to claim 23, at least partially comprising a nixtamalised corn flour.

25-28. (canceled)

29. The corn-based flour according to claim 23 or claim 24 wherein a further enzyme is present in addition to the mixture of corn-based flour and xylanase enzyme.

30. The corn-based flour according to claim 23 or claim 24, further including a hydrocolloid.

31-33. (canceled)

34. The corn-based flour according to claim 30 in which the hydrocolloid is carboxymethylcellulose.

35-45. (canceled)

46. A process for the preparation of a masa foodstuff, the process comprising the steps of
(i) cooking corn in an alkaline solution;
(ii) contacting a xylanase enzyme with the corn during or after cooking,
such that a xylan-containing material native to the corn is degraded;
wherein the xylanase enzyme is selected from the group consisting of:
(B) a polypeptide as set forth in SEQ ID No. 17 or SEQ ID No. 18 or SEQ ID No. 19; or a variant, fragment, homologue or derivative thereof having at least 90% identity with SEQ ID No. 17 or SEQ ID No. 18 or SEQ ID No. 19; or encoded by a nucleotide sequence set forth as SEQ ID No. 20, SEQ ID No. 21 or SEQ ID No. 22, or is encoded by a nucleotide sequence which can hybridize to the complement of SEQ ID No. 20, SEQ ID No. 21 or SEQ ID No. 22 under high stringency conditions, or is encoded by a nucleotide sequence which has at least 90% identity with SEQ ID No. 20, SEQ ID No. 21 or SEQ ID No. 22, or is encoded by a nucleotide sequence which differs from SEQ ID No. 20, SEQ ID No. 21 or SEQ ID No. 22 due to the degeneracy of the genetic code; or
(A1) a polypeptide sequence set forth as SEQ ID No. 1, SEQ ID No. 2 or SEQ ID No. 3, or a variant, homologue, fragment or derivative thereof having at least 90% identity with SEQ ID No. 1, SEQ ID No. 2 or SEQ ID No. 3, or a polypeptide sequence which comprises SEQ ID No. 1, SEQ ID No. 2 or SEQ ID No. 3, with a conservative substitution of at least one of the amino acids, or is encoded by a nucleotide sequence set forth as SEQ ID No. 4, SEQ ID No. 5 or SEQ ID No. 6, or is encoded by a nucleotide sequence which can hybridize to SEQ ID No. 4, SEQ ID No. 5 or SEQ ID No. 6 under high stringency conditions, or is encoded by a nucleotide sequence which has at least at least 90% identity with SEQ ID No. 4, SEQ ID No. 5 or SEQ ID No. 6, or is encoded by a nucleotide sequence which differs from SEQ ID No. 4 or SEQ ID No. 5 or SEQ ID No. 6 due to the degeneracy of the genetic code; or
(C) a modified GH10 xylanase enzyme or a fragment thereof having xylanase activity wherein said modified GH10 xylanase or fragment thereof has increased thermostability compared with a parent GH10 xylanase enzyme, the parent GH10 xylanase having been modified at two or more of the following positions 7, 33, 79, 217 and 298, wherein the numbering is based on the amino acid numbering of FveXyn4 (SEQ ID No. 3) or
(A2) a polypeptide sequence set forth as SEQ ID No. 7, SEQ ID No. 8 or SEQ ID No. 9, or a variant, homologue, fragment or derivative thereof having at least 90% identity with SEQ ID No. 7, SEQ ID No. 8 or SEQ ID No. 9, or a polypeptide sequence which comprises SEQ ID No. 7, SEQ ID No. 8 or SEQ ID No. 9 with a conservative substitution of at least one of the amino acids; or is encoded by a nucleotide sequence set forth as SEQ ID No. 11, SEQ ID No. 12, SEQ ID No. 13, SEQ ID No. 14, SEQ ID No. 15 or SEQ ID No. 16, or is encoded by a nucleotide sequence which can hybridize to SEQ ID No. 11, SEQ ID No. 12, SEQ ID No. 13, SEQ ID No. 14, SEQ ID No. 15 or SEQ ID No. 16 under high stringency conditions, or is encoded by a nucleotide sequence which has at least 90% identity with SEQ ID No. 11, SEQ ID No. 12, SEQ ID No. 13, SEQ ID No. 14, SEQ ID No. 15 or SEQ ID No. 16, or is encoded by a nucleotide sequence which differs from SEQ ID No. 11, SEQ ID No. 12, SEQ ID No. 13, SEQ ID No. 14, SEQ ID No. 15 or SEQ ID No. 16 due to the degeneracy of the genetic code.

47-54. (canceled)


SEQUENCE LISTING (44 sequences)

SEQ ID NO: 1 - protein, 328 aa, Fusarium verticillioides
MKLSSFLYTASLVAAIPTAIEPRQAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

SEQ ID NO: 2 - protein, 313 aa, Fusarium verticillioides
IPTAIEPRQAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

SEQ ID NO: 3 - protein, 305 aa, Fusarium verticillioides
QAADSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKPAYTAVVNALR

SEQ ID NO: 4 - DNA, 1039 bp, Fusarium verticillioides
atgaagctgtcttctttcctctacaccgcctcgctggtcgcggccattcccaccgccatcgagccccgccaggctgccgacagcatcaacaagctgatcaagaacaagggcaagctctactacggaaccatcaccgaccccaacctgctcggcgtcgcaaaggacaccgccatcatcaaggccgactttggcgccgttacccccgagaactcgggcaagtgggacgccaccgagcccagccagggcaagttcaacttcggtagcttcgaccaggttgtcaactttgcccagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtgccgctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttacactgctgttgtcaacgctctccgctaa

SEQ ID NO: 5 - DNA, 987 bp, Fusarium verticillioides
atgaagctgtcttctttcctctacaccgcctcgctggtcgcggccattcccaccgccatcgagccccgccaggctgccgacagcatcaacaagctgatcaagaacaagggcaagctctactacggaaccatcaccgaccccaacctgctcggcgtcgcaaaggacaccgccatcatcaaggccgactttggcgccgttacccccgagaactcgggcaagtgggacgccaccgagcccagccagggcaagttcaacttcggtagcttcgaccaggttgtcaactttgcccagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtgccgctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttacactgctgttgtcaacgctctccgctaa

SEQ ID NO: 6 - DNA, 942 bp, Fusarium verticillioides
attcccaccgccatcgagccccgccaggctgccgacagcatcaacaagctgatcaagaacaagggcaagctctactacggaaccatcaccgaccccaacctgctcggcgtcgcaaaggacaccgccatcatcaaggccgactttggcgccgttacccccgagaactcgggcaagtgggacgccaccgagcccagccagggcaagttcaacttcggtagcttcgaccaggttgtcaactttgcccagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtgccgctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttacactgctgttgtcaacgctctccgctaa

SEQ ID NO: 7 - protein, 328 aa, Fusarium oxysporum
MKLSSFLYTASLVAAIPTAIEPRQASDSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTNVVGRYKGKIYAWDVVNEIFDWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKAAYTAVVNALR

SEQ ID NO: 8 - protein, 313 aa, Fusarium oxysporum
IPTAIEPRQASDSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTNVVGRYKGKIYAWDVVNEIFDWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKAAYTAVVNALR

SEQ ID NO: 9 - protein, 305 aa, Fusarium oxysporum
QASDSINKLIKNKGKLYYGTITDPNLLGVAKDTAIIKADFGAVTPENSGKWDATEPSQGKFNFGSFDQVVNFAQQNGLKVRGHTLVWHSQLPQWVKNINDKATLTKVIENHVTNVVGRYKGKIYAWDVVNEIFDWDGTLRKDSHFNNVFGNDDYVGIAFRAARKADPNAKLYINDYSLDSGSASKVTKGMVPSVKKWLSQGVPVDGIGSQTHLDPGAAGQIQGALTALANSGVKEVAITELDIRTAPANDYATVTKACLNVPKCIGITVWGVSDKNSWRKEHDSLLFDANYNPKAAYTAVVNALR

SEQ ID NO: 10 - skipped

SEQ ID NO: 11 - DNA, 1039 bp, Fusarium oxysporum
atgaagctgtcttccttcctctacaccgcctcgctggtcgcggccattcccaccgccatcgagccccgccaggcctccgacagcatcaacaagctgatcaagaacaagggcaagctctactacggaaccatcaccgaccccaacctgctcggcgtcgcaaaggacactgccatcatcaaggctgactttggcgccgtcacacccgagaactcgggtaagtgggatgccaccgagcccagccagggcaagttcaacttcggcagcttcgaccaggtcgtcaactttgctcagcagaatggcctcaaggtccgaggtcacactctagtctggcactcccagctccctcagtgggttaagaacatcaacgacaaggctactttgaccaaggtcatcgagaaccacgtcaccaacgtcgttggacgctacaagggcaagatctacgcctgggtatgttttcttcactcgaacttcttataaatggctttactaacatgttcaggacgtcgttaacgagatcttcgactgggatggtaccctccgaaaggactctcacttcaacaacgtcttcggcaacgacgactacgttggcattgccttccgcgctgcccgcaaggctgaccccaacgccaagctgtacatcaacgactacagcctcgactccggcagcgcctccaaggtcaccaagggcatggttccctctgtcaagaagtggctcagccagggcgtccccgtcgacggtattggttctcagactcaccttgaccccggtgccgctggccaaatccagggtgctctcactgccctcgccaactctggtgtgaaggaggttgccatcaccgagctcgacatccgcactgcccccgccaacgactacgctaccgttaccaaggcctgcctcaacgtccccaagtgcattggtatcaccgtctggggcgtatctgacaagaactcttggcgcaaggagcacgacagccttctgttcgatgctaactacaaccccaaggctgcttacactgctgttgtcaacgctctccgctaa

SEQ ID NO: 12 - DNA, 987 bp, Fusarium oxysporum
atgaagctgtcttccttcctctacaccgcctcgctggtcgcggccattcccaccgccatcgagccccgccaggcctccgacagcatcaacaagctgatcaagaacaagggcaagctctactacggaaccatcaccgaccccaacctgctcggcgtcgcaaaggacactgccatcatcaaggctgactttggcgccgtcacacccgagaactcgggtaagtgggatgccaccgagcccagccagggcaagttcaacttcggcagcttcgaccaggtcgtcaactttgctcagcagaatggcctcaaggtccgaggtcacactctagtctggcactcccagctccctcagtgggttaagaacatcaacgacaaggctactttgaccaaggtcatcgagaaccacgtcaccaacgtcgttggacgctacaagggcaagatctacgcctgggacgtcgttaacgagatcttcgactgggatggtaccctccgaaaggactctcacttcaacaacgtcttcggcaacgacgactacgttggcattgccttccgcgctgcccgcaaggctgaccccaacgccaagctgtacatcaacgactacagcctcgactccggcagcgcctccaaggtcaccaagggcatggttccctctgtcaagaagtggctcagccagggcgtccccgtcgacggtattggttctcagactcaccttgaccccggtgccgctggccaaatccagggtgctctcactgccctcgccaactctggtgtgaaggaggttgccatcaccgagctcgacatccgcactgcccccgccaacgactacgctaccgttaccaaggcctgcctcaacgtccccaagtgcattggtatcaccgtctggggcgtatctgacaagaactcttggcgcaaggagcacgacagccttctgttcgatgctaactacaaccccaaggctgcttacactgctgttgtcaacgctctccgctaa

SEQ ID NO: 13 - DNA, 942 bp, Fusarium oxysporum
attcccaccgccatcgagccccgccaggcctccgacagcatcaacaagctgatcaagaacaagggcaagctctactacggaaccatcaccgaccccaacctgctcggcgtcgcaaaggacactgccatcatcaaggctgactttggcgccgtcacacccgagaactcgggtaagtgggatgccaccgagcccagccagggcaagttcaacttcggcagcttcgaccaggtcgtcaactttgctcagcagaatggcctcaaggtccgaggtcacactctagtctggcactcccagctccctcagtgggttaagaacatcaacgacaaggctactttgaccaaggtcatcgagaaccacgtcaccaacgtcgttggacgctacaagggcaagatctacgcctgggacgtcgttaacgagatcttcgactgggatggtaccctccgaaaggactctcacttcaacaacgtcttcggcaacgacgactacgttggcattgccttccgcgctgcccgcaaggctgaccccaacgccaagctgtacatcaacgactacagcctcgactccggcagcgcctccaaggtcaccaagggcatggttccctctgtcaagaagtggctcagccagggcgtccccgtcgacggtattggttctcagactcaccttgaccccggtgccgctggccaaatccagggtgctctcactgccctcgccaactctggtgtgaaggaggttgccatcaccgagctcgacatccgcactgcccccgccaacgactacgctaccgttaccaaggcctgcctcaacgtccccaagtgcattggtatcaccgtctggggcgtatctgacaagaactcttggcgcaaggagcacgacagccttctgttcgatgctaactacaaccccaaggctgcttacactgctgttgtcaacgctctccgctaa

SEQ ID NO: 14 - DNA, 1039 bp, Fusarium sp.
atgaagctgtcttctttcctctacaccgcctcgctggtcgcggccattcccaccgccatcgagccccgccaggccgccgacagcatcaacaagctgatcaagaacaagggcaagctctactacggaaccatcaccgaccccaacctgctcggcgtcgcaaaggacaccgccgtcatcaaggccgactttggcgccgtcacccccgagaactcgggcaagtgggacgccaccgagcccagccagggcaacttcaacttcggtagcttcgaccaggtcgtcaactttgctcagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggtatgttttcttgcctcgaccttctcaaagatgaatttgctaacatgttcaggacgttgtcaacgagatcttcgactgggacggtaccctccgaaaggattctcacttcaacaacgtcttcggcaacgatgactacgttggcattgccttccgcgccgcccgcaaggctgaccccaacgccaagctgtacatcaacgactacagcctcgactccgccagcgcctccaaggtcaccaagggcatggtcccctccgtcaagaagtggctcagccagggcgttcccgtcgacggcattggctcccagtctcaccttgaccccggtgccgctggccaagtccagggtgctctcactgccctcgccaactctggtgtcaaggaggttgccatcaccgagctcgacatccgcactgcccccgccaacgactacgccaccgtcaccaaggcctgcctaaacgtccccaagtgcattggtatcaccgtctggggtgtctctgacaagaactcttggcgcaaggagcacgacagccttctgttcgactccaactacaaccccaagcctgcttacactgctgttgtcaacgctctccgctaa

SEQ ID NO: 15 - DNA, 987 bp, Fusarium sp.
atgaagctgtcttctttcctctacaccgcctcgctggtcgcggccattcccaccgccatcgagccccgccaggccgccgacagcatcaacaagctgatcaagaacaagggcaagctctactacggaaccatcaccgaccccaacctgctcggcgtcgcaaaggacaccgccgtcatcaaggccgactttggcgccgtcacccccgagaactcgggcaagtgggacgccaccgagcccagccagggcaacttcaacttcggtagcttcgaccaggtcgtcaactttgctcagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggacgttgtcaacgagatcttcgactgggacggtaccctccgaaaggattctcacttcaacaacgtcttcggcaacgatgactacgttggcattgccttccgcgccgcccgcaaggctgaccccaacgccaagctgtacatcaacgactacagcctcgactccgccagcgcctccaaggtcaccaagggcatggtcccctccgtcaagaagtggctcagccagggcgttcccgtcgacggcattggctcccagtctcaccttgaccccggtgccgctggccaagtccagggtgctctcactgccctcgccaactctggtgtcaaggaggttgccatcaccgagctcgacatccgcactgcccccgccaacgactacgccaccgtcaccaaggcctgcctaaacgtccccaagtgcattggtatcaccgtctggggtgtctctgacaagaactcttggcgcaaggagcacgacagccttctgttcgactccaactacaaccccaagcctgcttacactgctgttgtcaacgctctccgctaa

SEQ ID NO: 16 - DNA, 942 bp, Fusarium sp.
attcccaccgccatcgagccccgccaggccgccgacagcatcaacaagctgatcaagaacaagggcaagctctactacggaaccatcaccgaccccaacctgctcggcgtcgcaaaggacaccgccgtcatcaaggccgactttggcgccgtcacccccgagaactcgggcaagtgggacgccaccgagcccagccagggcaacttcaacttcggtagcttcgaccaggtcgtcaactttgctcagcagaatggcctcaaggtccgaggtcacactctggtctggcactctcagctccctcagtgggttaagaacatcaacgacaaggctactctgaccaaggtcattgagaaccacgtcacccaagtcgttggacgctacaagggcaagatctacgcctgggacgttgtcaacgagatcttcgactgggacggtaccctccgaaaggattctcacttcaacaacgtcttcggcaacgatgactacgttggcattgccttccgcgccgcccgcaaggctgaccccaacgccaagctgtacatcaacgactacagcctcgactccgccagcgcctccaaggtcaccaagggcatggtcccctccgtcaagaagtggctcagccagggcgttcccgtcgacggcattggctcccagtctcaccttgaccccggtgccgctggccaagtccagggtgctctcactgccctcgccaactctggtgtcaaggaggttgccatcaccgagctcgacatccgcactgcccccgccaacgactacgccaccgtcaccaaggcctgcctaaacgtccccaagtgcattggtatcaccgtctggggtgtctctgacaagaactcttggcgcaaggagcacgacagccttctgttcgactccaactacaaccccaagcctgcttacactgctgttgtcaacgctctccgctaa

SEQ ID NO: 17 - protein, 229 aa, Aspergillus clavatus
MVSFKYLFLAASALGALAAPVEVEESSWFNETALHEFAERAGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

SEQ ID NO: 18 - protein, 211 aa, Aspergillus clavatus
APVEVEESSWFNETALHEFAERAGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

SEQ ID NO: 19 - protein, 189 aa, Aspergillus clavatus
AGTPSSTGWNNGYYYSFWTDNGGTVNYQNGNGGSYSVQWKDTGNFVGGKGWNPGSARTINYSGSFNPSGNAYLTVYGWTTNPLVEYYIVENYGTYNPGNGGTYRGSVYSDGANYNIYTATRYNAPSIEGDKTFTQYWSVRQSKRTGGTVTTANHFNAWAQLGMSLGTHNYQIVATEGYQSSGSSSITVY

SEQ ID NO: 20 - DNA, 750 bp, Aspergillus clavatus
atggtgtcgttcaagtatcttttcctggcagcctctgccctgggagcgctggctgctcctgttgaagtggaagagtcttcctggttcaacgagacggctctgcacgagtttgctgagcgtgcgggcacccccagctcgaccggatggaataatggctactactattccttctggactgacaatggtggcactgttaactaccagaacggcaacggcggttcgtactcagtccagtggaaggacacaggcaactttgttggcggcaagggctggaaccctggaagcgctcggtatgtctttgtgtatctagagaaagccagagggtgtacaaagaattaaccggcatgcagaaccatcaactactcgggcagcttcaaccccagcggcaatgcctatctcaccgtgtatggctggaccactaatccccttgtggagtactacatagtggagaactatggtacttataaccctggaaatggtggcacctataggggcagtgtatactctgatggtgccaactataacatctacacggccactcgctacaatgctccctccattgagggtgataagaccttcacccagtactggtctgtgcgccagagtaagcggactggcggcacagttacaactgccaaccacttcaacgcctgggctcagctgggtatgagtttgggcacccacaactatcagatcgttgctactgagggctaccagagcagcggttcctcctccattactgtctattaa

SEQ ID NO: 21 - DNA, 690 bp, Aspergillus clavatus
atggtgtcgttcaagtatcttttcctggcagcctctgccctgggagcgctggctgctcctgttgaagtggaagagtcttcctggttcaacgagacggctctgcacgagtttgctgagcgtgcgggcacccccagctcgaccggatggaataatggctactactattccttctggactgacaatggtggcactgttaactaccagaacggcaacggcggttcgtactcagtccagtggaaggacacaggcaactttgttggcggcaagggctggaaccctggaagcgctcgaaccatcaactactcgggcagcttcaaccccagcggcaatgcctatctcaccgtgtatggctggaccactaatccccttgtggagtactacatagtggagaactatggtacttataaccctggaaatggtggcacctataggggcagtgtatactctgatggtgccaactataacatctacacggccactcgctacaatgctccctccattgagggtgataagaccttcacccagtactggtctgtgcgccagagtaagcggactggcggcacagttacaactgccaaccacttcaacgcctgggctcagctgggtatgagtttgggcacccacaactatcagatcgttgctactgagggctaccagagcagcggttcctcctccattactgtctattaa

SEQ ID NO: 22 - DNA, 636 bp, Aspergillus clavatus
gctcctgttgaagtggaagagtcttcctggttcaacgagacggctctgcacgagtttgctgagcgtgcgggcacccccagctcgaccggatggaataatggctactactattccttctggactgacaatggtggcactgttaactaccagaacggcaacggcggttcgtactcagtccagtggaaggacacaggcaactttgttggcggcaagggctggaaccctggaagcgctcgaaccatcaactactcgggcagcttcaaccccagcggcaatgcctatctcaccgtgtatggctggaccactaatccccttgtggagtactacatagtggagaactatggtacttataaccctggaaatggtggcacctataggggcagtgtatactctgatggtgccaactataacatctacacggccactcgctacaatgctccctccattgagggtgataagaccttcacccagtactggtctgtgcgccagagtaagcggactggcggcacagttacaactgccaaccacttcaacgcctgggctcagctgggtatgagtttgggcacccacaactatcagatcgttgctactgagggctaccagagcagcggttcctcctccattactgtctattaa

SEQ ID NO: 23 - DNA, 27 bp, Artificial Sequence
  Primer 1
caccatgaagctgtcttctttcctcta

SEQ ID NO: 24 - DNA, 27 bp, Artificial Sequence
  Primer 2
tttttagcggagagcgttgacaacagc

SEQ ID NO: 25 - DNA, 40 bp, Artificial Sequence
  Primer 1
ccgcggccgcaccatgaagctgtcttccttcctctacacc

SEQ ID NO: 26 - DNA, 37 bp, Artificial Sequence
  Primer 2
ccggcgcgcccttattagcggagagcgttgacaacag

SEQ ID NO: 27 - DNA, 39 bp, Artificial Sequence
  Primer 1
ccgcggccgcaccatggtgtcgttcaagtatcttttcct

SEQ ID NO: 28 - DNA, 40 bp, Artificial Sequence
  Primer 2
ccggcgcgcccttattaatagacagtaatggaggaggaac

SEQ ID NO: 29 - DNA, 987 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagccccagcagggcaagttcaccttcggtagcttcgaccaggttgtcaactttgcccagcagaatggcctctacgtccgaggtcacactctggtctggcacggccagctccctcagtgggttaagaacatcaacgacaaggctatgctgaccaaggtcattgagaaccacgtcacccaactcgttggacgctacaagggcaagatctacgcctgggacgtcgtcaacgagatcttcgagtgggacggtaccctccgaaaggactctcacttcaaccaggtcttcggcaacgacgactacgttggcattgccttccgcgccgcccgcaaggctgaccccaacgccaagctgtacatcaacgactacagcctcgactcccagagcgcctccaaggtcaccaagggtatggttccctacgtcaagaagtggctcagccagggcgttcccgtcgacggcattggctctcagactcaccttgaccccggtcaggctccccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 30 - DNA, 987 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagccccagcagggcaagttcaccttcaccagcttcgaccaggttgtcaactttgcccagcagaatggcctctacgtccgaggtcacactctggtctggcactctcagctccctcagtgggttaagaacatcaacgacaaggctatgctgaccaaggtcattgagaaccacgtcacccaactcgttggacgctacaagggcaagatctacgcctgggacgtcgtcaacgagatcttcgagtgggacggtaccctccgaaaggactctcacttcaaccaggtcttcggcaacgacgactacgttggcattgccttccgcgccgcccgcaaggctgaccccaacgccaagctgtacatcaacgactacagcctcgactcccagagcgcctccaaggtcaccaagggtatggttccctacgtcaagaagtggctcagccagggcgttcccgtcgacggcattggctctcagactcaccttgaccccggtcaggctccccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 31 - DNA, 987 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccctctgctcggcgtcgcaaaggacgtcgccatcatcaaggccgactttggcgccgttacccccgagaactcgggcaagtgggacgccaccgagcccagccagggcaagttcaacttcggtagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 32 - DNA, 987 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgaccccaacctgctcggcgtcgcaaaggacgtcgccatcatcaaggccgactttggcgccgttacccccgagaactcgggcaagtgggacgccaccgagccccagcagggcaagttcaccttcaccagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 33 - DNA, 987 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagcccagccagggcaagttcaacttcaccagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 34 - DNA, 1039 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagccccagcagggcaagttcaccttcggtagcttcgaccaggttgtcaactttgcccagcagaatggcctctacgtccgaggtcacactctggtctggcacggccagctccctcagtgggttaagaacatcaacgacaaggctatgctgaccaaggtcattgagaaccacgtcacccaactcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaaccaggtcttcggcaacgacgactacgttggcattgccttccgcgccgcccgcaaggctgaccccaacgccaagctgtacatcaacgactacagcctcgactcccagagcgcctccaaggtcaccaagggtatggttccctacgtcaagaagtggctcagccagggcgttcccgtcgacggcattggctctcagactcaccttgaccccggtcaggctccccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 35 - DNA, 1039 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagccccagcagggcaagttcaccttcaccagcttcgaccaggttgtcaactttgcccagcagaatggcctctacgtccgaggtcacactctggtctggcactctcagctccctcagtgggttaagaacatcaacgacaaggctatgctgaccaaggtcattgagaaccacgtcacccaactcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaaccaggtcttcggcaacgacgactacgttggcattgccttccgcgccgcccgcaaggctgaccccaacgccaagctgtacatcaacgactacagcctcgactcccagagcgcctccaaggtcaccaagggtatggttccctacgtcaagaagtggctcagccagggcgttcccgtcgacggcattggctctcagactcaccttgaccccggtcaggctccccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 36 - DNA, 1039 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccctctgctcggcgtcgcaaaggacgtcgccatcatcaaggccgactttggcgccgttacccccgagaactcgggcaagtgggacgccaccgagcccagccagggcaagttcaacttcggtagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 37 - DNA, 1039 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgaccccaacctgctcggcgtcgcaaaggacgtcgccatcatcaaggccgactttggcgccgttacccccgagaactcgggcaagtgggacgccaccgagccccagcagggcaagttcaccttcaccagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 38 - DNA, 1039 bp, Artificial Sequence
  Variant GH10 xylanase sequence
atgaagctgtcttctttcctctacaccgcctcgctggtcgcggccattcccaccgccatcgagccccgccaggctgccgacagcatcgacaagctgatcaagaacaagggcaagctctactacggaaccatcaccgacccccccctgctcggcgtcgcaaaggacgtcgccatcatcaaggccgactttggcgccgttacccccgagaactcgggcaagtgggacgccaccgagcccagccagggcaagttcaacttcaccagcttcgaccaggttgtcaactttgcccagcagaatggcctctacgtccgaggtcacactctggtctggcacggccagctccctcagtgggttaagaacatcaacgacaaggctactctgaccaaggtcattgagaaccacgtcacccaagtcgttggacgctacaagggcaagatctacgcctgggtatgttttattcccccagacttcttcgaaatgactttgctaacatgttcaggacgtcgtcaacgagatcttcgagtgggacggtaccctccgaaaggactctcacttcaacaacgtcttcggcaacgacgactacgttggcattgccttccgcgccgcccgcaaggctgaccccaacgccaagctgtacatcaacgactacagcctcgactccggcagcgcctccaaggtcaccaagggtatggttccctccgtcaagaagtggctcagccagggcgttcccgtcgacggcattggctctcagactcaccttgaccccggtcaggctggccaaatccagggtgctctcactgccctcgccaattctggtgtcaaggaggttgccatcaccgagctcgacatccgcactgcccccgccaacgactacgctaccgtcaccaaggcctgcctcaacgtccccaagtgcattggtatcaccgtctggggtgtctctgacaagaactcttggcgcaaggagcacgacagtcttctgttcgatgctaactacaaccccaagcctgcttactacgctgttgtcaacgctctccgctaa

SEQ ID NO: 39 - protein, 305 aa, Artificial Sequence
  Variant GH10 xylanase sequence
QAADSIDKLIKNKGKLYYGTITDPPLLGVAKDVAIIKADFGAVTPENSGKWDATEPQQGKFTFGSFDQVVNFAQQNGLYVRGHTLVWHGQLPQWVKNINDKAMLTKVIENHVTQLVGRYKGKIYAWDVVNEIFEWDGTLRKDSHFNQVFGNDDYVGIAFRAARKADPNAKLYINDYSLDSQSASKVTKGMVPYVKKWLSQGVPVDGIGSQTHLDPGQAPQIQGALTALANSGVKEVAITELDIRTAPANDYATVTKACLNVPKCIGITVWGVSDKNSWRKEHDSLLFDANYNPKPAYYAVVNALR

SEQ ID NO: 40 - protein, 305 aa, Artificial Sequence
  Variant GH10 xylanase sequence
QAADSIDKLIKNKGKLYYGTITDPPLLGVAKDVAIIKADFGAVTPENSGKWDATEPQQGKFTFTSFDQVVNFAQQNGLYVRGHTLVWHSQLPQWVKNINDKAMLTKVIENHVTQLVGRYKGKIYAWDVVNEIFEWDGTLRKDSHFNQVFGNDDYVGIAFRAARKADPNAKLYINDYSLDSQSASKVTKGMVPYVKKWLSQGVPVDGIGSQTHLDPGQAPQIQGALTALANSGVKEVAITELDIRTAPANDYATVTKACLNVPKCIGITVWGVSDKNSWRKEHDSLLFDANYNPKPAYYAVVNALR

SEQ ID NO: 41 - protein, 305 aa, Artificial Sequence
  Variant GH10 xylanase sequence
QAADSIDKLIKNKGKLYYGTITDPPLLGVAKDVAIIKADFGAVTPENSGKWDATEPSQGKFNFGSFDQVVNFAQQNGLYVRGHTLVWHGQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGQAGQIQGALTALANSGVKEVAITELDIRTAPANDYATVTKACLNVPKCIGITVWGVSDKNSWRKEHDSLLFDANYNPKPAYYAVVNALR

SEQ ID NO: 42 - protein, 305 aa, Artificial Sequence
  Variant GH10 xylanase sequence
QAADSIDKLIKNKGKLYYGTITDPNLLGVAKDVAIIKADFGAVTPENSGKWDATEPQQGKFTFTSFDQVVNFAQQNGLYVRGHTLVWHGQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGQAGQIQGALTALANSGVKEVAITELDIRTAPANDYATVTKACLNVPKCIGITVWGVSDKNSWRKEHDSLLFDANYNPKPAYYAVVNALR

SEQ ID NO: 43 - protein, 305 aa, Artificial Sequence
  Variant GH10 xylanase sequence
QAADSIDKLIKNKGKLYYGTITDPPLLGVAKDVAIIKADFGAVTPENSGKWDATEPSQGKFNFTSFDQVVNFAQQNGLYVRGHTLVWHGQLPQWVKNINDKATLTKVIENHVTQVVGRYKGKIYAWDVVNEIFEWDGTLRKDSHFNNVFGNDDYVGIAFRAARKADPNAKLYINDYSLDSGSASKVTKGMVPSVKKWLSQGVPVDGIGSQTHLDPGQAGQIQGALTALANSGVKEVAITELDIRTAPANDYATVTKACLNVPKCIGITVWGVSDKNSWRKEHDSLLFDANYNPKPAYYAVVNALR

SEQ ID NO: 44 - protein, 305 aa, Fusarium sp.
QAADSINKLIKNKGKLYYGTITDPNLLGVAKDTAVIKADFGAVTPENSGKWDATEPSQGNFNFGSFDQVVNFAQQNGLKVRGHTLVWHSQLPQWVKNINDKATLTKVIENHVTQVVGRYKGKIYAWDVVNEIFDWDGTLRKDSHFNNVFGNDDYVGIAFRAARKADPNAKLYINDYSLDSASASKVTKGMVPSVKKWLSQGVPVDGIGSQSHLDPGAAGQVQGALTALANSGVKEVAITELDIRTAPANDYATVTKACLNVPKCIGITVWGVSDKNSWRKEHDSLLFDSNYNPKPAYTAVVNALR